(19) United States
(12) Patent Application Publication
(10) Pub. No.: US 2022/0347596 A1
(43) Pub. Date: Nov. 3, 2022

(54) INDUSTRIAL-SCALE PROCESSING OF CANNABIS MATERIAL

(71) Applicant: HEXO OPERATIONS INC., Ottawa (CA)

(72) Inventors: Max Alsayar, Ottawa (CA); George Elvira, Montreal (CA); Stephan Georgiev, Saint-Hubert (CA); David Walters, Stittsville (CA)

(21) Appl. No.: 17/621,194

(22) PCT Filed: Jun. 19, 2020
(86) PCT No.: PCT/CA2020/050861

§ 371 Date: Dec. 20, 2021

Related U.S. Application Data
(60) Provisional application No. 62/864,594, filed on Jun. 21, 2019.

(51) Int. Cl. B01D 11/02; A23L 33/105

(52) U.S. Cl. CPC B01D 11/0257; B01D 11/0288; B01D 11/0203; B01D 11/0284; A23L 33/105

(57) ABSTRACT

The present application relates to processing of cannabis material, particularly on a large scale, such as at an industrial level. Cannabis is typically a controlled and regulated substance, and has traditionally been processed in low quantities. A human-based manual and/or labour-intensive processing implementation is not scalable, and is therefore infeasible at an industrial level. Disclosed herein are systems and methods for facilitating industrial-scale processing of cannabis material.

### CROSS-REFERENCE TO RELATED APPLICATION

This application is related to, and claims priority to, United States Provisional Patent Application No. 62/864,594, entitled “INDUSTRIAL-SCALE PROCESSING OF CANNABIS MATERIAL”, and filed on Jun. 21, 2019, the entire contents of which are incorporated by reference herein.

### FIELD

This disclosure relates generally to processing of cannabis material. In particular, the disclosure relates to systems and methods for processing cannabis material on an industrial scale, in continuous and/or integrated processes or systems in some embodiments.

### BACKGROUND

Cannabis materials, such as cannabis plant material and other materials that are derived from cannabis plant material, are typically processed in a segmented and non-continuous “batch” process in which the cannabis materials are moved from one finite processing station to another in batches. As each station completes its processing of a batch of input material, personnel move a batch of processed material to a next station. This requires allocating personnel not only to operate each station, but also to transfer materials from one station to the next.

Also, due to stringent regulatory requirements such as those in respect of weight and loss traceability, a batch process incurs greater cost for operation. It is estimated that 50-60% of employee time is spent on administrative documentation of tracking cannabis material.

### SUMMARY

Conventional batch processing tends to be slow and inefficient in that each station completes its processing of a batch of input material before any processed material is provided to a next station so that the next station can begin its processing. This can introduce time delay and/or inefficiencies in usage of processing stations if, for example, a processing station is idle during a time when it has no input material to process because a previous processing station from which it receives input material has not yet completed processing of a batch of cannabis material.

Human intervention required in conventional batch processing is also prone to increased operation cost, contamination risk, and human error as a result of the high level of involvement of personnel in such processing.

Some embodiments disclosed herein propose establishing fluid communication and/or other transfer means or mechanisms between cannabis material processing stations so that materials flow through a processing system without the need for human intervention to physically move those materials during processing.

Some embodiments propose also or instead integrating cannabis material processing stations that are separate stations in conventional processing systems.

At least some aspects of processing are automated in some embodiments. For example, electronic equipment and/or components that execute software are used in some embodiments for such purposes as coordinating or synchronizing processing by stations and/or operation of transfer mechanisms such as fluid pumps to streamline and potentially optimize processing.

Regarding employee time spent on administrative documentation for tracking cannabis material as noted above, it is expected that a continuous and/or otherwise integrated, automated, or streamlined process or system would significantly reduce the amount of this administrative work, by half or more.

One particular aspect of the present disclosure relates to a system comprising: a first station to reduce size of a cannabis plant material; and a second station, coupled to receive reduced size cannabis plant material from the first station, to obtain from the reduced size cannabis plant material a cannabis extract including at least one cannabinoid and/or terpene. The second station may be coupled to receive a continuous supply of reduced size cannabis plant material, for example.

In some embodiments, the second station is in fluid communication with the first station.

In some embodiments, the second station is coupled to the first station via a transfer mechanism configured for transferring the reduced size cannabis plant material from the first station to the second station.

In some embodiments, the transfer mechanism comprises a vessel to hold the reduced size cannabis plant material from the first station before transfer to the second station.

In some embodiments, the transfer mechanism comprises a conveyor.

In some embodiments, the transfer mechanism comprises a pipe.

In some embodiments, the second station is configured to obtain the cannabis extract by performing mechanical extraction on the reduced size cannabis plant material.

In some embodiments, the second station is configured to obtain the cannabis extract by extracting the reduced size cannabis plant material with an extraction solvent.

In some embodiments, the second station is configured for contacting the reduced size cannabis plant material with the extraction solvent.

In some embodiments, the first station is configured for contacting the cannabis plant material with the extraction solvent.

In some embodiments, the extraction solvent transfers the reduced size cannabis plant material from the first station to the second station.

In some embodiments, the extracting comprises a warm solvent extraction process that further causes decarboxylation of the at least one cannabinoid.

In some embodiments, the system further comprises a winterization station, coupled to receive the cannabis extract from the second station, to winterize the cannabis extract. The winterization station may be coupled to receive a continuous supply of the cannabis extract from the second station.

In some embodiments, the winterization station is in fluid communication with the second station.

In some embodiments, the winterization station is coupled to the second station via a transfer mechanism configured for transferring the cannabis extract from the second station to the winterization station.

In some embodiments, the transfer mechanism configured for transferring the cannabis extract from the second station to the winterization station comprises a pipe.

In some embodiments, the transfer mechanism configured for transferring the cannabis extract from the second station to the winterization station comprises a vessel to hold the cannabis extract from the second station before transfer to the winterization station.

In some embodiments, the system further comprises: a winterization station, coupled to receive the cannabis extract from the second station, to winterize the cannabis extract, and the extraction solvent transfers the cannabis extract from the second station to the winterization station. The winterization station may be coupled to receive a continuous supply of the cannabis extract from the second station.

In some embodiments, the winterization station is configured for contacting the cannabis extract with a winterization solvent.

In some embodiments, the system further comprises a distillation station, coupled to receive winterized cannabis extract from the winterization station, to purify the at least one cannabinoid and/or terpene. The distillation station may be coupled to receive a continuous supply of the winterized cannabis extract from the winterization station.

In some embodiments, the distillation station is in fluid communication with the winterization station.

In some embodiments, the distillation station is coupled to the winterization station via a transfer mechanism configured for transferring the winterized cannabis extract from the winterization station to the distillation station.

In some embodiments, the transfer mechanism configured for transferring the winterized cannabis extract from the winterization station to the distillation station comprises a pipe.

In some embodiments, the transfer mechanism is configured for transferring the winterized cannabis extract from the winterization station to the distillation station comprises a vessel to hold the winterized cannabis extract from the winterization station before transfer to the distillation station.

In some embodiments, the system further comprises a distillation station, coupled to receive winterized cannabis extract from the winterization station, to purify the at least one cannabinoid and/or terpene, and the winterization solvent transfers the winterized cannabis extract from the winterization station to the distillation station. The distillation station may be coupled to receive a continuous supply of the winterized cannabis extract from the winterization station.

In some embodiments, the system further comprises a distillation station, coupled to receive the cannabis extract from the second station, to purify the at least one cannabinoid and/or terpene. The distillation station may be coupled to receive a continuous supply of the cannabis extract from the second station.

In some embodiments, the distillation station is in fluid communication with the second station.

In some embodiments, the distillation station is coupled to the second station via a transfer mechanism configured for transferring the cannabis extract from the second station to the distillation station.

In some embodiments, the transfer mechanism is configured for transferring the cannabis extract from the second station to the distillation station comprises a pipe.

In some embodiments, the transfer mechanism configured for transferring the cannabis extract from the second station to the distillation station comprises a vessel to hold the cannabis extract from the second station before transfer to the distillation station.

In some embodiments, the system further comprises a distillation station, coupled to receive the cannabis extract from the second station, to purify the at least one cannabinoid and/or terpene, and the extraction solvent transfers the cannabis extract from the second station to the distillation station. The distillation station may be coupled to receive a continuous supply of the cannabis extract from the second station.

In some embodiments, the system further comprises a separation station, coupled to receive the cannabis extract from the second station, to separate the at least one cannabinoid and/or terpene from the cannabis extract.

In some embodiments, the system further comprises a separation station, coupled to receive winterized cannabis extract from the winterization station, to separate the at least one cannabinoid and/or terpene from the winterized cannabis extract.

In some embodiments, the system further comprises a separation station, coupled to receive a distillate from the distillation station, to further purify the at least one cannabinoid and/or terpene.

In some embodiments, the system further comprises a pre-treatment station to pre-treat the cannabis plant material, and the first station is coupled to receive pre-treated cannabis plant material from the pre-treatment station and reduce size of the pre-treated cannabis plant material.

Another aspect of the present disclosure relates to a method comprising: processing a cannabis plant material at a first station, to reduce size of the cannabis plant material and produce reduced size cannabis plant material; and processing the reduced size cannabis plant material, at a second station that is coupled to receive the reduced size cannabis plant material from the first station, to obtain from the reduced size cannabis plant material a cannabis extract including at least one cannabinoid and/or terpene. The second station may be coupled to receive a continuous supply of reduced size cannabis plant material, for example.

In some embodiments, the second station is in fluid communication with the first station.

In some embodiments, the method further comprises controlling a transfer mechanism to transfer the reduced size cannabis plant material from the first station to the second station.

In some embodiments, processing the reduced size cannabis plant material at the second station comprises performing mechanical extraction on the reduced size cannabis plant material.

In some embodiments, processing the reduced size cannabis plant material at the second station comprises extracting the reduced size cannabis plant material with an extraction solvent.

In some embodiments, the extracting comprises contacting the reduced size cannabis plant material with the extraction solvent.

In some embodiments, processing the cannabis plant material at the first station comprises contacting the cannabis plant material with the extraction solvent to transfer the reduced size cannabis plant material from the first station to the second station.

In some embodiments, the extracting comprises a warm solvent extraction process that further causes decarboxylation of the at least one cannabinoid.

In some embodiments, the method further comprises processing the cannabis extract, at a winterization station that is coupled to receive the cannabis extract from the second station, to winterize the cannabis extract. The winterization station may be coupled to receive a continuous supply of the cannabis extract, for example.

In some embodiments, the winterization station is in fluid communication with the second station.

In some embodiments, the method further comprises controlling a transfer mechanism to transfer the cannabis extract from the second station to the winterization station.

In some embodiments, the method further comprises: transferring the cannabis extract, in a continuous supply for example, from the second station to a winterization station using the extraction solvent; and processing the cannabis extract, at the winterization station, to winterize the cannabis extract.

In some embodiments, processing the cannabis extract at the winterization station comprises contacting the cannabis extract with a winterization solvent.

In some embodiments, the method further comprises processing winterized cannabis extract, at a distillation station that is coupled to receive winterized cannabis extract from the winterization station, to purify the at least one cannabinoid and/or terpene. The distillation station may be coupled to receive a continuous supply of the winterized cannabis extract from the winterization station, for example.

In some embodiments, the distillation station is in fluid communication with the winterization station.

In some embodiments, the method further comprises controlling a transfer mechanism to transfer the winterized cannabis extract from the winterization station to the distillation station.

In some embodiments, the method further comprises: transferring winterized cannabis extract, in a continuous supply for example, from the winterization station to a distillation station using the winterization solvent; and processing the winterized cannabis extract, at the distillation station, to purify the at least one cannabinoid and/or terpene.

In some embodiments, the method further comprises processing the cannabis extract, at a distillation station that is coupled to receive the cannabis extract from the second station, to purify the at least one cannabinoid and/or terpene. The distillation station may be coupled to receive a continuous supply of the cannabis extract from the second station, for example.

In some embodiments, the distillation station is in fluid communication with the second station.

In some embodiments, the method further comprises controlling a transfer mechanism to transfer the cannabis extract from the second station to the distillation station.

In some embodiments, the method further comprises: transferring the cannabis extract, in a continuous supply for example, from the second station to a distillation station using the extraction solvent; and processing the cannabis extract, at the distillation station, to purify the at least one cannabinoid and/or terpene.

In some embodiments, the method further comprises processing the cannabis extract, at a separation station that is coupled to receive the cannabis extract from the second station, to separate the at least one cannabinoid and/or terpene from the cannabis extract.

In some embodiments, the method further comprises processing winterized cannabis extract, at a separation station that is coupled to receive the winterized cannabis extract from the winterization station, to separate the at least one cannabinoid and/or terpene from the winterized cannabis extract.

In some embodiments, the method further comprises processing a distillate, at a separation station that is coupled to receive the distillate from the distillation station, to further purify the at least one cannabinoid and/or terpene.

In some embodiments, the method further comprises pre-treating the cannabis plant material at pre-treatment station, and the processing at the first station comprises processing pre-treated cannabis plant material from the pre-treatment station.

A system according to a further aspect of the present disclosure includes one or more controllers to control operation of a first station to reduce size of a cannabis plant material, and to control operation of a second station that is coupled to receive a continuous supply of reduced size cannabis plant material from the first station and to obtain from the reduced size cannabis plant material a cannabis extract including at least one cannabinoid and/or terpene.

The one or more controllers may be configured to coordinate operation of the first station and operation of the second station with the continuous supply.

The one or more controllers may include a controller to coordinate, with operation of the first station and operation of the second station, operation of a transfer mechanism to transfer the reduced size cannabis plant material from the first station to the second station.

The one or more controllers may include a controller to coordinate operation of one or more further stations with each other and/or with operation of either or both of the first station and the second station.

In an embodiment, the one or more controllers include a controller to coordinate operation of one or more transfer mechanisms to transfer cannabis material to or from the one or more further stations with operation of the one or more further stations and/or with operation of either or both of the first station and the second station.

The one or more further stations may include any one or more of: a decarboxylation station; a winterization station; a distillation station; a separation station; and a pre-treatment station, for example.

A method according to yet another aspect of the present disclosure involves controlling processing of a cannabis plant material at a first station to reduce size of the cannabis plant material and produce reduced size cannabis plant material; and controlling processing of the reduced size cannabis plant material at a second station that is coupled to receive a continuous supply of the reduced size cannabis plant material from the first station and to obtain from the reduced size cannabis plant material a cannabis extract including at least one cannabinoid and/or terpene.

Controlling processing at the first station and controlling processing at the second station may involve coordinating the processing at the first station and the processing at the second station with the continuous supply.

Such a method may involve controlling transfer of the reduced size cannabis plant material from the first station to the second station.

In some embodiments, a method involves coordinating processing at one or more further stations with each other and/or with the processing at either or both of the first station and the second station.

Some embodiments may involve coordinating transfer of cannabis material to or from the one or more further stations with the processing at the one or more further stations and/or with the processing at either or both of the first station and the second station. As noted above, the one or more further stations may include any one or more of: a decarboxylation station; a winterization station; a distillation station; a separation station; and a pre-treatment station, for example.

Another aspect of the present disclosure relates to a system comprising: a first station to process a cannabis plant material to obtain a cannabis extract including at least one cannabinoid and/or terpene; and a second station, coupled to receive the cannabis extract from the first station, to purify the cannabis extract. The cannabis extract is continuously transferred from the first station in some embodiments.

The system may also include a transfer mechanism, coupled to the first station and to the second station, to continuously transfer at least a portion of the cannabis extract from the first station to the second station.

The first station may be configured to obtain the cannabis extract by processing the cannabis plant material with an extraction solvent, and the transfer mechanism may be configured to transfer at least the portion of the cannabis extract to the second station in at least a portion of the extraction solvent.

The first station may be configured to obtain the cannabis extract by performing mechanical extraction on the cannabis plant material.

In some embodiments, the first station comprises: a first substation to reduce size of the cannabis plant material; and a second substation, coupled to receive reduced size cannabis plant material from the first substation, to obtain the cannabis extract from the reduced size cannabis plant material.

In some embodiments, the first station comprises: a pre-treatment substation to pre-treat cannabis plant material; and an extraction substation, coupled to receive pre-treated cannabis plant material from the pre-treatment substation, to obtain the cannabis extract from the pre-treated cannabis plant material.

In some embodiments, the first station comprises: a pre-treatment substation to pre-treat cannabis plant material; a first substation, coupled to receive pre-treated cannabis plant material from the pre-treatment substation, to reduce size of the pre-treated cannabis plant material; and a second substation, coupled to receive reduced size cannabis plant material from the first substation, to obtain the cannabis extract from the reduced size cannabis plant material.

In some embodiments, the second station comprises a winterization substation to process the cannabis extract and obtain a winterized extract.

The winterization substation may be configured to winterize the cannabis extract in presence of a winterization solvent to obtain the winterized extract.

In some embodiments, the second station comprises a distillation substation to process the cannabis extract and obtain the at least one cannabinoid and/or terpene.

In some embodiments, the second station further comprises a distillation substation, coupled to receive the winterized extract from the winterization substation, to process the winterized extract and obtain the at least one cannabinoid and/or terpene.

A system may include a transfer mechanism, coupled to the winterization substation and to the distillation substation, to transfer the winterized extract to the distillation substation.

In some embodiments, the second station comprises a separation substation to process the cannabis extract and obtain the at least one cannabinoid and/or terpene.

In some embodiments, the second station further comprises a separation substation, coupled to receive the winterized extract from the winterization substation, to process the winterized extract and obtain the at least one cannabinoid and/or terpene.

A system may include a transfer mechanism, coupled to the winterization substation and to the separation substation, to transfer the winterized extract to the separation substation.

In some embodiments, the second station further comprises a separation substation, coupled to receive from the distillation substation a distillate comprising the at least one cannabinoid and/or terpene, to process the distillate and further purify the at least one cannabinoid and/or terpene.

A system may include a transfer mechanism, coupled to the separation substation and to the distillation substation, to transfer the distillate to the separation substation.

In some embodiments, the first station includes an extraction vessel to hold the cannabis extract in an extraction solvent; and a transfer mechanism coupled to the extraction vessel and configured to continuously withdraw a portion of the extraction solvent containing the cannabis extract from the extraction vessel so as to substantially maintain at least a minimum volume of plant material and extraction solvent in the extraction vessel. The transfer mechanism may be configured to continuously withdraw the portion of the extraction solvent containing the cannabis extract from the extraction vessel so as to substantially maintain a constant volume of plant material and extraction solvent in the extraction vessel.

The second station may include a winterization substation coupled to the transfer mechanism, to receive the withdrawn portion of the extraction solvent containing the cannabis extract.

The winterization substation may be configured to contact the extract with a winterization solvent.

In some embodiments, a distillation substation is coupled to the transfer mechanism, to receive the withdrawn portion of the extraction solvent containing the cannabis extract. The second station may include a separation substation in fluid communication with the distillation substation. A transfer mechanism may be coupled to the separation substation and to the distillation substation, to transfer a distillate from the distillation substation to the separation substation.

A separation substation may be coupled to the transfer mechanism, to receive the withdrawn portion of the extraction solvent containing the cannabis extract.

The second station may include a distillation substation in fluid communication with the winterization substation. A transfer mechanism may be coupled to the winterization substation and to the distillation substation, to transfer winterized extract to the distillation station.

In some embodiments, a separation substation is in fluid communication with the winterization substation. A transfer mechanism may be coupled to the winterization substation and to the separation station, to transfer winterized extract to the separation station.

Another aspect of the present disclosure relates to a method comprising: processing a cannabis plant material at a first station to obtain a cannabis extract including at least one cannabinoid and/or terpene; and processing the cannabis extract, at a second station that is coupled to receive the cannabis extract from the first station, to purify the cannabis extract. The second station may be coupled to receive the cannabis extract that is continuously transferred from the first station, for example.

A method may include continuously transferring at least a portion of the cannabis extract from the first station to the second station.

The processing at the first station may involve processing the cannabis plant material with an extraction solvent, and continuously transferring may involve transferring at least the portion of the cannabis extract to the second station in at least a portion of the extraction solvent.

In an embodiment, the processing at the first station involves performing mechanical extraction on the cannabis plant material.

In some embodiments, the processing at the first station comprises: processing the cannabis plant material at a first substation of the first station to reduce size of the cannabis plant material; and processing reduced size cannabis plant material from the first substation, at a second substation of the first station that is coupled to receive the reduced size cannabis plant material from the first substation, to obtain the cannabis extract from the reduced size cannabis plant material.

In some embodiments, the processing at the first station comprises: pre-treating cannabis plant material at a pre-treatment substation; and processing pre-treated cannabis plant material from the pre-treatment substation, at an extraction substation of the first station that is coupled to receive the pre-treated cannabis plant material from the pre-treatment substation, to obtain the cannabis extract from the pre-treated cannabis plant material.

In some embodiments, the processing at the first station comprises: pre-treating cannabis plant material at a pre-treatment substation; processing pre-treated cannabis plant material at a first substation that is coupled to receive the pre-treated cannabis plant material from the pre-treatment substation, to reduce size of the pre-treated cannabis plant material; and processing reduced size cannabis plant material from the first substation, at a second substation of the first station that is coupled to receive the reduced size cannabis plant material from the first substation, to obtain the cannabis extract from the reduced size cannabis plant material.

In some embodiments, the processing at the second station comprises winterizing the cannabis extract to obtain a winterized extract. The winterizing may involve winterizing the cannabis extract in presence of a winterization solvent to obtain the winterized extract.

In some embodiments, processing at the second station comprises distilling the cannabis extract to obtain the at least one cannabinoid and/or terpene.

In some embodiments, processing at the second station further comprises distilling the winterized extract to obtain the at least one cannabinoid and/or terpene.

In some embodiments, the processing at the second station comprises performing separation to separate the at least one cannabinoid and/or terpene in the cannabis extract and obtain the at least one cannabinoid and/or terpene.

In some embodiments, the processing at the second station further comprises performing separation to separate the at least one cannabinoid and/or terpene in the winterized extract and obtain the at least one cannabinoid and/or terpene.

In some embodiments, the processing at the second station further comprises performing separation to further purify the at least one cannabinoid and/or terpene by separating the at least one cannabinoid and/or terpene in a distillate comprising the at least one cannabinoid and/or terpene.

The first station may include an extraction vessel to hold the cannabis extract in an extraction solvent, and a method may involve continuously withdrawing a portion of the extraction solvent containing the cannabis extract from the extraction vessel so as to substantially maintain at least a minimum volume of plant material and extraction solvent in the extraction vessel. Continuously withdrawing may involve continuously withdrawing the portion of the extraction solvent containing the cannabis extract from the extraction vessel so as to substantially maintain a constant volume of plant material and extraction solvent in the extraction vessel.

The second station may include a winterization substation in fluid communication with the extraction vessel, and a method may involve transferring the extract from the extraction vessel to the winterization substation.

The withdrawn portion of the extraction solvent may transfer the extract from the extraction vessel to the winterization substation.

A method may involve incorporating a winterization solvent such that the extract is in contact with the winterization solvent in the winterization substation.

In some embodiments, a method involves winterizing the extract.

The second station further comprises a distillation substation in fluid communication with the winterization substation. A method may involve transferring winterized extract from the winterization substation to the distillation substation. In some embodiments, a method involves distillation of the winterized extract to purify the at least one cannabinoid and/or terpene.

In an embodiment, the second station includes a distillation substation in fluid communication with the extraction vessel. The withdrawn portion of the extraction solvent may transfer the extract from the extraction vessel to the distillation substation. In some embodiments, a method involves distillation of the extract to purify the at least one cannabinoid and/or terpene.

A method may involve separation of the at least one cannabinoid and/or terpene in the cannabis plant extract to obtain the at least one cannabinoid and/or terpene.

In an embodiment, a method involves separation of the at least one cannabinoid and/or terpene in winterized extract from the winterization substation.

A method may involve separation of a distillate comprising the at least one cannabinoid and/or terpene, to further purify the at least one cannabinoid and/or terpene.

Another aspect of the present disclosure relates to a method comprising: processing a cannabis plant material at an extraction station to obtain a cannabis extract including at least one cannabinoid and/or terpene; and continuously transferring at least a portion of the cannabis extract to a purification station that is coupled to receive the cannabis extract from the extraction station.

In some embodiments, the processing at an extraction station comprises processing the cannabis plant material with an extraction solvent, and wherein the transferring comprises transferring at least the portion of the cannabis extract in at least a portion of the extraction solvent.

In some embodiments, the processing at an extraction station comprises performing mechanical extraction on the cannabis plant material.

In some embodiments, the purification station comprises a winterization station, and the method further comprises winterizing the cannabis extract in presence of a winterization solvent to obtain a winterized extract.

In some embodiments, the purification station further comprises a distillation station, and the method further comprises distillation of the winterized extract to obtain the at least one cannabinoid and/or terpene.

In some embodiments, the purification station comprises a distillation station, and the method further comprises distillation of the cannabis extract to obtain the at least one cannabinoid and/or terpene.

In some embodiments, the purification station comprises a separation station, and the method further comprises separation of the at least one cannabinoid and/or terpene in the cannabis extract to obtain the at least one cannabinoid and/or terpene from the cannabis extract.

In some embodiments, the purification station further comprises a separation station, and the method further comprises separation of the at least one cannabinoid and/or terpene in the winterized cannabis extract to obtain the at least one cannabinoid and/or terpene from the winterized cannabis extract.

In some embodiments, the purification station further comprises a separation station, and the method further comprises separation of the at least one cannabinoid and/or terpene in a distillate, to further purify the at least one cannabinoid and/or terpene.

Another aspect of the present disclosure relates to a system comprising: an extraction station to obtain from a cannabis plant material a cannabis extract including at least one cannabinoid and/or terpene; a purification station to purify the cannabis extract; and a transfer mechanism, coupled to the extraction station and to the purification station, to continuously transfer at least a portion of the cannabis extract from the extraction station to the purification station.

In some embodiments, the extraction station is configured to obtain the cannabis extract by processing the cannabis plant material with an extraction solvent, and the transfer mechanism is configured to transfer at least the portion of the cannabis extract to the purification station in at least a portion of the extraction solvent.

In some embodiments, the extraction station is configured to obtain the cannabis extract by performing mechanical extraction on the cannabis plant material.

In some embodiments, the purification station comprises a winterization station to winterize the cannabis extract in presence of a winterization solvent to obtain a winterized extract.

In some embodiments, the purification station further comprises a distillation station, coupled to receive the winterized extract from the winterization station, to distill the winterized extract to obtain the at least one cannabinoid and/or terpene.

In some embodiments, the purification station comprises a distillation station, coupled to receive the cannabis extract from the extraction station, to distill the cannabis extract to obtain the at least one cannabinoid and/or terpene.

In some embodiments, the purification station comprises a separation station, coupled to receive the cannabis extract from the extraction station, to separate the at least one cannabinoid and/or terpene in the cannabis extract and obtain the at least one cannabinoid and/or terpene from the cannabis extract.

In some embodiments, the purification station further comprises a separation station, coupled to receive the winterized extract from the winterization station, to separate the at least one cannabinoid and/or terpene in the winterized cannabis extract and obtain the at least one cannabinoid and/or terpene from the winterized cannabis extract.

In some embodiments, the purification station further comprises a separation station, coupled to receive from the distillation station a distillate comprising the at least one cannabinoid and/or terpene, to further purify the at least one cannabinoid and/or terpene by separating the at least one cannabinoid and/or terpene in the distillate.

Another aspect of the present disclosure relates to a process comprising: providing an extraction vessel containing a cannabis plant extract in an extraction solvent; incorporating a cannabis plant material and a volume of extraction solvent into the vessel; and continuously withdrawing a portion of the extraction solvent containing the cannabis plant extract from the vessel so as to substantially maintain a constant volume of plant material and extraction solvent in the vessel, wherein the cannabis plant extract includes at least one cannabinoid and/or terpene.

An embodiment may involve providing an extraction vessel to hold a cannabis plant extract in an extraction solvent; and continuously withdrawing a portion of the extraction solvent containing the cannabis plant extract from the vessel so as to substantially maintain at least a minimum volume of plant material and extraction solvent in the extraction vessel, wherein the cannabis plant extract includes at least one cannabinoid and/or terpene. Continuously withdrawing may involve continuously withdrawing the portion of the extraction solvent containing the cannabis plant extract from the vessel so as to substantially maintain a constant volume of the plant material and extraction solvent in the extraction vessel.

In some embodiments, the extraction vessel is in fluid communication with a winterization station.

In some embodiments, the process further comprises transferring the extract from the extraction vessel to the winterization station.

In some embodiments, the withdrawn portion of the extraction solvent transfers the extract from the extraction vessel to the winterization station.

In some embodiments, the process further comprises incorporating a winterization solvent such that the extract is in contact with the winterization solvent in the winterization station.

In some embodiments, the process further comprises winterizing the extract to obtain a winterized extract.

In some embodiments, the winterization station is in fluid communication with a distillation station.

In some embodiments, the process further comprises transferring winterized extract from the winterization station to the distillation station.

In some embodiments, the process further comprises distillation of the winterized extract to purify the at least one cannabinoid and/or terpene.

In some embodiments, the extraction vessel is in fluid communication with a distillation station.

In some embodiments, the withdrawn portion of the extraction solvent transfers the extract from the extraction vessel to the distillation station.

In some embodiments, the process further comprises distillation of the extract to purify the at least one cannabinoid and/or terpene.

In some embodiments, the process further comprises separation of the at least one cannabinoid and/or terpene in the cannabis plant extract to obtain the at least one cannabinoid and/or terpene.

In some embodiments, the process further comprises separation of the at least one cannabinoid and/or terpene in winterized extract from the winterization station, for example to purify the at least one cannabinoid and/or terpene.

In some embodiments, the process further comprises separation of a distillate comprising the at least one cannabinoid and/or terpene, to further purify the at least one cannabinoid and/or terpene.

Another aspect of the present disclosure relates to a system comprising: an extraction vessel containing a cannabis plant extract in an extraction solvent; and a transfer mechanism coupled to the extraction vessel and configured to continuously withdraw a portion of the extraction solvent containing the cannabis plant extract from the vessel so as to substantially maintain a constant volume of plant material and extraction solvent in the vessel, wherein the cannabis plant extract includes at least one cannabinoid and/or terpene.

In an embodiment, a system includes an extraction vessel to hold a cannabis plant extract in an extraction solvent; and a transfer mechanism coupled to the extraction vessel and configured to continuously withdraw a portion of the extraction solvent containing the cannabis plant extract from the vessel so as to substantially maintain at least a minimum volume of plant material and extraction solvent in the extraction vessel, wherein the cannabis plant extract includes at least one cannabinoid and/or terpene. The transfer mechanism may be configured to continuously withdraw the portion of the extraction solvent containing the cannabis plant extract from the extraction vessel so as to substantially maintain a constant volume of plant material and extraction solvent in the extraction vessel.

In some embodiments, the system further comprises a winterization station coupled to the transfer mechanism, to receive the withdrawn portion of the extraction solvent containing the cannabis plant extract.

In some embodiments, the winterization station is configured to contact the extract with a winterization solvent.

In some embodiments, the system further comprises a distillation station in fluid communication with the winterization station.

In some embodiments, the system further comprises a transfer mechanism, coupled to the winterization station and to the distillation station, to transfer the winterized extract to the distillation station.

In some embodiments, the system further comprises a distillation station coupled to the transfer mechanism, to receive the withdrawn portion of the extraction solvent containing the cannabis plant extract.

In some embodiments, the system further comprises a separation station coupled to the transfer mechanism, to receive the withdrawn portion of the extraction solvent containing the cannabis plant extract.

In some embodiments, the system further comprises a separation station in fluid communication with the winterization station.

A transfer mechanism may be coupled to the winterization station and to the separation station, to transfer winterized extract to the separation station.

In some embodiments, the system further comprises a separation station in fluid communication with the distillation station.

A transfer mechanism may be coupled to the separation station and to the distillation station, to transfer a distillate from the distillation station to the separation station.

A system according to a further aspect of the present disclosure includes one or more controllers to control operation of a first station to process cannabis plant material to obtain a cannabis extract including at least one cannabinoid and/or terpene, and to control operation of a second station that is coupled to receive a continuous transfer of the cannabis extract from the first station and to purify the cannabis extract.

The one or more controllers may be configured to coordinate operation of the first station and operation of the second station with continuous transfer of the cannabis extract.

The first station may include an extraction vessel to hold the cannabis extract in an extraction solvent, and the one or more controllers may include a controller to control continuous withdrawal of a portion of the extraction solvent containing the cannabis extract from the extraction vessel so as to substantially maintain at least a minimum volume of plant material and extraction solvent in the extraction vessel.

A method according to yet another aspect of the present disclosure involves: controlling operation of a first station to process cannabis plant material to obtain a cannabis extract including at least one cannabinoid and/or terpene; and controlling operation of a second station that is coupled to receive the cannabis extract continuously transferred from the first station and to purify the cannabis extract.

Controlling operation of the first station and controlling operation of the second station may involve coordinating operation of the first station and operation of the second station with continuous transfer of the cannabis extract.

The first station may include an extraction vessel to hold the cannabis extract in an extraction solvent, and such a method may involve controlling continuous withdrawal of a portion of the extraction solvent containing the cannabis extract from the extraction vessel so as to substantially maintain at least a minimum volume of plant material and extraction solvent in the extraction vessel.

Another aspect of the present disclosure relates to a process for removing an undesirable component from a cannabis plant extract, the cannabis plant extract including an extraction solvent, with one or more cannabinoids and the undesirable component in solution in the extraction solvent, the undesirable component having a precipitation temperature at which the one or more cannabinoids remain in solution in the extraction solvent, the process comprising: continuously supplying cannabis plant extract to a precipitation separator that comprises a cooling path to cool the cannabis plant extract, as the cannabis plant extract is passing through the cooling path at a flow rate, to induce precipitation of the undesirable component; controlling a rate of heat extraction from the cooling path in relation to the flow rate to bring the cannabis plant extract passing through the cooling path to a temperature that is below the precipitation temperature; and removing precipitated undesirable component from cooled cannabis plant extract.

The precipitation separator may be or be part of a winterization station. The process may involve controlling a rate of transfer of the cannabis plant extract to the precipitation separator to substantially match a rate of winterization.

A process may involve controlling the flow rate.

Controlling the flow rate may involve controlling the flow rate using one or more valves at one or both of an inlet of the cooling path and an outlet of the cooling path. Controlling the flow rate may also or instead involve controlling the flow rate using one or more pumps.

The cannabis plant extract is gravity fed through the cooling path in some embodiments.

A process may involve adjusting any one or more of: an angle of the cooling path with respect to vertical, shape of the cooling path, size of the cooling path, and drag exerted on the cannabis plant extract by the cooling path, to control the flow rate.

The adjusting may involve adjusting the drag exerted on the cannabis plant extract by the cooling path by changing a width or a cross-sectional area of the cooling path, for example.

In some embodiments, heat is extracted from the cooling path using a heat exchanger.

Removing the undesirable component may involve repeatedly or continuously removing the undesirable component from the cooled cannabis extract as it flows through the cooling path.

The removing may involve filtering.

In an embodiment, the removing involves using one or more filters.

The removing may also or instead involve using one or more membranes.

The removing may involve a brush or filter periodically or continuously sweeping to catch or trap the undesirable component.

A process may involve depositing the undesirable component in a container.

A system for removing an undesirable component from a cannabis plant extract is also disclosed. The cannabis plant extract includes an extraction solvent, with one or more cannabinoids and the undesirable component in solution in the extraction solvent. The undesirable component has a precipitation temperature at which the one or more cannabinoids remain in solution in the extraction solvent. In an embodiment, the system includes: a precipitation separator to receive a continuous supply of cannabis plant extract, the precipitation separator comprising a cooling path to cool the cannabis plant extract, as the cannabis plant extract passes through the cooling path at a flow rate, to induce precipitation of the undesirable component; and a controller to control a rate of heat extraction from the cooling path in relation to the flow rate to bring the cannabis plant extract passing through the cooling path to a temperature that is below the precipitation temperature.

The precipitation separator may be or be part of a winterization station.

The controller or a further controller may be configured to control a rate of transfer of the cannabis plant extract to the precipitation separator to substantially match a rate of winterization.

The controller or a further controller may be configured to control the flow rate. The controller or the further controller may be configured to control the flow rate using valves at one or both of an inlet of the cooling path and an outlet of the cooling path. The controller or the further controller may be configured to control the flow rate by also or instead controlling the flow rate using one or more pumps.

The cannabis plant extract may be gravity fed through the cooling path in such a system.

The controller or a further controller may be configured to control the flow rate by adjusting any one or more of: angle of the cooling path with respect to vertical, shape of the cooling path, size of the cooling path, and drag exerted on the cannabis plant extract by the cooling path, to control the flow rate. In an embodiment, the controller or the further controller is configured to control the flow rate by adjusting the drag exerted on the cannabis plant extract by the cooling path by changing a width or a cross-sectional area of the cooling path.

A system may include a heat exchanger to extract heat from the cooling path.

In an embodiment, a system includes an element for removal of precipitated undesirable component from cooled cannabis plant extract as it flows through the cooling path.

The element may include one or more filters.

The element may also or instead include one or more membranes.

The element may also or instead include one or more centrifuges.

The element may include a brush.

A system may include a container, and a pipe to enable the undesirable component to be removed and to deposit the undesirable component in the container.

In an embodiment, a system includes an output coupled to an input of a heating element to allow winterized cannabis plant extract to enter the heating element.

Such a system may include a filter to prevent the undesirable component from flowing into the heating element.

The winterized cannabis plant extract flows to the heating element in a continuous stream in some embodiments.

A method according to another aspect of the present disclosure involves: controlling continuous supply of cannabis plant extract to a precipitation separator that comprises a cooling path to cool the cannabis plant extract, as the cannabis plant extract passes through the cooling path at a flow rate, to induce precipitation of an undesirable component from the cannabis plant extract, the cannabis plant extract including an extraction solvent, with one or more cannabinoids and the undesirable component in solution in the extraction solvent, the undesirable component having a precipitation temperature at which the one or more cannabinoids remain in solution in the extraction solvent; and controlling a rate of heat extraction from the cooling path in relation to the flow rate to bring the cannabis plant extract passing through the cooling path to a temperature that is below the precipitation temperature.

The precipitation separator may be or be part of a winterization station, and a method may involve controlling a rate of transfer of the cannabis plant extract to the precipitation separator to substantially match a rate of winterization.

A method may involve controlling the flow rate.

Controlling the flow rate may involve controlling the flow rate using one or more valves at one or both of an inlet of the cooling path and an outlet of the cooling path, and/or using one or more pumps.

A method may involve coordinating processing of cannabis material at one or more further stations with each other and/or with processing of the cannabis plant extract at a winterization station that includes the precipitation separator.

In an embodiment, a method involves coordinating transfer of cannabis material to or from the one or more further stations with the processing at the one or more further stations and/or with the processing of the cannabis plant extract at the winterization station.

The one or more further stations may include, for example, any one or more of: a pre-treatment station; a milling station; an extraction station; a decarboxylation station; a distillation station; and a separation station.

A system may include one or more controllers to: control continuous supply of cannabis plant extract to a precipitation separator that comprises a cooling path to cool the cannabis plant extract, as the cannabis plant extract passes through the cooling path at a flow rate, to induce precipitation of an undesirable component from the cannabis plant extract, the cannabis plant extract including an extraction solvent, with one or more cannabinoids and the undesirable component in solution in the extraction solvent, the undesirable component having a precipitation temperature at which the one or more cannabinoids remain in solution in the extraction solvent; and to control a rate of heat extraction from the cooling path in relation to the flow rate to bring the cannabis plant extract passing through the cooling path to a temperature that is below the precipitation temperature.

The precipitation separator may be or be part of a winterization station, and the one or more controllers may include a controller to control a rate of transfer of the cannabis plant extract to the precipitation separator to substantially match a rate of winterization.

The one or more controllers may include a controller to control the flow rate.

The controller to control the flow rate may be configured to control the flow rate using one or more valves at one or both of an inlet of the cooling path and an outlet of the cooling path, and/or using one or more pumps.

The one or more controllers may include a controller to coordinate processing of cannabis material at one or more further stations with each other and/or with processing of the cannabis plant extract at a winterization station that includes the precipitation separator.

The one or more controllers may include a controller to coordinate transfer of cannabis material to or from the one or more further stations with the processing at the one or more further stations and/or with the processing of the cannabis plant extract at the winterization station.

The one or more further stations may include any one or more of: a pre-treatment station; a milling station; an extraction station; a decarboxylation station; a distillation station; and a separation station, for example.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

For a more complete understanding of the present disclosure, reference is now made to the following description taken in conjunction with the accompanying drawings, in which:

FIG. 1 is a flow diagram illustrating an example process for producing cannabis products by processing cannabis material in accordance with an embodiment;

FIG. 2 is a block diagram illustrating an example system for producing cannabis products in accordance with an embodiment;

FIG. 3 is a block diagram illustrating an integrated system for production of cannabis products according to another embodiment;

FIGS. 4A-4E are block diagrams illustrating an example automated cannabis material processing system;

FIG. 5 is a flow diagram illustrating a method according to another embodiment;

FIG. 6 is a flow diagram illustrating a method according to a further embodiment.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

For illustrative purposes, specific example embodiments will be explained in greater detail below in conjunction with the figures. It should be appreciated, however, that the present disclosure provides many applicable concepts that can be embodied in any of a wide variety of specific contexts. The specific embodiments discussed are merely illustrative and do not limit the scope of the present disclosure. For example, embodiments could include additional, different, or fewer features than shown in the drawings.

The present disclosure relates, in part, to the production of one or more cannabis products by processing one or more cannabis materials. The term “cannabis product(s)” includes goods that are produced from cannabis or hemp, which include plant material, oils, resins, drinks, food additives, edibles, creams, aerosol sprays and vaporization substances, for example. The term “cannabis material(s)” includes cannabis plant material, which refers to plants or parts thereof, and/or materials that are derived from cannabis plant material and are intended for further processing to produce one or more cannabis products.

A cannabis material or product could include a cannabinoid in its pure or isolated form, or a source material that includes a cannabinoid. Examples of source materials include cannabis or hemp plant material (for example, flowers, seeds, trichomes, and kief), milled cannabis or hemp plant material, extracts obtained from cannabis or hemp plant material (for example, resins, waxes and concentrates), and distilled extracts. In some embodiments, pure or isolated cannabinoids and/or source materials comprising cannabinoids could be combined with water, lipids, hydrocarbons (for example, butane), ethanol, acetone, isopropanol, or mixtures thereof.

The term “cannabis plant” encompasses wild type cannabis sativa, cannabis indica, cannabis afghanica, and other variants thereof, including cannabis species or chemovars which naturally contain different amounts of individual cannabinoids. For example, some cannabis strains have been bred to produce minimal levels of THC, the principal psychoactive constituent responsible for the high associated with cannabis, and other strains have been selectively bred to produce high levels of THC and other psychoactive cannabinoids. Also included are hemp plants and cannabis subspecies and plants which are the result of genetic crosses, self-crosses or hybrids thereof. The term “cannabis extract” is also to be interpreted accordingly as encompassing material extracted from one or more cannabis plants.

A particular substance could be considered a cannabis product in some embodiments and a cannabis material in other embodiments. For example, a cannabis extract could be produced as a cannabis product in some embodiments, or further processed to produce a cannabis product in the form of a cannabis distillate in other embodiments.

Uses of cannabis products include medical and/or recreational uses. Large-scale production of cannabis products is expected to focus primarily, if not exclusively, on cannabis products that include active substances such as cannabinoids. However, cannabis products might not always include an active substance.

As used herein, the term “cannabinoid” is generally understood to include any chemical compound that acts upon a cannabinoid receptor. For the purpose of this specification, the expression “cannabinoid” means a compound such as tetrahydrocannabinol (THC), cannabidiol (CBD), cannabigerolic acid (CBGA), cannabigerol (CBG), cannabigerol monomethylether (CBGM), cannabigerovarin (CBGV), cannabichromene (CBC), cannabichromevarin (CBCV), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo- delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1- benzoxocin-5-methanol (OH-iso-HHCV), cannabiripsol (CBR), trihydroxy-delta-9- tetrahydrocannabinol (triOH-THC), cannabinol propyl variant (CBNV), and derivatives thereof.

In some embodiments, the cannabinoid is cannabidiol (CBD). For the purpose of this specification, the expressions “cannabidiol” or “CBD” are generally understood to refer to one or more of the following compounds, and, unless a particular other stereoisomer or stereoisomers are specified, includes the compound “Δ2- cannabidiol.” These compounds are: (1) Δ5-cannabidiol (2-(6-isopropenyl-3-methyl-5- cyclohexen-1-yl)-5-pentyl-1,3-benzenediol); (2) Δ4-cannabidiol (2-(6-isopropenyl-3- methyl-4-cyclohexen-1-yl)-5-pentyl-1,3-benzenediol); (3) Δ3-cannabidiol (2-(6- isopropenyl-3-methyl-3-cyclohexen-1-yl)-5-pentyl-1,3-benzenediol); (4) Δ3,7-cannabidiol (2-(6-isopropenyl-3-methylenecyclohex-1-yl)-5-pentyl-1,3-benzenediol); (5) Δ2- cannabidiol (2-(6-isopropenyl-3-methyl-2-cyclohexen-1-yl)-5-pentyl-1,3-benzenediol); (6) Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3- benzenediol).

In some embodiments, the cannabinoid is tetrahydrocannabinol (THC). THC is only psychoactive in its decarboxylated state. The carboxylic acid form (THCA) is non-psychoactive. Delta-9-tetrahydrocannabinol (Δ9-THC) and delta-8- tetrahydrocannabinol (Δ8-THC) produce the effects associated with cannabis by binding to the CB1 cannabinoid receptors in the brain.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

As used herein, the term “terpene” (or “decarboxylated terpene”, which is known as a terpenoid) is generally understood to include any organic compound derived biosynthetically from units of isoprene. Terpenes may be classified in various ways, such as by their sizes. For example, suitable terpenes may include monoterpenes, sesquiterpenes, or triterpenes. At least some terpenes are expected to interact with, and potentiate the activity of, cannabinoids. Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, hum ulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof.

Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270.

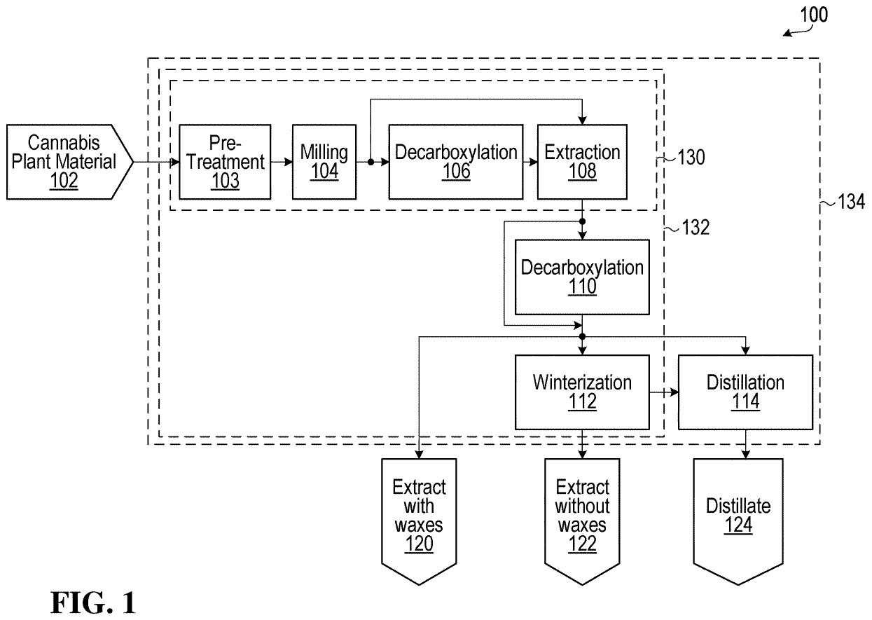

FIG. 1 is a flow diagram illustrating an example process 100 for producing cannabis products by processing cannabis material. In FIG. 1, a rectangle generally denotes a step, apparatus, device, location or operation, and a pentagon generally denotes an input material, a processed material, or a final output product. Process 100 includes one or more pre-treatment operations 103, an operation 104 of milling, operations 106, 110 of decarboxylation, an operation 108 of extraction, an operation 112 of winterization, and an operation 114 of distillation. The example process 100 shown in FIG. 1 is intended to be illustrative and non-limiting. Other embodiments include fewer, additional, and/or different operations. For example, not all embodiments necessarily involve pre-treatment at 103. Decarboxylation is performed at 106 and/or 110 in some embodiments, but in other embodiments decarboxylation is also or instead performed on an extract. Some embodiments do not involve any decarboxylation at all during processing. Winterization at 112 and distillation at 114 are also optional purification operations. Some embodiments include either or both of these operations, and other embodiments include other purification operations such as isolation or separation, in combination with or instead of winterization at 112 and/or distillation at 114. Chromatography is another example of a purification operation or process that may also or instead be used in some embodiments.

In the example process 100, cannabis plant material 102 is a source or starting material for process 100 in the example shown. In some embodiments, cannabis plant material 102 is produced and harvested in a cannabis grow area, and then transferred into process 100. Other possible sources of cannabis plant material 102 include a producer and/or a supplier of cannabis, from which cannabis plant material is received. Cannabis plant material 102 is intended to include any material that originated from a cannabis plant, including cannabis flower, trim and/or waste for example. Cannabis flower could also be referred to as bud, and is typically harvested from mature cannabis plants. Trim includes the leaves of the cannabis plant that are separated from the flower and stems. Trim could be harvested before the flower, while plants mature. Waste could include roots, stalks, stems and leaves that were not separated into trim, for example. In some embodiments, cannabis plant material 102 is received from a plant part separation process that separates harvested cannabis plants into flower, trim and/or waste.

Process 100 begins with harvesting, receiving, or otherwise providing a supply of cannabis plant material 102. Such a supply of cannabis plant material 102 is continuous in some embodiments, to feed the process 100 as plant material is needed. In other embodiments, the supply of cannabis plant material 102 is semi-continuous and provided in batches while process 100 is ongoing so that the cannabis plant material does not run out. Therefore, at least a portion of process 100 could be active and processing material when cannabis plant material 102 is received. It should be noted however, that not all embodiments involve a continuous supply of cannabis plant material 102 or continuous processing of such material. Semi-continuous or substantially continuous operation is also possible. For example, in some embodiments processing is continuous for a certain amount of time or material and one or more parts or stations in a processing line are then taken offline or shut down for cleaning, between different strains of cannabis plant material for instance.

Examples of optional pre-treatment operations at 103 include drying, freezing, or dewaxing (e.g., using chemical and/or enzymatic dewaxing). Any one or more of these pre-treatments could be applied at 103, depending on the specific cannabis product(s) to be produced, for example. Pre-treatment at 103 is tailored to specific extract products in some embodiments.

In some embodiments, drying involves air drying cannabis plant material, on one or more trays or racks for example, with or without the application of heat by a heater. Chemical treatment, in a treatment vessel or reactor vessel for example, to remove water or reduce water content is also possible. Freezing cannabis plant material, in a chiller or freezer for example, makes the cannabis plant material more brittle and can also or instead be performed at 103 to prepare the cannabis plant material for milling. Dewaxing and digestion involve enzyme and/or chemical treatment, such as in a treatment vessel or reactor vessel, to reduce wax content in the case of dewaxing for example, in some embodiments.

Cannabis plant material 102, which has been pre-processed in some embodiments, is sent for milling, also referred to as shredding, at operation 104. Operation 104 could include processing at least a portion of cannabis plant material 102 using a milling machine, for example, to reduce the physical size of the cannabis plant material and produce reduced size cannabis plant material. Reduced size cannabis plant material could increase the efficiency of other processes such as extraction, relative to process efficiency for non-milled cannabis plant material. Examples of processing of cannabis plant material 102 using a milling machine include manual processing, automated processing, and combined processing that is partially manual and partially automated.

In some embodiments, milling at 104 is performed until a predetermined size of cannabis plant material has been reached. Milling time is another possible control parameter, and milling is performed for a predetermined milling time in other embodiments.

Operation 104 is illustrative of an operation for which flow rate or feed rate control for input material is potentially beneficial. For example, instead of loading a milling machine with a batch of cannabis plant material 102, in an embodiment cannabis plant material 102 is transferred to the milling machine at a rate that is controlled to substantially match a rate of milling at the milling machine and potentially avoid bottlenecks at the milling stage 104 in process 100.

In the present disclosure, “matching” and “substantially matching” flow rates and/or processing rates refer to matching such rates to within a range that avoids or at least reduces overflow/backup/oversupply of cannabis plant material during processing and/or underflow/shortage/undersupply of cannabis plant material during processing. Such rates need not be matched exactly, and as described elsewhere herein components such as vessels are used in some embodiments to accommodate at least some rate mismatch in a processing system. It should also be appreciated that rates to be matched are not necessarily quantified using the same units. For example, in an embodiment an input rate for extraction is based on a unit of weight per a unit of time, whereas an output rate for extraction is based on a unit of volume per a unit of time, and those rates can still be matched to each other even though the flow rate units are different.

In some embodiments, the transfer of cannabis plant material 102 to the milling operation 104 is, at least in part, automated. Consider, for example, an embodiment in which cannabis plant material 102 is loaded into a hopper or other vessel, and the vessel is controlled to supply cannabis plant material to a milling machine. The vessel could be considered a reservoir or buffer to provide a continuous supply of cannabis plant material 102 for the milling operation. The vessel itself need not necessarily be fed continuously, and is at least partially filled with cannabis plant material 102 intermittently in some embodiments. In some embodiments, a vessel is periodically emptied for cleaning and then refilled with cannabis plant material. Manual and/or automated cannabis plant material supply vessel filling and/or refilling operations are possible.

In some embodiments, the vessel is coupled to a transfer mechanism, to transfer the cannabis plant material from the vessel to the milling operation. As used herein, “transfer mechanism” is intended to denote a device or apparatus that moves cannabis material from one location to another. In some embodiments, a transfer mechanism is used to help move matter from one processing operation and/or machine to a different processing operation and/or machine.

Examples of vessels and transfer mechanisms are discussed in further detail elsewhere herein.

Reduced size cannabis plant material is transferred from milling operation 104 to decarboxylation operation 106 in some embodiments, to produce reduced size decarboxylated cannabis plant material. Decarboxylation is a process in which acid forms of cannabinoids are converted to their neutral forms. More specifically, decarboxylation involves a chemical reaction that removes a carboxyl group from cannabinoids and releases CO₂.

THC and CBD are two of the main medicinally active constituents in cannabis. However, these constituents are present as the biologically inactive carboxylic acids in cannabis plants. When extracting THC or CBD from cannabis plants, it has been the practice to convert the storage precursor compounds of THCA and CBDA into their more readily extractable and pharmacologically active forms. THC and CBD acids slowly decarboxylate over time, and applying heat increases the rate of decarboxylation.

In some embodiments, decarboxylation of cannabinoid acids is a function of time and temperature. At higher temperatures a shorter period of time will be taken for complete decarboxylation of a given amount of cannabinoid acid. In selecting appropriate conditions for decarboxylation consideration must, however, be given to minimizing thermal degradation of the desirable cannabinoids into undesirable degradation products, particularly thermal degradation of THC to cannabinol (CBN). Heat need not necessarily be applied during decarboxylation.

Referring again to FIG. 1, in some embodiments process 100 involves transferring reduced size cannabis plant material from milling operation 104 to a decarboxylation device for carrying out operation 106. In some embodiments, decarboxylation involves heating the cannabis plant material, using a heater such as a decarboxylation oven or a heat tunnel. A heat tunnel or other heater in which cannabis plant material is heated as it is moved through the heater may be preferred in a continuous process. If the cannabis plant material for decarboxylation is in solution or suspension or is otherwise carried in or by a solvent, then a Continuous Stirred-Tank Reactor (CSTR) or Plug Flow Reactor (PFR), for example, could be used in performing decarboxylation operation 106. A fluidized bed reactor is another example of a device that could be used in some embodiments of decarboxylation. One or more of these types of reactors are used to implement other vessels disclosed herein, in some embodiments,

In some embodiments, at least a portion of the transfer of reduced size cannabis plant material to a decarboxylation process is automated. For example, in an embodiment a decarboxylation device is coupled to a milling machine via a transfer mechanism that is configured for transferring the reduced size cannabis plant material from the milling machine to the decarboxylation device.

Reduced size cannabis plant material need not necessarily remain in motion during the entirety of a transfer between milling operation 104 and decarboxylation operation 106. For example, in some embodiments the reduced size cannabis plant material from milling operation 104 is held in a vessel after milling but before transfer to the decarboxylation operation 106. The vessel in this example is a form of reservoir or buffer to provide a continuous supply of reduced size cannabis plant material for the decarboxylation operation 106. For example, the vessel feeds a decarboxylation device using a transfer mechanism in an embodiment. The rate of transfer of reduced size cannabis plant material to the decarboxylation operation 106 is controlled in some embodiments to substantially match a rate of decarboxylation at the decarboxylation operation 106 and/or a rate of milling operation 104, to potentially avoid bottlenecks around the decarboxylation operation in process 100.

In some embodiments, decarboxylation begins by setting a decarboxylation device to a temperature of 150° C. Cannabis plant material might not be transferred to the decarboxylation device until it has reached a minimum temperature of 120° C. A temperature probe or thermometer inserted into the cannabis plant material enables monitoring of temperature of the cannabis plant material during decarboxylation, and provides for control of heating of the cannabis plant material in the decarboxylation device until the cannabis plant material reaches a predefined temperature, such as the temperature at which the decarboxylation process occurs. In some embodiments, the predefined temperature is 120° C. For cannabis plant material and an oven or heat tunnel, an example decarboxylation temperature range is 80° C. to 150° C. For CSTR or PFR decarboxylation of cannabis plant material that is in solution or carried by a solvent, for example, decarboxylation is performed within a temperature range of 60° C. to 150° C. in some embodiments.

The temperature of the cannabis plant material is maintained within a certain range of a predefined temperature in some embodiments, such as within 4° C. of 120° C.

Heating the cannabis plant material to temperatures that exceed this range of the predefined temperature might be undesirable. Such temperatures could induce other reactions, such as vaporization of cannabinoids and terpenes, which might affect the properties of a processed material and/or a final cannabis product. In some embodiments, if the cannabis plant material reaches temperatures greater than 125° C., the set point temperature of the heater is decreased.

Weight of the cannabis plant material is also or instead monitored during decarboxylation in some embodiments, using one or more scales for example. Decarboxylation can then be controlled based on weight of the cannabis plant material, in addition to or instead of temperature.

Control of a decarboxylation device, such as control of the temperature of the decarboxylation device, is automated in some embodiments.

It should be noted that decarboxylation at operation 106 might not be performed in all embodiments. For example, reduced size cannabis material from operation 104 could bypass operation 106, as illustrated in FIG. 1, and instead proceed directly to operation 108. Moreover, some embodiments of the present disclosure relate to processes for producing cannabis materials or products that do not include an operation for decarboxylation before an operation for extraction.

The extraction at operation 108 includes processing cannabis plant material to obtain from the cannabis plant material one or more cannabis extracts that include at least one cannabinoid and/or at least one terpene. In some embodiments, the cannabis plant material processed in operation 108 includes reduced size cannabis plant material from operation 104 and/or decarboxylated cannabis plant material from operation 106. Examples of cannabis extracts include oils and non-oils such as resins.

In some embodiments, operation 108 involves a fluid extraction process, such as a solvent extraction process to obtain a cannabis extract by extracting cannabis plant material with an extraction solvent. Solvent extraction involves extracting one or more separate compounds from a source material based on solubility of each compound in an extraction solvent. A solvent extraction process includes processing or contacting cannabis plant material with an extraction solvent, which separates one or more cannabinoids and/or terpenes from the cannabis plant material and captures them in the form of a cannabis extract. Any cannabis material that remains after extraction is either treated as waste or subject to further processing.

In some embodiments, operation 108 includes solvent extraction using ethanol as the extraction solvent. In some embodiments, operation 108 includes supercritical fluid extraction using supercritical CO₂ as an extraction solvent. Other examples of extraction solvents include water, hexane, propane, pentane, butane, acetone, and other hydrocarbons. However, the embodiments described herein are not limited to any specific extraction solvents, or even to a solvent extraction. Features disclosed herein in the context of ethanol may also be applicable to other solvents, and/or to a solution of a solvent with one or more other compounds such as water. Ethanol is intended to be a representative example of a solvent, and references to a solvent or any particular solvent such as ethanol are intended to be inclusive of solvent solutions. Solvents are fluids, but could be liquid or gas.

For solvent extraction, operation 108 involves some sort of extractor or extraction vessel to contact cannabis plant material with an extraction solvent. In an embodiment, an extractor is provided to transfer extraction solvent into contact with the cannabis plant material. Examples of other features provided by an extractor in some embodiments include pressure control, temperature control, extraction fluid flow rate control and/or control of other parameters of an extraction process. In some embodiments, running an extractor is at least partially an automated process, involving an operating program for the extractor that defines parameters for an extraction run, including one or more of time duration(s), extraction solvent(s) flow rate(s), temperature(s) and pressure(s), for example. Such an operating program is stored in memory and/or on a controller of the extractor, for example, and the controller or another component that executes the operating program controls one or more components of the extractor during a run.

In fluid extraction using ethanol, ethanol immerses and/or flows through cannabis plant material, and captures cannabinoids, terpenes, and/or other substances such as waxes in the cannabis plant material that are soluble in ethanol. In some embodiments, a mixture of ethanol and cannabis plant material is agitated by an extractor to encourage dissolution of at least cannabinoids, and possibly terpenes, in the ethanol.

Fluid extraction using ethanol under warm conditions (above room temperature, >25 ° C.), room temperature conditions (20-25 ° C.), cool conditions (below room temperature, <20° C.) or super-cooled conditions (<−20° C., e.g., cooled in dry- ice) are possible. In an embodiment, ethanol is boiled in a flask or pot, cooled in a condensing coil, and then dripped through cannabis plant material to capture cannabinoids and terpenes. Such a warm ethanol process improves efficiency of fluid extraction, in terms of extraction time and/or amount of ethanol consumed for example, at least for cannabinoids and terpenes that have a higher solubility in warm ethanol. Decarboxylation is another potential benefit of warm-ethanol extraction, in that warm ethanol can cause decarboxylation of the extracted cannabinoid(s), as part of extraction rather than in a separate process. Therefore, in some embodiments, an extracting operation involves a warm solvent extraction process that further causes decarboxylation of at least one cannabinoid in a cannabis extract.

Although fluid extraction using ethanol is possible under room temperature, cool and/or super-cooled conditions, the efficiency of an extraction process in terms of extraction time and ethanol consumed, for example, is potentially reduced relative to fluid extraction under warm conditions as a result of lower solubility of cannabinoids, terpenes and/or waxes in cooler ethanol.

In supercritical fluid extraction with CO₂, an extraction run involves sealing an extraction chamber that contains cannabis plant material, and allowing the extraction chamber to fill up with CO₂, by adjusting inlet and outlet regulating valves on the extractor for example. In some embodiments, a CO₂ monitor is used to monitor the amount of CO₂ in the extraction chamber. After the extraction chamber is filled to a target CO₂ level or concentration and has reached a stable pressure, as monitored by one or more pressure sensors, a chamber heater is started. In some embodiments, the chamber is left for a predefined time, such as 30 minutes, to allow the chamber to reach a stable temperature, as monitored by one or more temperature sensors for example.

With stable temperature and pressure, an extractor could then be run to produce extract from the cannabis plant material. Running an extractor could include further adjusting heat and/or pressure in the extractor to convert gaseous CO₂ into a supercritical fluid that dissolves cannabinoids and/or terpenes in the cannabis plant material. After the extraction run is complete, the extraction chamber could be purged with CO₂ to collect the produced cannabis extract.

At an input side of operation 108, in some embodiments reduced size cannabis plant material from operation 104 is transferred to the extraction operation, and in other embodiments decarboxylated cannabis plant material from operation 106 is also or instead transferred to the extraction process. This transfer is at least partially automated in some embodiments.

For example, an extractor could be coupled to a decarboxylation heater via a transfer mechanism that is configured for transferring the decarboxylated cannabis plant material from the decarboxylation device to the extractor. An extractor could also or instead be coupled to a milling machine via another transfer mechanism configured for transferring the reduced size cannabis plant material from the milling machine to the extractor. The rate(s) of transfer could be controlled to substantially match a rate of extraction in the extractor and thereby potentially avoid processing bottlenecks and/or cannabis material supply shortages or underflows.

Processed cannabis plant material from operation 104 and/or operation 106 could be held in one or more vessels before or during transfer to extraction at operation 108. Such a vessel is a form of a reservoir or buffer to provide a continuous supply of cannabis plant material for extraction, with some capacity to accommodate transfer/processing rate mismatch.

In some embodiments, an extraction solvent is used to transfer cannabis plant material from milling and/or decarboxylation to extraction. For example, milling operation 104 could include contacting cannabis plant material with an extraction solvent. The extraction solvent could be added before, during and/or after milling, and could be added by manually pouring the solvent into the milling machine and/or by using one or more components such as pipes, pumps and/or valves to transfer the extraction solvent into a milling machine. Any or all reduced size cannabis plant material produced at operation 104 could become at least partially dissolved and/or suspended in the extraction solvent, creating a solution and/or suspension of reduced size cannabis plant material. The fluidic properties of the extraction solvent could allow the cannabis plant material to flow from the milling machine to an extractor. As such, the extraction solvent could be considered a form of carrier or vehicle to carry any or all reduced size cannabis material from operation 104 to operation 108.

Using an extraction solvent to transfer reduced size cannabis plant material from a milling process to an extraction process could also have other uses. For example, certain waxes and/or other compounds in cannabis plant material could form a tacky residue that adheres to the inside of a milling machine during a milling process. When this residue builds up to a certain level, the efficiency and/or effectiveness of the milling process could be reduced, and the milling process might be interrupted or stopped to clean the milling machine. An extraction solvent that includes ethanol, for example, could function as a solvent for this residue, and could be used to clean the milling machine during a stoppage. In some embodiments, an extraction solvent is also or instead used to clean a milling machine during use, when the milling machine is actively milling cannabis plant material. Contacting the cannabis plant material with an extraction solvent during milling could wash waxes and/or other residues from a milling machine as the machine operates, potentially reducing the length and/or frequency of milling machine stoppages for cleaning.

In some embodiments, solvent exiting a milling machine contains dissolved and/or suspended reduced size cannabis plant material, flows into an extractor, and is used as an extraction solvent during solvent extraction. Additional solvent might or might not be added for extraction at 108. The solvent that carries the reduced size cannabis plant material into an extractor is sufficient to also perform extraction in some embodiments. However, in other embodiments, additional solvent is added during solvent extraction. Moreover, the solvent that carries the reduced size cannabis plant material to the extractor might not be used for extraction in all embodiments. For example, in an embodiment the reduced size cannabis plant material is filtered out of the solvent and transferred to the extractor, and fresh solvent and/or a different extraction solvent is then added and used for extraction.

Another potential use of solvent-based extraction is for performing decarboxylation. For example, if warm ethanol is used during extraction, then decarboxylation might occur during extraction. Thus, process 100 might not perform a separate decarboxylation operation at 106, 110 in the case that warm ethanol extraction is performed at operation 108, for example. If room temperature, cool, or super-cooled ethanol is used for extraction, then decarboxylation of a mixture of ethanol and cannabis plant material could be performed at operation 106. In some embodiments, operation 106 includes receiving a mixture of cannabis plant material and ethanol from operation 104. Decarboxylation could then be performed at operation 106 by heating this mixture in a flask or other container to induce decarboxylation.

Solvent-based extraction is one example of an extraction process. Mechanical extraction to separate trichomes from cannabis plant material, for example, may be used in some embodiments. Other embodiments could employ other types of extraction, and/or multiple types of extraction.

In some embodiments, the cannabis extract(s) produced by extraction at 106 are sent to operation 110 for decarboxylation, to produce decarboxylated cannabis extracts. For example, decarboxylation is performed at 110 in some embodiments in which decarboxylation is not performed at 106 and does not take place during extraction at 108. In the case that cannabis extract(s) are mixed with an extraction solvent, operation 108 could include heating the mixture in a flask or other container, for example, to induce decarboxylation. However, operation 110 need not be performed in all embodiments. Therefore, the cannabis extract(s) from operation 108 bypass operation 110 in some embodiments, and this is illustrated in FIG. 1.

Although not shown in FIG. 1, decarboxylation could be implemented following winterization at operation 112 and/or distillation at operation 114. However, some embodiments of the present disclosure relate to processes for producing cannabis products that do not involve decarboxylation after extraction. Furthermore, some processes might not include decarboxylation at all. For example, the production of cannabis-based vaporization substances might not include decarboxylation because decarboxylation could occur when a user vaporizes the vaporization substances.

An output of the extraction at operation 108, and/or an output of the decarboxylation at operation 110, could be a cannabis extract 120 that includes waxes and/or lipids. These waxes and/or lipids could include wax esters, glycerides, and/or unsaturated fatty acids that were extracted from cannabis plant material along with the cannabinoids and/or terpenes. Such waxes and/or lipids are also referred to as waxy ballast, and tend to hinder an extract from forming a refined liquid state. Extract 120, also referred to as a crude extract, a concentrate or a resin, is an example of a possible cannabis-containing end product of process 100, and is packaged and shipped to end users or other licensed producers in some embodiments. Even though an extract 120 is a cannabis product that is produced by the process 100 in some embodiments, the extract is further processed in some embodiments to produce cannabis-infused consumer products, for example. The following is a non-exhaustive list of examples of cannabis-infused consumer products that could be produced using a cannabis material or product as an ingredient:
Cannabis-infused beverages (beverages incorporating cannabinoid-containing substance(s) and which are intended to be consumed in the same manner as beverage drinks);
Cannabis-infused edibles (products incorporating cannabinoid-containing substance(s) and which are intended to be consumed in the same manner as food);
Cannabis-infused topicals (products that incorporate cannabinoid-containing substance(s) and which are intended to be used on external body surfaces, such as skin, hair, and/or nails);
Cannabis-infused mucoadhesive delivery systems (products that incorporate cannabinoid-containing substance(s) and which are intended to be used on mucosa body surfaces, such as mouth, anal, nasal and vaginal cavities); and
Cannabis-infused vaping oil (oil products incorporating cannabinoid-containing substance(s) and which are intended to be consumed in a vaping device, such as an electronic cigarette).

The winterization operation 112 is an operation to winterize a cannabis extract in the presence of a winterization solvent to obtain a winterized extract. Winterization is also be referred to as a secondary extraction, which extracts or removes one or more undesirable components from a cannabis extract. For example, in some embodiments winterization reduces the amount of, or even rids an extract of, any or all waxes and/or lipids, while retaining the more polar cannabinoid molecules.

A winterization solvent is incorporated with a cannabis extract such that the cannabis extract is in contact with the winterization solvent, and a mixture of cannabis extract and winterization solvent is formed. For example, in some embodiments a winterization solvent such as ethanol is mixed with the cannabis extract during operation 112. Other examples of winterization solvents include ethanol/water solutions and acetone, and other solvents are also possible. Additional solvent examples are provided elsewhere herein.

Also or alternatively, the cannabis extract could be mixed with the winterization solvent before operation 112. For example, ethanol is mixed with the cannabis extract during a prior extraction process and/or milling process in some embodiments.

Operation 112 also involves cooling the mixture of winterization solvent and cannabis extract in some embodiments. In other embodiments, the winterization solvent is cooled before it is mixed with the cannabis.

Although described herein primarily in the context of removal of undesirable components from a cannabis plant extract, winterization is also or instead used for other purposes in some embodiments. For example, in some embodiments winterization is used to flash-freeze cannabis plant material as an alternative to drying in pre-treatment before milling and/or as a pre-extraction processing operation after milling.

Winterization at 112 operates on a principle that undesirable component(s) in a cannabis plant extract have precipitation temperature(s) at which the undesirable component(s) precipitate out of solution in a winterization solvent but one or more cannabinoids and/or terpenes remain in solution. Therefore, the undesirable component(s) precipitate out of the solution while the cannabinoid(s) and/or terpene(s) remain dissolved. The undesirable component(s) can then be removed, by filtering for example, from the winterization solvent. In some embodiments, winterization also or instead releases any or all other trapped solvents, such as extraction solvents, from a cannabis extract.

Ethanol is polar solvent that is used as a winterization solvent in ethanol winterization or ethanolic precipitation, for example. However, other winterization solvents are also possible, and examples are provided herein. In some embodiments, ethanol winterization is used to separate and remove waxy ballast, and thereby purify a crude cannabis extract by removing or at least reducing such undesirable components as waxes and/or lipids.

A winterization chiller is used to carry out at least a portion of the winterization process at operation 112 in some embodiments. A winterization chiller cools a mixture of winterization solvent and cannabis extract to induce the precipitation of the undesirable component(s). Any of various types of refrigeration and/or freezing equipment, including refrigerators, freezers, and/or cooling or freezing tunnels which might be preferred for continuous processes, are suitable for implementation of a winterization chiller.

Removing the waxy ballast from the cannabis extract could include chilling a mixture of cannabis extract and winterization solvent to a temperature less than or equal to about 0° C., alternatively less than or equal to about −10° C., alternatively less than or equal to about −20° C., for a time period. The time period may be at least 1 hour, alternatively at least about 24 hours, alternatively at least about 48 hours, alternatively at least about 50 hours, alternatively at least about 72 hours. After the chilling period, the crude cannabis extract can be cold-filtered to remove the waxy ballast. For example, a filter with vacuum assist and/or pressure assist could be initially used to remove plant material that is insoluble, and secondly the crude extract could be run through syringe filters (for example, 0.45 and/or 0.2 micron filters), to filter out components such as remaining plant material and/or bacteria that may be present. In some embodiments, the winterization station 220 also includes one or more centrifuges to separate waxy ballast from the crude cannabis extract.

In some embodiments, a winterization station also includes one or more mixture vessels. Mixing during winterization may be useful, for example, to aid in avoiding or reducing clogging and maintaining flow as a crude cannabis extract is cooled.

In the example process 100, cannabis extract is transferred from operation 108 and/or from operation 110 to winterization at 112. In some embodiments, at least a portion of the transfer to the winterization process is automated. For example, a winterization chiller could be coupled to a decarboxylation device via a transfer mechanism that is configured for transferring the decarboxylated cannabis extract from the decarboxylation device to the winterization chiller. A winterization chiller could also or instead be coupled to an extractor via another transfer mechanism configured for transferring the cannabis extract from the extractor to the winterization chiller. The rate(s) of transfer, to a precipitation separator that is part of a winterization station for example, could be controlled to substantially match a rate of winterization in the winterization chiller and potentially avoid bottlenecks and/or cannabis material shortage at an input and/or output of the winterization at 112.

In some embodiments, cannabis extract is held in one or more vessels before transfer to a winterization chiller. Any such vessel could be considered a reservoir or buffer to provide a continuous supply of cannabis extract for winterization and at least some capacity to accommodate mismatch between transfer rate(s) and/or between transfer rate(s) and winterization rate.

In some embodiments, extraction solvent helps transfer cannabis extract from an extractor to a winterization chiller. For example, an extraction solvent could help reduce the viscosity of the extract and facilitate the flow of cannabis extract to the winterization chiller. The extraction solvent could be added before, during and/or after the extraction operation 108. For example, any or all ethanol that is added during the milling operation 104 and/or the extraction operation 108 could dissolve and/or suspend a cannabis extract, and thus reduce the viscosity of the cannabis extract.

An output of the winterization operation 112 is a cannabis extract 122 that has a lower amount or concentration of one or more undesirable components. In some embodiments, a winterized cannabis extract is substantially free of undesirable components such as waxes and/or lipids. Extract 122 is another example of a possible cannabis-containing end product of process 100, which is packaged and shipped to end users in some embodiments. Extract 122 could also or instead be transferred for further processing to produce cannabis-infused consumer products, examples of which are provided elsewhere herein.

Operation 114 involves distillation to purify, isolate and/or crystallize at least one cannabinoid from a cannabis extract. Distillation could include the use of a distillation column or other form of distiller, for example.

Inputs to distillation operation 114 could include cannabis extract transferred from operation 108, operation 110 and/or operation 112. In some embodiments, such transfers involve holding cannabis extract from operation 108, operation 110 and/or operation 112 in one or more vessels. Any or all of these vessels provide a form of reservoir or buffer, to enable a continuous supply of cannabis extract for distillation, and/or to accommodate at least some mismatch in rate(s) of flow and/or processing of cannabis material through the example process 100.

In some embodiments, at least a portion of the transfer of cannabis extract to distillation is automated. For example, a distiller could be coupled to an extractor, a decarboxylation device, and/or a winterization chiller via one or more transfer mechanisms configured for transferring cannabis extract from the extractor, decarboxylation device, and/or winterization chiller to the distiller. The rate(s) of transfer of cannabis extract are controlled in some embodiments to substantially match a rate of distillation in the distiller to potentially avoid buildup and/or shortage of cannabis material at an input and/or output of distillation at 114.

In some embodiments, a winterization solvent and/or an extraction solvent are used to help transfer cannabis extract to the distiller from a winterization chiller and/or an extractor. As noted above, ethanol or other appropriate solvent(s) could be used as both an extraction solvent and a winterization solvent, and therefore a solvent could help transfer cannabis extract from an extractor and/or a winterization chiller to the distiller. In such embodiments, one or more cannabinoids and/or terpenes are separated from a solvent by distillation, or the solvent is also or instead removed between any of operations 104, 106, 108, 112, 114 by filtering cannabis plant material and/or cannabis extract from the solvent, for example. In some embodiments, the solvent is removed into order to collect extracts 120, 122.

An output of the distillation at operation 114 is a distillate 124 that substantially consists of a single pure cannabinoid or a mixture of cannabinoids. For example, in some embodiments the output distillate includes a single cannabinoid having at least 90% purity, or at least 95% purity, or at least 98% purity, or at least 99% purity, or being almost 100% pure. Distillate 124 is another example of a possible cannabis-containing end product of process 100, and is packaged and shipped to end users in some embodiments. Distillate 124 could also or instead be further processed to produce cannabis-infused consumer products, for example, such as those described elsewhere herein.

The process 100 is at least partially automated in some embodiments, to provide a continuous process. Process automation in some embodiments allows one or more components, such as milling machines, decarboxylation devices, extractors, winterization chillers and/or distillers, to be operated more efficiently than in implementations that require a higher degree of human intervention. For example, in some embodiments automation enables cannabis material to be transferred to, from, and/or between operations at rates that are determined based on actual operating conditions or parameters at any of various locations or positions in a process or processing system. Another potential benefit of automation is to limit human involvement in a cannabis production process, thereby reducing the likelihood of human error and/or reducing contamination risk associated with cannabis material handling by personnel.

The foregoing description of process 100 illustrates that some compounds, such as ethanol or other solvent(s), could help facilitate a continuous process. Example uses of solvents include: use as a cleaning solvent for cleaning a milling machine at operation 104 (for example through high pressure steam processes), use as an extraction solvent at operation 108, use as a winterization solvent at operation 112, and use in helping perform decarboxylation at any or all of operations 106, 108, 110. A solvent is recovered and reused for multiple operations in some embodiments, potentially reducing the overall amount of solvent consumed relative to conventional processes.

In some embodiments, solvent is also involved in transferring cannabis materials. As an example, a solution and/or suspension of cannabis plant material and/or cannabis extracts in a solvent is used for transfers between any or all of operations 103, 104, 106, 108, 110, 112 and 114, in some embodiments. Since the solvent could confer at least some fluidic properties to the cannabis plant material and/or cannabis extracts, any of various fluidic components could be used to transfer the cannabis plant material and/or cannabis extracts between different processes. Examples of fluidic components include pipes, valves and pumps. These fluidic components could be automated, and therefore potentially reduce the need for manual operations in a process or processing system.

In some embodiments, the process 100 is made continuous at least in part by controlling one or more processes or processing devices/equipment so that there is a constant supply of input material for each of operations 103, 104, 106, 108, 110, 112 and 114. For example, operation 108 could include providing, and/or the use of, an extraction vessel that contains a cannabis plant extract in an extraction solvent. This extraction vessel could be a component of an extractor, or a separate vessel thereto.

Operation 108 could further include incorporating a cannabis plant material and a volume of extraction solvent into the vessel, and continuously withdrawing a portion of the extraction solvent containing the extract from the vessel. By continuously withdrawing a portion of the extraction solvent at a certain rate, operation 108 could substantially maintain a constant volume of cannabis plant material and extraction solvent in the vessel. In some embodiments, the volume is maintained within a certain range of a target volume, above a minimum volume, below a maximum volume, and/or at a volume relative to one or more thresholds. Substantially maintaining a constant volume in this example is one way, but not the only way, to implement a continuous process. Volume could vary to at least a certain degree, without vessel overflow or emptying for example, in a continuous process.

The rate at which the extraction solvent is withdrawn in this example could be predetermined, and/or sensors could actively adjust the rate to aid in maintaining a constant volume in the vessel. The withdrawn portion of the extraction solvent could transfer extract from the extraction vessel in operation 108 to winterization operation 112, for example. Also or alternatively, the withdrawn portion of the extraction solvent could transfer extract from the extraction vessel in operation 108 to distillation operation 114.

Although the embodiments described in relation to FIG. 1 primarily discuss the use of ethanol as an extraction solvent and a winterization solvent, this need not be the case in all embodiments. Other solvents could also or instead be used for cleaning a milling machine, serving as an extraction solvent during extraction, serving as a winterization solvent during winterization, and/or aiding in the transfer of cannabis plant material and/or cannabis extracts. Examples are disclosed elsewhere herein.

As noted above, the process 100 may be made continuous at least in part by controlling one or more processes or processing devices/equipment so that there is a constant supply of input material for each of operations 103, 104, 106, 108, 110, 112 and 114. In general, any operation, process, component, station, substation, or system may receive a continuous supply, continuous stream, continuous flow, or continuous transfer of an input cannabis material for processing and/or provide a continuous supply, continuous stream, continuous flow, or continuous transfer of an output processed cannabis material. Examples of input cannabis material for processing and output processed cannabis material are provided elsewhere herein. Various options in respect of operations, processes, components, stations, or substations that may be coupled to each other or otherwise receive cannabis material from or provide cannabis material to each other are also provided elsewhere herein. Cannabis material, which may take any of various forms dependent upon a particular operation, process, component, station, or substation, may be received in or received as a continuous supply, continuous stream, continuous flow, or continuous transfer. Cannabis material may also or instead be transferred in or transferred as a continuous supply, continuous stream, continuous flow, or continuous transfer to any operation, process, component, station, or substation.

Some aspects of the present disclosure relate to the integration of processes that are conventionally implemented as separate and distinct processes. For example, FIG. 1 illustrates possible integrated operations 130, 132, 134 using dashed rectangles. Integrated operation 130 includes any two or more of operations 103, 104, 106, 108. As such, any two or more of operations 103, 104, 106, 108 could be considered sub-operations of integrated operation 130. Similarly, integrated operation 132 includes any two or more of operations 103, 104, 106, 108, 110, 112, and integrated operation 134 includes any two or more of operations 103, 104, 106, 108, 110, 112, 114.

To implement integrated operations 130, 132, 134, several different systems and/or devices are integrated or combined. Examples of system integration include coupling different systems together, and providing a single system that performs two or more of the operations illustrated in FIG. 1.

One potential benefit of integrated operations such as those illustrated at 130, 132, 134 is reducing the number of manual steps in process 100. For example, in some embodiments integrated operations 130, 132, 134 provide automated operations that produce cannabis products with little to no human involvement.

Integrated operations 130, 132, 134 illustrated in FIG. 1 represent examples, and should not be considered limiting in any way. In general, integration is potentially applicable to any of operations 103, 104, 106, 108, 110, 112, 114 of FIG. 1 in any of a number of different combinations.

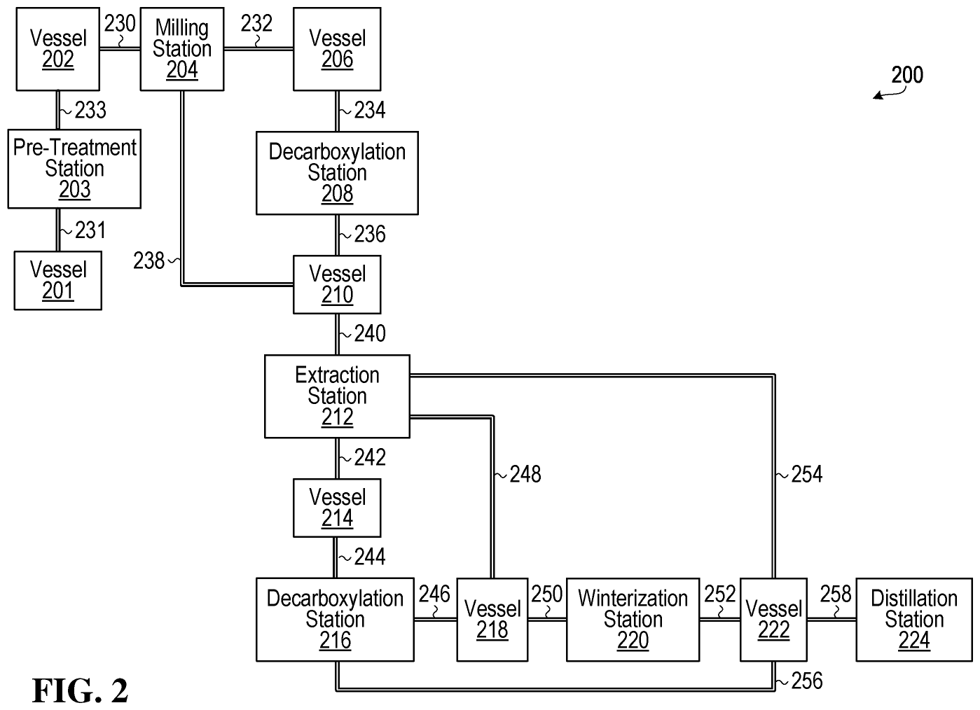

The foregoing description concentrates primarily on processing. Systems and devices for producing cannabis products are described in further detail below. FIG. 2 is a block diagram illustrating an example system 200 for producing cannabis products. In some embodiments, system 200 is used to implement processing consistent with process 100 of FIG. 1.

System 200 includes multiple processing stations 203, 204, 208, 212, 216, 220, 224, multiple vessels 201, 202, 206, 210, 214, 218, 222, and multiple transfer mechanisms 230, 231, 232, 233, 234, 236, 238, 240, 242, 244, 246, 248, 250, 252, 254, 256, 258 interconnecting vessels and processing stations.

Stations 203, 204, 208, 212, 216, 220, 224 could be discrete subsystems or devices for performing respective processes. However, as discussed in further detail herein, one or more of stations 203, 204, 208, 212, 216, 220, 224 could be integrated into a single station to perform multiple different processes. In a processing system that implements at least partially continuous processing and/or integrated processing stations, multiple stations 203, 204, 208, 212, 216, 220, 224 are co-located as part of a processing or production line for example. Embodiments in which processing stations 203, 204, 208, 212, 216, 220, 224 are at different locations, such as in different rooms or buildings or even at different sites, are also possible. As noted elsewhere herein, not all embodiments necessarily involve continuous or integrated processing.

The vessels 201, 202, 206, 210, 214, 218, 222 are provided to hold or store cannabis materials or cannabis products in system 200. For example, in an embodiment a vessel includes an intake or input port through which a cannabis material or cannabis product is received, an internal space for holding the cannabis material or cannabis product, and an outlet or output port through which the cannabis material or cannabis product is released or dispensed from the vessel. As used herein, a vessel is intended to denote any type of holding container in which a cannabis material or cannabis product is or could be contained. This includes holding containers that are used for storing cannabis materials or cannabis products before, during and/or after processing, as well as containers that store cannabis products for sale. Hoppers, bins, and tanks are examples of vessels that are suitable for use in a production setting such as system 200. Vessels could be constructed from one or more materials such as wood, paper, cardboard, plastic, glass, for example.

In some embodiments, vessels are sealed or sealable, to seal cannabis materials or cannabis products from their environment. Examples of vessel seals include caps, lids, and covers. Vessels could also or instead be sealed with one or more of: foil seals, heat seals, induction seals, and shrink wrap, for example. In some embodiments, seals are tamper-resistant or tamper-proof, as in the case of a tamper- proof induction seal, for example.

Vessels that are at least partially open to a surrounding environment are also possible.

The present disclosure is not limited vessels of any particular physical dimension(s). Vessels of any of a variety of different shapes, including cylindrical, rectangular, and/or triangular, for example, could be implemented in a processing system such as 200. Similarly, volume of a vessel is not limited in the embodiments described herein. A vessel could have a volume less than about 1L, less than about 5L, less than about 10L, less than about 20L, less than about 50L, less than about 100L, less than about 200L, less than about 500L, or less than about 1000L, for example.

In general, vessel characteristics are chosen or selected based on any of various processing system or processing criteria. In some embodiments vessel size is determined based on one or more of: physical space available to build a processing or production line, the type of cannabis material or cannabis product that is to be held or stored (cannabis plant material before milling occupies more space than reduced size cannabis plant material after milling for example), the amount of cannabis material or cannabis product that is to be held or stored (it may be desirable to store a larger amount of input cannabis material for a faster processing station than for a slower processing station, and/or to have a larger holding or storage capacity for processed cannabis material from a faster processing station than from a slower processing station, for example), and/or how cannabis materials or cannabis products are to be separated or packaged (for example, it might be necessary to maintain separation or trackability between different lots or amounts of cannabis materials or cannabis products in order to satisfy regulatory and/or other requirements, which in turn dictates a maximum vessel storage capacity at one or more stations in a processing system).

Other vessel characteristics such as material construction, type, and sealed or unsealed design, are also potentially determined or selected based on any of various criteria. Vessel construction and/or type could depend upon the type of cannabis material or cannabis product that is to be held or stored, and/or the particular transfer mechanism(s) or processing station(s) to which a vessel is to be coupled, for example. A sealed vessel might be preferred over a partially open or unsealed vessel for cannabis materials or cannabis products that are sensitive to environmental conditions and/or for longer term holding or storage above a time threshold such as beyond an expected processing time to complete a production run.

Other embodiments in which these and/or other criteria are taken into account in vessel design, and/or other aspects of processing system design, are also possible.

Transfer mechanisms 230, 231, 232, 233, 234, 236, 238, 240, 242, 244, 246, 248, 250, 252, 254, 256, 258 are provided to move cannabis materials or cannabis products throughout system 200. For example, a transfer mechanism could be used to move a cannabis material or cannabis product from one station or vessel to another station or vessel. Active transfer mechanisms and passive transfer mechanisms are possible.

An active transfer mechanism is powered or driven to impart motion to a cannabis material or cannabis product. An example of an active transfer mechanism is a pump, for a fluid or liquid-like cannabis material or cannabis product such as a cannabis extract and/or a cannabis material or cannabis product that is dissolved and/or suspended in a solvent or other liquid for example. Conveyors are another example of an active transfer mechanism, for solids such as cannabis plant material or milled cannabis plant material for example. Non-limiting examples of conveyors include conveyor belts, roller conveyors, vibrating conveyors, chain conveyors, bucket conveyors and screw or auger conveyors.

A gravity feed, from a hopper through a bottom or lower outlet for example, is illustrative of a passive transfer mechanism. A passive transfer mechanism enables transfer of a cannabis material or cannabis product, but does not itself induce motion to the cannabis material or cannabis product. A hollow structure that is capable of transporting matter is referred to generally herein as a pipe or conduit, and represents another example of a passive transfer mechanism. Pipes could be straight, bent or curved, for example. Pipes are also not limited to any particular cross-sectional shape or size. For example, circular, rectangular and triangular cross-sections are possible.

In some embodiments, a transfer mechanism includes both one or more active components and one or more passive components. As an example, in a transfer mechanism in which a pump forces a fluid through a conduit or pipe, the pump is an active component and the conduit or pipe is a passive component.

Other components are also provided in some embodiments of transfer mechanisms. For example, some embodiments include one or more valves to control the flow of cannabis materials or cannabis products into vessels, into pipes, out of vessels, and/or out of pipes.

Although transfer mechanisms and vessels are illustrated in FIG. 2 as separate components, this might not always be the case. In some embodiments, a transfer mechanism includes one or more vessels. For example, in an embodiment, transfer mechanism 232 includes vessel 206. A pipe and a vessel could even be integrated together as a combined, unitary component. Moreover, in another embodiment, transfer mechanisms 232, 234 and vessel 206 are part of the same transfer mechanism. Similar comments also apply to transfer mechanisms 230, 231, 233, 236, 238, 240, 242, 244, 246, 248, 250, 252, 254, 256, 258 and vessels 201, 202, 210, 214, 218, 222.

Embodiments in which a processing station or subsystem includes one or more vessels and/or one or more transfer mechanisms are also possible. In an embodiment, a transfer mechanism, such as a gravity feed or pipe for example, is built into or otherwise integrated with a processing station to provide at least part of an input or output transfer mechanism for the processing station.

Characteristics of transfer mechanisms are chosen or selected based on any of various processing system or processing criteria. In some embodiments transfer mechanism type, size, and/or transfer or driving capacity are determined based on one or more of: suitability for automation (an active transfer mechanism might provide more granular and/or reliable control than a passive transfer mechanism for example), physical space available to build a processing or production line, the type of cannabis material or cannabis product that is to be transferred (a conveyor for solids such as cannabis plant material or a pump for fluids or liquid-like cannabis materials or cannabis products for example), the amount of cannabis material or cannabis product that is to be transferred (larger pipe size, pump size, and/or conveyor size for moving larger amounts of cannabis material to and/or from a faster processing station that processes material faster than a slower processing station), and/or transfer speed (to transfer cannabis materials or cannabis products at a speed that is expected to achieve or at least be conducive to meeting target parameters for such conditions as settling, dissolution, precipitation, change in temperature, change in pressure, filtering, evaporation, and/or condensation during transfer, for example).

Other transfer mechanism characteristics such as material construction and/or shape, are also potentially determined or selected based on any of various criteria. Such criteria include those described above or elsewhere herein, and/or possibly others such as the particular vessel(s) or processing station(s) to which a transfer mechanism is to be coupled, for example.

Other embodiments in which these and/or other criteria are taken into account in transfer mechanism design, and/or other aspects of processing system design, are also possible.

Examples of systems, devices, or equipment to carry out pre-treatment, milling, decarboxylation, extraction, winterization, and distillation are provided above, with reference to these operations in FIG. 1. These examples are illustrative of possible options for implementing pre-treatment station 203, milling station 204, decarboxylation station 208 and/or 216, extraction station 212, winterization station 220, and distillation station 224 in FIG. 2. Additional and/or more detailed examples are also provided below.

In the example system 200, vessel 202 is provided to hold cannabis plant material that is used as a source material for the system. This cannabis plant material could be cannabis plant material 102 described above with reference to FIG. 1, for example. In some embodiments, harvested cannabis plant material is placed in vessel 201, transferred to pre-treatment station 203 by transfer mechanism 231, pre-treated at pre-treatment station 203, and then transferred to vessel 202 by transfer mechanism 233. Examples of pre-treatment operations and devices or equipment to perform such operations are provided elsewhere herein, including at least above with reference to pre-treatment at 103 in FIG. 1. Examples of pre-treatment operations and devices or equipment to perform such operations are provided elsewhere herein, including at least above with reference to pre-treatment at 103 in FIG. 1. Examples of transfer mechanisms are also provided elsewhere herein.

Vessel 202 is coupled to milling station 204 via transfer mechanism 230. In some embodiments, transfer mechanism 230 includes a conveyor that transfers cannabis plant material from vessel 202 to milling station 204 at a predetermined or variable transfer rate. In another embodiment, vessel 202 is a hopper that is mounted to or otherwise located above milling station 204, and transfer mechanism 230 includes a gravity feed from the hopper to an inlet or input port of the milling station.

Milling station 204 is provided to reduce the physical size of the cannabis plant material. In some embodiments, the milling station is coupled to receive pre-treated cannabis plant material from the pre-treatment station 203 and reduce size of the pre-treated cannabis plant material.

Milling station 204 includes a milling machine or shredder in an embodiment. In a milling machine that includes rotating blades driven by a motor, for example, the rotating blades could be at least partially enclosed by a chamber or a tube. In the case of a milling machine that includes a chamber, cannabis plant material could be added into the chamber, milled using the rotating blades, and then removed from the chamber. In the case of a milling machine that includes a tube for enclosing the rotating blades, cannabis plant material could be fed into one end of the tube, be milled by the rotating blades, and then flow from the other end of the tube. Using a tube to enclose rotating blades in a milling machine could allow for a continuous milling process, in which cannabis plant material is continuously fed into one end of the tube, flows through the tube during milling, and is continuously collected from the other end of the tube.

Feeding cannabis plant material into a milling machine could include a gravity feeding process as noted herein. An angle of a milling machine chamber or tube relative to vertical could influence the rate at which cannabis plant material passes through the milling machine, and thus could also influence how finely the cannabis plant material is milled. For example, orientating the tube of a milling machine with its axis close to vertical might cause cannabis plant material to pass through the milling machine relatively quickly in comparison to a tube that is oriented at a greater angle from vertical. A more vertical orientation could therefore result in the production of relatively large particles of cannabis plant material. In some embodiments, an infeed transfer mechanism angle and/or a milling chamber or tube angle is used as a control parameter, and is adjustable, manually or automatically in response to sensed output particle size, to provide control over a feeding or flow rate of cannabis plant material into a milling station 204.

Decarboxylation station 208 is coupled to receive reduced size cannabis plant material from milling station 204. As illustrated in FIG. 2, decarboxylation station 208 is coupled to milling station 204 via transfer mechanisms 232, 234, and vessel 206.

Transfer mechanisms 232, 234 are configured for transferring reduced size cannabis material from milling station 204 to decarboxylation station 208 through vessel 206. In this context, such configuration for transferring reduced size cannabis material refers to ability of a transfer mechanism to transfer reduced size plant material. Transfer mechanism 232 collects or otherwise receives reduced size cannabis plant material from an output of a milling machine at milling station 204, and deposits the reduced size cannabis plant material into vessel 206 through an inlet or input port. Vessel 206 is implemented to hold the reduced size cannabis material that is produced by the milling station 204 before transfer of the reduced size cannabis material for further processing. Transfer mechanism 234 collects or otherwise receives reduced size cannabis plant material from an output of vessel 206, and deposits the reduced size cannabis plant material into decarboxylation station 208 through an inlet or input port.

For example, transfer mechanisms 232, 234 could include respective conveyors to carry reduced size cannabis plant material from milling station 204 to vessel 206 and from vessel 206 to decarboxylation station 208. In some embodiments, reduced size cannabis plant material from milling station 204 is in a fluid or liquid-like state (for example, if the reduced size cannabis plant material is mixed with a solvent) and the transfer mechanisms 232, 234 include pipes and possibly one or more pumps.

Decarboxylation station 208 performs decarboxylation, an example of which is shown as operation 106 in FIG. 1 and described above. In an embodiment, decarboxylation station 208 includes a heating element to increase the temperature of the cannabis plant material and induce decarboxylation. In some embodiments, a decarboxylation heater is used to heat the cannabis plant material. In other embodiments, a heated container at decarboxylation station 208 used to heat a mixture of cannabis plant material and a solvent. Other examples of equipment provided at a decarboxylation station 208 in some embodiments include a CSTR and a PFR. A choice between a decarboxylation device, a heated container, and/or other equipment at decarboxylation station 208 is made in some embodiments based on such factors as whether the reduced size cannabis plant material received from milling station 204 is mixed with a solvent.

Extraction station 212 is also coupled to receive reduced size cannabis plant material from milling station 204. Two options for reduced size cannabis plant transfer from milling station 204 to extraction station 212 are shown in FIG. 2. One transfer path is through transfer mechanisms 238, 240 and vessel 210, and another transfer path is through transfer mechanisms 232, 234, 236, 240, vessels 206, 210, and decarboxylation station 208. Some embodiments include only one of these transfer paths, and other embodiments include both transfer paths. In some embodiments with both of these transfer paths, two vessels are provided at 210, including one to hold reduced size cannabis plant material from milling station 204 and another to hold decarboxylated reduced size cannabis plant material from decarboxylation station 208.

Transfer mechanisms 232, 234 and vessel 206 are described above, and transfer mechanisms 236, 238, 240 and vessel 210 are implemented in the same manner or similarly in some embodiments. Vessel(s) 206 hold the reduced size cannabis plant material from milling station 204 and/or decarboxylated reduced size cannabis plant material from decarboxylation station 208 before transfer to extraction station 212.

In some embodiments, transfer mechanisms 236, 240 include respective conveyors to receive and carry reduced size cannabis plant material from decarboxylation station 208 to extraction station 212 via vessel 210. Similarly, transfer mechanisms 238, 240 include conveyors in some embodiments to receive and carry reduced size cannabis plant material from milling station 204 to extraction station 212 via vessel 210. In some embodiments, extraction station 212 is in fluid communication with or fluidly connected to milling station 204 and vessel 210. For example, any or all of transfer mechanisms 232, 234, 236, 238, 240 could include pipes to carry solutions and/or suspensions of reduced size cannabis material in a solvent. Milling station 204 could be configured for contacting cannabis plant material with this solvent.

For example, ethanol and/or another solvent could be added to a milling machine in milling station 204 while the milling machine is operating. The solvent could dissolve and/or suspend reduced size cannabis plant material produced by the milling machine, and carry this cannabis plant material through a pipe in transfer mechanism 238. Vessel 210 could hold the solution/suspension of the cannabis plant material, and transfer mechanism 240 could transfer the solution/suspension to extraction station 212. When the ethanol reaches extraction station 212, it could be used as an extraction solvent. Therefore, the solvent that transfers reduced size cannabis plant material from milling station 204 to extraction station 212 could be an extraction solvent.

Extraction station 212 performs extraction to obtain a cannabis extract including at least one cannabinoid. The extraction station could be configured to obtain the cannabis extract by performing mechanical extraction on the reduced size cannabis plant material, for example. In some embodiments, extraction station 212 is configured to obtain the cannabis extract by extracting reduced size cannabis plant material with an extraction solvent. Extraction station 212 could be configured for contacting the reduced size cannabis plant material with the extraction solvent during a fluid extraction process, for example. In some embodiments the extracting involves a warm solvent extraction process that further causes decarboxylation of the at least one cannabinoid.

In an embodiment, extraction station 212 includes an extractor or an extraction vessel in which the solvent extraction process is performed. Such an extractor could include an inlet or input port to receive cannabis plant material, an interior space to hold the received cannabis plant material, and an outlet or output port for the produced cannabis extract. An extractor could further include or be coupled to flasks, pots, valves, channels, coils and/or pumps to transfer extraction solvent into contact with the cannabis plant material. One or more pumps, heaters, chillers, and/or valves could adjust or control any of a number of parameters in the extractor, including temperature and/or pressure, to perform extraction.

Other types of extraction are also or instead implemented at the extraction station 212 in other embodiments.

Decarboxylation station 216 is one of the stations in system 200 that is coupled to receive cannabis extract from extraction station 212 in the example shown. As illustrated in FIG. 2, decarboxylation station 216 is coupled to extraction station 212 via transfer mechanisms 242, 244, and vessel 214. Decarboxylation station 216 could be implemented in the same or a similar manner as decarboxylation station 208, for example.

Winterization station 220 is another station coupled to receive cannabis extract from extraction station 212. Extraction station 212 and winterization station 220 are coupled via transfer mechanisms 248, 250, and vessel 218. Winterization station 220 could also or instead receive cannabis extract via decarboxylation station 216 and vessel 218 or a separate vessel. Decarboxylation system 216 and winterization station 220 are coupled via transfer mechanisms 246, 250, and vessel 218 in the example shown. Transfer mechanisms 242, 244, 246, 248, 250 are configured for transferring (possibly decarboxylated) cannabis extract from extraction station 212 to winterization station 220. Vessel 214 and/or vessel(s) 218 are provided to hold cannabis extract from extraction station 212 before transfer to winterization station 220.

In some embodiments, winterization station 220 is in fluid communication with or fluidly connected to extraction station 212 and vessel(s) 218. For example, any or all transfer mechanisms 242, 244, 246, 248, 250 could include pipes to fluidly connect winterization station 220 and extraction station 212. An extraction solvent, which could have been used during fluid extraction at extraction station 212, could transfer cannabis extract from extraction station 212 to winterization station 220. For example, ethanol containing dissolved and/or suspended cannabis extract from the extraction station 212 could flow through transfer mechanisms 248, 250 and a vessel 218 to winterization station 220.

Winterization station 220 is provided to winterize a cannabis extract. The cannabis extract could include one or more cannabinoids and an undesirable component such as waxes and/or lipids. To perform winterization, winterization station 220 could include a chiller and/or a precipitation separator, for example. In some embodiments, winterization station 220 is configured for contacting a cannabis extract with a winterization solvent. The winterization solvent could be added when the cannabis extract reaches the winterization station 220. However, an extraction solvent from extraction station 212, such as ethanol, could also be used in winterization station 220 as a winterization solvent. As such, additional winterization solvent might, but need not necessarily, be added at winterization station 220.

In some embodiments, winterization station 220 is configured to perform a continuous winterization process on a cannabis extract using a precipitation separator that includes a cooling path. The cannabis extract could include an extraction solvent such as ethanol, and the cooling path could be a pipe or other channel in which the cannabis extract is cooled.

Extraction station 212 could continuously supply the cannabis extract to the precipitation separator, where the cannabis extract could pass through the cooling path at a predefined flow rate. The flow rate could be controlled using valves at the inlet and/or outlet of the cooling path, for example. Pumps could also or instead be used to help control the flow rate. In some embodiments, the cannabis extract is gravity fed through the cooling path, and therefore such parameters as any one or more of angle of the cooling path with respect to vertical, shape of the cooling path, size of the cooling path, and the drag exerted on the cannabis extract by the cooling path, could be adjusted to help control the flow rate. For example, the drag exerted on the cannabis extract by the cooling path could be adjusted by changing the width or cross-sectional area of the cooling path.

The cannabis extract is cooled as it passes through the cooling path, to induce precipitation of the undesirable component(s). In some embodiments, heat is extracted from the cooling path using a heat exchanger that could be implemented in any of a number of different ways. For example, the cooling path could be located in a chiller to reduce the temperature of the cooling path. The cooling path could also or instead be in contact with a coolant, such as liquid nitrogen, to reduce the temperature of the cooling path. Controlling the rate of heat extraction from the cooling path in relation to the flow rate could bring the cannabis extract passing through the cooling path to a temperature that is below a precipitation temperature of the undesirable component(s) to induce the precipitation of the undesirable component(s). The undesirable component(s) might be repeatedly or continuously removed from the cooled cannabis extract as it flows through the cooling path using one or more filters and/or membranes, for example.

Distillation station 224 is yet another station coupled to receive cannabis extract from extraction station 212. Extraction station 212 and distillation station 224 are coupled via transfer mechanisms 254, 258 and a vessel 222. Decarboxylation station 216 and distillation station 224 are coupled via transfer mechanisms 256, 258, and a vessel 222. Winterization station 220 and distillation station 224 are coupled via transfer mechanisms 252, 258, and a vessel 222. The various transfer mechanisms 242, 244, 246, 248, 250, 252, 254, 256, 258 in the possible transfer paths between extraction station 212 and distillation station 224 are configured for transferring cannabis extract from the extraction station, possibly through other stations, to the distillation station. Vessel 214, vessel(s) 218, and/or vessel(s) 222 could hold (possibly decarboxylated and/or winterized) cannabis extract from extraction station 212 before transfer to distillation station 224. In some embodiments, distillation station 224 is in fluid communication with or fluidly connected to extraction station 212 and a vessel 222. For example, any or all transfer mechanisms 242, 244, 246, 248, 250, 252, 254, 256, 258 could include pipes to fluidly connect distillation station 224 and extraction station 212. An extraction solvent, which could have been used during fluid extraction at extraction station 212, could transfer cannabis extract from extraction station 212 to distillation station 224.

Distillation station 224 is also coupled to receive winterized cannabis extract from winterization station 220 in the example system 200. Transfer mechanisms 252, 258 are configured for transferring the winterized cannabis extract from winterization station 220 to distillation station 224. A vessel 222, which is separate from vessel(s) that are coupled to extraction station 212 and/or decarboxylation station 216 in some embodiments, is provided to hold the winterized cannabis extract from winterization station 220 before transfer to distillation station 224.

In some embodiments, distillation station 224 is in fluid communication with or fluidly connected to winterization station 220 and vessel 222. For example, either or both of transfer mechanism 252, 258 could include a pipe to carry a winterized cannabis extract. The winterized cannabis extract could be an oil-like product that flows freely. A winterization solvent could also or instead transfer the winterized cannabis extract from winterization station 220 to the distillation station 224. The winterization solvent could have been mixed with the cannabis extract at winterization station 220. The winterization solvent could have also functioned as an extraction solvent and/or a solvent for cleaning a milling machine for example, and therefore the winterization solvent could have been added upstream of winterization station 220.

Distillation station 224 could include distillation column or other form of distiller, for example, to purify at least one cannabinoid in a cannabis extract that is received at the distillation station. A distiller could include one or more flasks, heating elements, pumps, and cooling channels. The cooling channels could be coupled to refrigeration units and/or coolant, for example. In some embodiments, extract that is received at distillation station 224 is held in an input flask of the distiller and heated to evaporate at least a portion of the extract, which could include cannabinoids and/or terpenes, for example. The vaporized cannabinoids and terpenes flow into one or more cooling channels. Vacuum pumps, for example, could induce the flow of cannabinoids and terpenes into the cooling channel(s). The cannabinoids and terpenes condense at different points in these cooling channels based on their respective condensation temperatures, and are separated into different collection flasks or containers. This type of distiller is only one example, and other embodiments include distillers that are configured and/or operated differently.

The numbers and configurations of components illustrated in system 200 are provided by way of example. Other embodiments could have more, fewer and/or different numbers of components configured in a similar or different manner. For example, other systems could be implemented without one or more of vessels 202, 206, 210, 214, 218, 222, and with stations directly coupled together via a single transfer mechanism. In some embodiments, certain stations are not coupled to each other using a transfer mechanism, and instead cannabis product is manually moved from one station to another. More or fewer stations than shown could be implemented. For example, only one of decarboxylation stations 208, 216 might be used in other systems, and some systems might not have any decarboxylation systems at all.

A separation station is illustrative of another station that is provided in some embodiments. For example, a separation station could be coupled to receive a cannabis extract directly or indirectly from an extraction station such as 212, to separate at least one cannabinoid and/or terpene from the cannabis extract. In another embodiment, a separation station is coupled to receive winterized cannabis extract directly or indirectly from a winterization station such as 220, to separate at least one cannabinoid and/or terpene from the winterized cannabis extract. According to another embodiment, a separation station is coupled to receive a distillate directly or indirectly from a distillation station such as 224, to further purify at least one cannabinoid and/or terpene.

Some embodiments include a pre-treatment station to pre-treat cannabis plant material, and one or more other processing stations are coupled to receive pre- treated cannabis plant material from the pre-treatment station. The milling station 204 is coupled to receive pre-treated cannabis plant material from the pre-treatment station and reduce size of the pre-treated cannabis plant material in some embodiments.

It should therefore be appreciated that FIG. 2, like other drawings herein, is intended solely as an illustrative example. In some embodiments, a system could include only some of the components that are shown, such as a first station (204 for example) to reduce physical size of a cannabis plant material, and a second station (212 for example) coupled to receive reduced size cannabis plant material from the first station to obtain from the reduced size cannabis plant material a cannabis extract including at least one cannabinoid and/or terpene. Examples of inter-station couplings to enable a station, in this example the second station, to receive an output from another station, in this example the first station, include: the stations being in fluid communication with each other, and the stations being coupled together via a transfer mechanism such as a conveyor and/or a pipe and possibly one or more vessels. The second station may be coupled to receive a continuous supply of reduced size cannabis plant material, for example, in any of various embodiments disclosed herein.

Additional stations are also provided in some embodiments. According to one such embodiment, a winterization station such as 220 is coupled to receive the cannabis extract from the second station, to winterize the cannabis extract. Fluid communication and transfer mechanisms are examples of inter-station couplings suitable for transferring cannabis extract to a winterization station. For example, in any of various embodiments disclosed herein, the winterization station may be coupled to receive a continuous supply of the cannabis extract from the second station.

Other stations that are also or instead provided in some embodiments include, for example: a distillation station such as 224 coupled to receive winterized cannabis extract from a winterization station to purify the at least one cannabinoid and/or terpene, a distillation station coupled to receive the cannabis extract from an extraction station to purify the at least one cannabinoid and/or terpene, and a decarboxylation station such as 208 and/or 216, coupled to receive and decarboxylate reduced size cannabis plant material and/or cannabis extract. Again, fluid communication and transfer mechanisms are examples of inter-station couplings suitable for transferring reduced size cannabis plant material and/or cannabis extract from one processing station to another. In any of various embodiments disclosed herein, the distillation station may be coupled to receive a continuous supply of the winterized cannabis extract from the winterization station or a continuous supply of cannabis extract from the second station, for example.

A separation station is provided in some embodiments, in addition to or instead of a distillation station such as 224, to isolate or separate at least one cannabinoid and/or terpene from an extract or solution. Membrane filtration or separation, in which an extract or solution is passed through one or more membranes, is one example implementation of isolation or separation.

In the above examples, one or more other stations are provided in addition to a first station to reduce physical size of a cannabis plant material and a second station to obtain from the reduced size cannabis plant material a cannabis extract including at least one cannabinoid and/or terpene. Other subsets of stations are also possible. In another embodiment, a system includes a first station such as 212 to process a cannabis plant material to obtain a cannabis extract including at least one cannabinoid and a second station, coupled to receive the cannabis extract from the first station, to purify the cannabis extract. The cannabis extract is continuously transferred from the first station to the second station and/or received by the second station as or in a continuous supply of the cannabis extract in some embodiments.

A system may also include a transfer mechanism, coupled to the first station and to the second station, to continuously transfer at least a portion of the cannabis extract from the first station to the second station. The first station may be configured to obtain the cannabis extract by processing the cannabis plant material with an extraction solvent, and the transfer mechanism may be configured to transfer at least the portion of the cannabis extract to the second station in at least a portion of the extraction solvent. In some embodiments, the first station is configured to obtain the cannabis extract by performing mechanical extraction on the cannabis plant material.

Examples of a second station in this context of purifying the cannabis extract include a winterization station such as 220 to process the cannabis extract and obtain a winterized extract, a distillation station such as 224 to process the cannabis extract and obtain the at least one cannabinoid and/or terpene, and a separation system to obtain or further purify the at least one cannabinoid and/or terpene. Both a winterization station 220 and a distillation station 224, coupled to receive and process winterized extract from the winterization station to obtain the at least one cannabinoid and/or terpene, are provided in some embodiments. Similarly, some embodiments include a distillation station, other embodiments include a separation station, and some embodiments include both a distillation station and a separation station.

A system may include a transfer mechanism, coupled to a winterization substation and to a distillation substation, to transfer winterized extract to the distillation substation. In a system with a winterization substation and a separation substation, a transfer mechanism may be coupled to the winterization substation and to the separation substation, to transfer winterized extract to the separation substation. A system with a separation substation and a distillation substation may include a transfer mechanism, coupled to the separation substation and to the distillation substation, to transfer distillate to the separation substation. Such transfers of cannabis material between substations (or stations), like other transfers of cannabis material herein, may be continuous to provide a continuous supply, continuous stream, continuous flow, or continuous transfer.

Other variations are also possible. For example, the first station may include an extraction vessel to hold the cannabis extract in an extraction solvent, and a transfer mechanism coupled to the extraction vessel and configured to continuously withdraw a portion of the extraction solvent containing the cannabis extract from the extraction vessel so as to substantially maintain at least a minimum volume of plant material and extraction solvent in the extraction vessel. The transfer mechanism may be configured to continuously withdraw the portion of the extraction solvent containing the cannabis extract from the extraction vessel so as to substantially maintain a constant volume of plant material and extraction solvent in the extraction vessel, as in other embodiments disclosed herein.

In an extraction vessel embodiment, the second station may include a winterization substation coupled to the transfer mechanism, to receive the withdrawn portion of the extraction solvent containing the cannabis extract. As in other embodiments, the winterization substation may be configured to contact the extract with a winterization solvent, for example.

A distillation substation may be coupled to the transfer mechanism, to receive the withdrawn portion of the extraction solvent containing the cannabis extract, and in some embodiments the second station include a separation substation in fluid communication with the distillation substation. A transfer mechanism may be coupled to the separation substation and to the distillation substation, to transfer a distillate from the distillation substation to the separation substation.

A separation substation may instead be coupled to the transfer mechanism, to receive the withdrawn portion of the extraction solvent containing the cannabis extract.

Multiple substation embodiments are possible in conjunction with an extraction vessel. For example, the second station may include a distillation substation in fluid communication with a winterization substation that is coupled to receive the withdrawn portion of the extraction solvent containing the cannabis extract. A transfer mechanism may be coupled to the winterization substation and to the distillation substation, to transfer winterized extract to the distillation station, as in other embodiments. In some embodiments, a separation substation is in fluid communication with the winterization substation, and a transfer mechanism may be coupled to the winterization substation and to the separation station, to transfer winterized extract to the separation station.

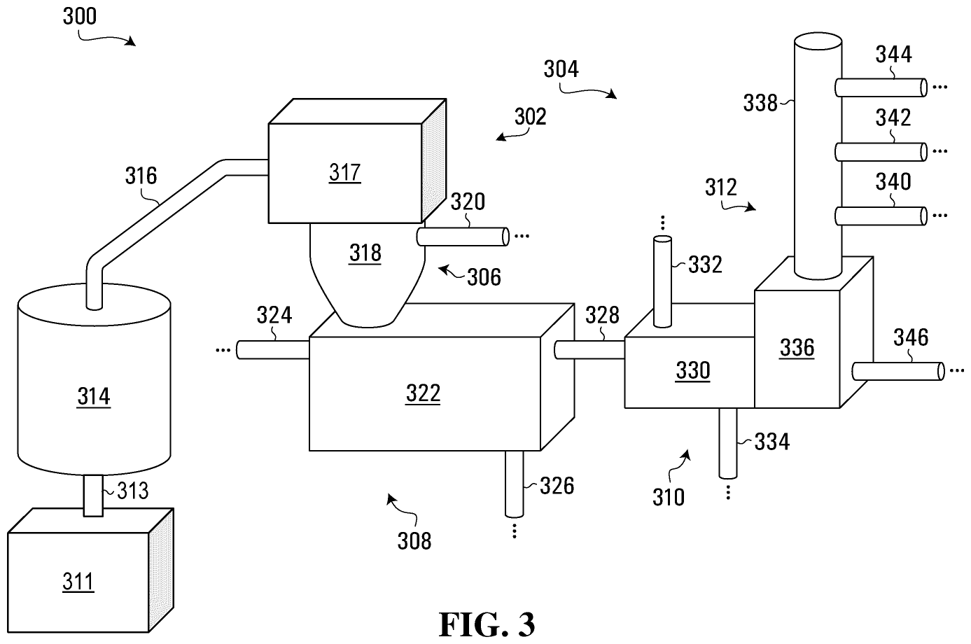

As noted herein, some aspects of the present disclosure relate to integration of devices, equipment, or systems that are conventionally implemented separately. Such integration is another example of a way in which processing stations are coupled together in some embodiments, to enable stations to receive outputs from other stations and/or provide inputs to other stations. FIG. 3 is a block diagram illustrating an integrated system 300 for the production of cannabis products according to one such embodiment.

System 300 includes a pre-treatment station 311, a vessel 314, a conveyor 316, an extraction station 302 and a purification station 304. A pipe 328 couples extraction station 302 and purification station 304 in the example shown.

Extraction station 302 includes a pre-treatment substation 317, a milling substation 306 and an extraction substation 308. The term “substation” refers to a discrete part or component of an integrated processing station. A substation has a distinct function within the integrated processing station. For example, an integrated processing station includes multiple substations that each perform a distinct process or operation. These substations are integrated together within the integrated processing station to produce a unitary or cohesive result.

In extraction station 302, pre-treatment station 317 is coupled to milling substation 306, which includes a milling machine 318 and a pipe 320 that is coupled to the milling machine. Extraction substation 308 includes an extraction vessel 322, and two pipes 324, 326 that are coupled to the extraction vessel. Extraction station 302 processes a cannabis plant material to obtain a cannabis extract including at least one cannabinoid and/or terpene. Specifically, a pre-treatment substation 317 is provided in some embodiments to pre-treat cannabis plant material, milling substation 306 is provided to reduce the size of the cannabis plant material, and extraction substation 308 is provided to obtain the cannabis extract from the reduced size cannabis plant material. As illustrated in FIG. 3, extraction substation 308 is coupled to receive the reduced size cannabis plant material directly from milling substation 306, by gravity feed in the example shown. The milling machine 318 is mounted above an inlet or input port of the extraction vessel 322, and feeds milled cannabis material into the extraction vessel. A manual transfer, for example, is not needed to transfer the reduced size cannabis material to extraction substation 308. In at least this sense, extraction substation 308 and milling substation 306 are considered to be integrated together. Similarly, milling machine 318 is coupled to receive pre-treated cannabis plant material from pre-treatment substation 317 by gravity feed in the example shown. The pre-treatment substation 317 is mounted above an inlet or input port of the milling machine 318 and feeds pre-treated cannabis plant material into the milling substation 306. In at least this sense, pre-treatment substation 317 and milling substation 306 are considered to be integrated together. Extraction station 302 in the example shown integrates pre-treatment substation 317, milling substation 306, and extraction substation 308.

Purification station 304 includes a winterization substation 310 and a distillation substation 312. Winterization substation 310 includes a chiller 330, and two pipes 332, 334 that are coupled to the chiller. Distillation substation 312 includes a heating element 336, a pipe 346 that is coupled to the heating element, a distillation column 338 that is also coupled to the heating element, and three pipes 340, 342, 344 that are coupled to the distillation column. Purification station 304 is coupled to receive the cannabis extract from extraction station 302, to purify the cannabis extract by winterizing and/or distilling the cannabis extract in the example shown. One or more other purification substations, including a separation system for example, are also or instead provided in other embodiments.

Winterization substation 310 processes the cannabis extract to obtain a winterized extract. Distillation substation 312 processes the winterized extract to obtain at least one cannabinoid and/or terpene. As illustrated in FIG. 3, distillation substation 312 is coupled to receive winterized extract directly from winterization substation 310. In at least this sense, winterization substation 310 and distillation substation 312 are considered to be integrated together.

The structure and/or function of any or all of substations 317, 306, 308, 310, 312 could be similar to any or all of stations 204, 212, 220, 224 of FIG. 2, for example.

In some embodiments, system 300 is used to process a cannabis plant material at extraction station 302 to obtain a cannabis extract, and continuously transfer at least a portion of the cannabis extract to purification station 304. Cannabis plant material is received and stored in vessel 314. Pre-treatment station 311 is coupled to vessel 314 by a transfer mechanism 313 in the example shown, and illustrates that pre- treatment is not necessarily implemented only in an integrated processing station such as 302 or only in a separate processing station such as 311. Pre-treatment, and similarly other processing operations, could be implemented in one or more integrated processing stations, in one or more separate processing stations, or both in one or more integrated processing stations and in one or more separate processing stations.

Conveyor 316 is coupled to vessel 314 to transfer the cannabis plant material to extraction station 302. In an arrangement as shown in FIG. 3, conveyor 316 is provided to receive the cannabis plant material from vessel 314 and convey the cannabis plant material to pre-treatment subsystem 317. Pre-treated cannabis plant material is dropped into the top of milling machine 318 after pre-treatment is complete.

In some embodiments, a container of solvent (not shown) is coupled to pipe 320, and solvent is transferred to milling machine 318 through the pipe 320. Any such added solvent mixes with the cannabis plant material being milled. As discussed elsewhere herein, the solvent could help to actively clean milling machine 318, and/or help to transfer milled material out of the milling machine. The transfer of cannabis plant material and/or solvent into milling machine 318 could be continuous and/or automated. Any additives for other operations, such as pre-treatment at 311 and/or 317 could similarly be supplied to pre-treatment station 311 and/or pre-treatment substation 317 through one or more pipes and/or other transfer mechanisms.

An output of milling machine 318 is directly coupled to an input of extraction vessel 322 to allow reduced size cannabis plant material to enter the extraction substation 308 directly after milling. A mesh filter, for example, could be provided between milling machine 318 and extraction vessel 322 to help ensure that only cannabis plant material smaller than a predefined size can enter the extraction vessel. Any solvent added to milling machine 318 could also help wash reduced size cannabis material from the milling machine into extraction vessel 322. The transfer of reduced size cannabis plant material and/or solvent into extraction vessel 322 could be continuous and/or automated.

In an embodiment, solvent fluid extraction is performed in extraction vessel 322, and examples of such extraction are provided herein. Any solvent that flows into extraction vessel 322 from milling machine 318 could act as an extraction solvent. Extraction solvent could also or instead be added from a container of extraction solvent (not shown) to extraction vessel 324 through pipe 324. In some embodiments, pipes 320, 324 are coupled to the same solvent container.

In some embodiments, an extraction station is configured to obtain the cannabis extract by performing mechanical extraction on cannabis plant material.

Waste that is produced in extraction vessel 322 could be separated from the cannabis extract and removed from extraction vessel 322 through pipe 326. The removal of waste is periodic in some embodiments, and continuous in other embodiments. Waste removal is automated in some embodiments. For example, a brush or filter could periodically or continuously sweep through extraction vessel 322 to catch or trap waste material and separate the waste material from the cannabis extract. Pipe 326 could be connected to a container (not shown) to store the waste from extraction vessel 322. In some embodiments, an active transfer mechanism is provided to remove the waste from extraction vessel 322, to deal with waste that does not flow freely for example.

Cannabis extract is transferred from the extraction vessel 322 to chiller 330 through pipe 328. This transfer could include transferring cannabis extract in an extraction solvent. For example, a mixture of cannabis extract and extraction solvent could flow through pipe 328 and into chiller 330. The flow of cannabis extract could be controlled by valves at the outlet of extraction vessel 322 and/or the inlet of chiller 330. Pipe 328 could also or instead include one or more valves, one or more pumps, and/or one or more vessels to aid in controlling the flow of cannabis extract.

In some embodiments, the flow of cannabis extract from extraction vessel 322 is continuous. The rate of flow of material out of extraction vessel 322 could be controlled to substantially match a rate of flow of material into the extraction vessel. For example, the rate of flow into extraction vessel 322 could be equal to the sum of the rate of reduced size cannabis material and extraction solvent entering the extraction vessel from milling machine 318, and the rate of extraction solvent entering the extraction vessel from pipe 324. The rate of flow out of extraction vessel 322 could be equal to the sum of the rate of waste material exiting the extraction vessel through pipe 326, and the rate of cannabis extract and/or extraction solvent exiting the extraction vessel through pipe 328. Matching a rate of flow into and out of extraction vessel 322 could help prevent bottlenecks forming during the operation of system 300, which could otherwise result in stoppages. One or more vessels could also be useful in accommodating mismatch between flow rate(s) and/or between flow rate(s) and extraction rate.

Chiller 330 performs a winterization process on the output from extraction vessel 322, which is a mixture of cannabis extract and extraction solvent in some embodiments. Winterization solvent, which could be the same as or different from the extraction solvent, could be added to chiller 330 through pipe 332 and mixed with the cannabis extract. The pipe 332 is coupled to a source of winterization solvent, which could be the same vessel to which one or both of the pipes 320, 324 are coupled, or a different vessel for example.

The cannabis extract flows through chiller 330 during winterization. A heat exchanger in chiller 330, for example, cools the mixture of cannabis extract and winterization solvent to induce precipitation of one or more undesirable components such as waxes. A brush or filter periodically or continuously sweeps through chiller 330 to catch or trap the undesirable components and separate the undesirable component(s) from the winterized extract in some embodiments. In other embodiments, the chiller 330 also or instead includes other elements or devices for removal of undesirable component(s), such as any one or more of: one or more precipitation separators, one or more centrifuges, and one or more filters. One or more mixture vessels are also provided in the chiller 330 in some embodiments.

Pipe 334 is provided in the example system 300 to enable the undesirable component(s) to be removed from chiller 330, and to possibly deposit the undesirable component(s) in a container (not shown) for example. Although such component(s) are undesirable in a cannabis extract, any or all component(s) may have other uses, and therefore need not necessarily be discarded.

An output of chiller 330 is directly coupled to an input of heating element 336 to allow the winterized cannabis extract to enter the heating element directly after winterization. A filter, for example, could be provided between chiller 330 and heating element 336 to help prevent any undesirable component(s) such as precipitated waxes, for example, from flowing into the heating element 336. The winterized cannabis extract could flow to heating element 336 in a continuous stream, and the rate of flow of winterized cannabis extract could substantially match a rate of flow of cannabis extract into chiller 330 to avoid bottlenecks and/or stoppages in the chiller, for example. One or more vessels could be provided at an inlet, an outlet, and/or inside of the chiller 330 to help accommodate mismatch between flow rate(s) and/or between flow rate(s) and winterization rate.

Heating element 336 is provided to initiate a distillation process. For example, heating element 336 could be coupled to a container of winterized cannabis extract, to heat the winterized cannabis extract. Cannabinoids and terpenes in the winterized cannabis extract evaporate and flow into distillation column 338. Vacuum pressure, for example, could induce the flow of cannabinoids and/and terpenes into distillation column 338. Distillation column 338 is cooled, by the ambient atmosphere and possibly with the aid of a heat exchanger, to cool the vaporized cannabinoids and/and terpenes. Within distillation column 338, the cannabinoids and terpenes condense at different points based on their relative condensation temperatures. Pipes 340, 342, 344 capture and separate the different cannabinoids and/or terpenes based on where they condense in distillation column 338. Pipes 340, 342, 344 are shown by way of example, and more or fewer pipes or distillate collectors could be coupled to a distillation column in other embodiments. Furthermore, other devices, in addition to or different from pipes 340, 342, 344, could be used to capture and separate different cannabinoids and/or terpenes in a distillation column.

Some components of the winterized cannabis extract received by heating element 336 might not be vaporized. Pipe 346 represents an example of a device or element to enable such components, whether or not considered waste, to be removed from heating element 336. In some implementations, one or more undesirable components could be vaporized by heating element 336 and collected by one or more pipes 340, 342, 344. For example, a solvent could be vaporized and collected. This solvent might be reused in one or more stations of system 300.

System 300 is provided by way of example, and other integrated systems for producing cannabis products are also possible. For example, not all processing stations are necessarily integrated. In an embodiment, a first station such as 302 to process a cannabis plant material to obtain a cannabis extract including at least one cannabinoid and/or terpene is implemented with a first substation such as 306 to reduce size of the cannabis plant material and a second substation such as 308, coupled to receive reduced size cannabis plant material from the first substation, to obtain the cannabis extract from the reduced size cannabis plant material. In other embodiments, a first station includes a pre-treatment substation such as 317 to pre-treat cannabis plant material, and an extraction substation such as 322, coupled to receive pre-treated cannabis plant material from the pre-treatment substation, to obtain the cannabis extract from the pre-treated cannabis plant material. Another example of an integrated processing station is as shown in FIG. 1, with three substations including a pre-treatment substation such as 317 to pre-treat cannabis plant material; a first substation such as 318, coupled to receive pre-treated cannabis plant material from the pre-treatment substation, to reduce size of the pre-treated cannabis plant material; and a second substation such as 322, coupled to receive reduced size cannabis plant material from the first substation, to obtain the cannabis extract from the reduced size cannabis plant material.

Such an integrated processing station could be implemented on its own or in conjunction with other stations that might or might not be integrated. In the embodiment shown in FIG. 3, a second station 304 is also an integrated processing station.

A second station that includes both a winterization substation 310 to process the cannabis extract and obtain a winterized extract, and a distillation substation 312 to process winterized cannabis extract and obtain the at least one cannabinoid and/or terpene, is also an illustrative example. Only one or the other of these substations is provided in other embodiments. Other stations that are implemented in conjunction with an integrated purification station such as 304 need not necessarily also be integrated processing stations.

As another example, in some embodiments a second station includes a separation substation to process the cannabis extract and obtain the at least one cannabinoid and/or terpene. Another embodiment of an integrated second station includes a winterization substation and a separation station, coupled to receive the winterized extract from the winterization substation, to process the winterized extract and obtain the at least one cannabinoid and/or terpene. In a further embodiment, an integrated second station includes a distillation station and a separation station, coupled to receive from the distillation substation a distillate that includes the at least one cannabinoid and/or terpene, to process the distillate and further purify the at least one cannabinoid and/or terpene.

Similar embodiments of a purification station are also contemplated, including the following examples: a purification station that includes a separation station, coupled to receive cannabis extract from an extraction station, to separate at least one cannabinoid and/or terpene in the cannabis extract and obtain the at least one cannabinoid and/or terpene from the cannabis extract; a purification station that includes a winterization station and a separation station, coupled to receive winterized extract from the winterization station, to separate at least one cannabinoid and/or terpene in the winterized cannabis extract and obtain the at least one cannabinoid and/or terpene from the winterized cannabis extract; and a purification station that includes a distillation station and a separation station, coupled to receive from the distillation station a distillate that includes at least one cannabinoid and/or terpene, to further purify the at least one cannabinoid and/or terpene by separating the at least one cannabinoid and/or terpene in the distillate.

FIGS. 1 to 3 provide high-level views of example cannabis processing and production or processing systems. More detailed examples are also provided below.

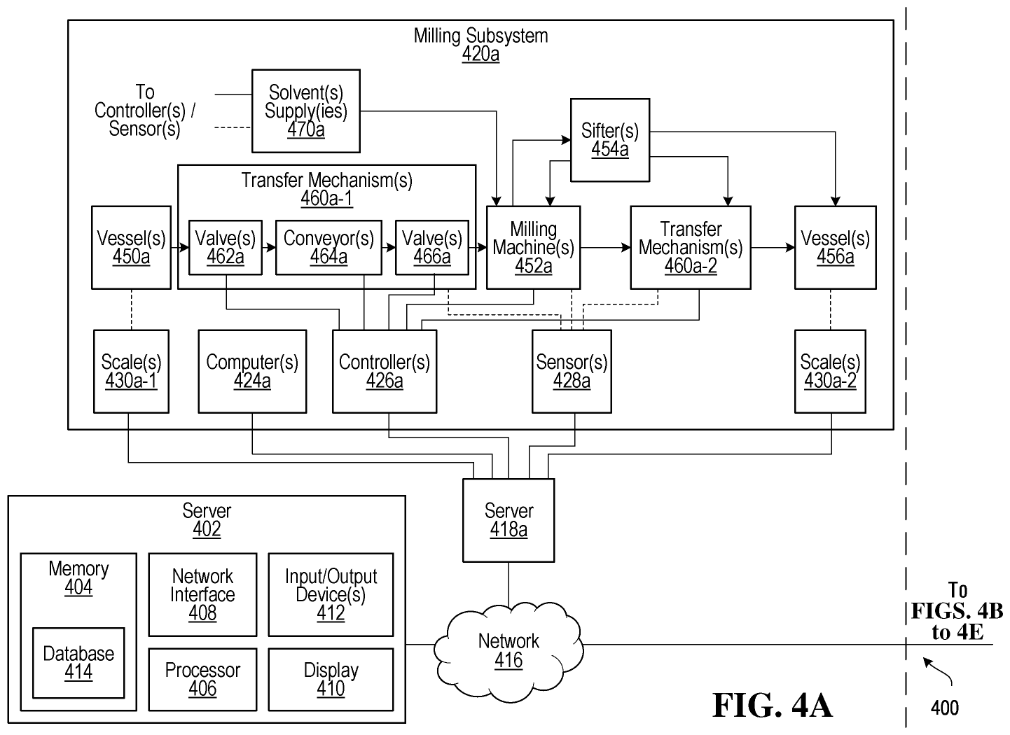

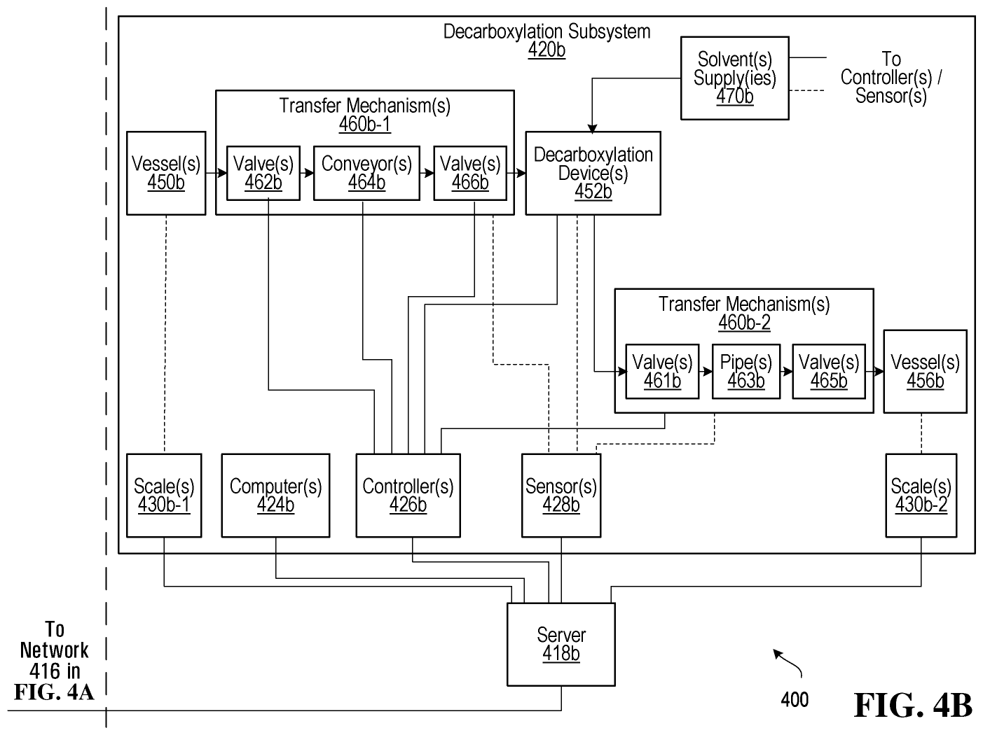

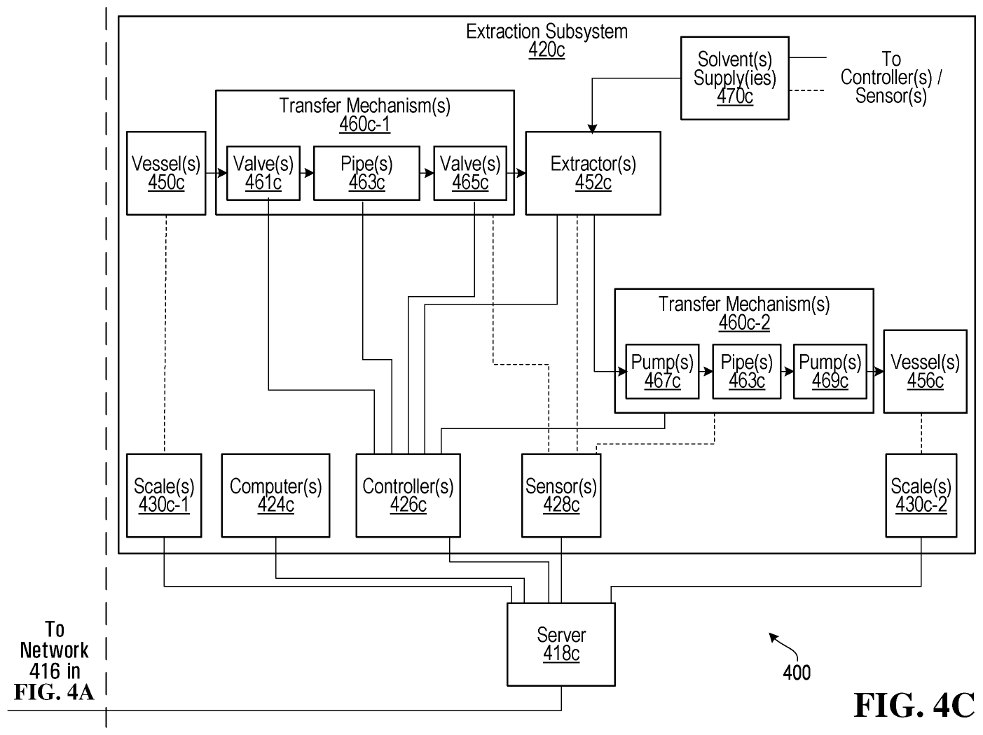

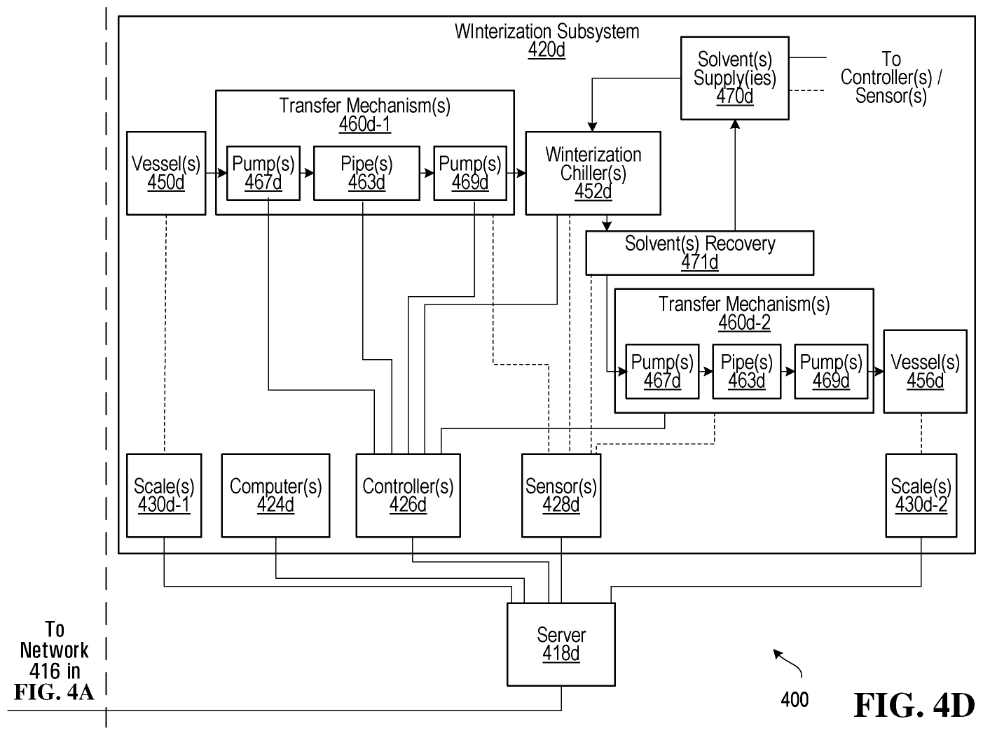

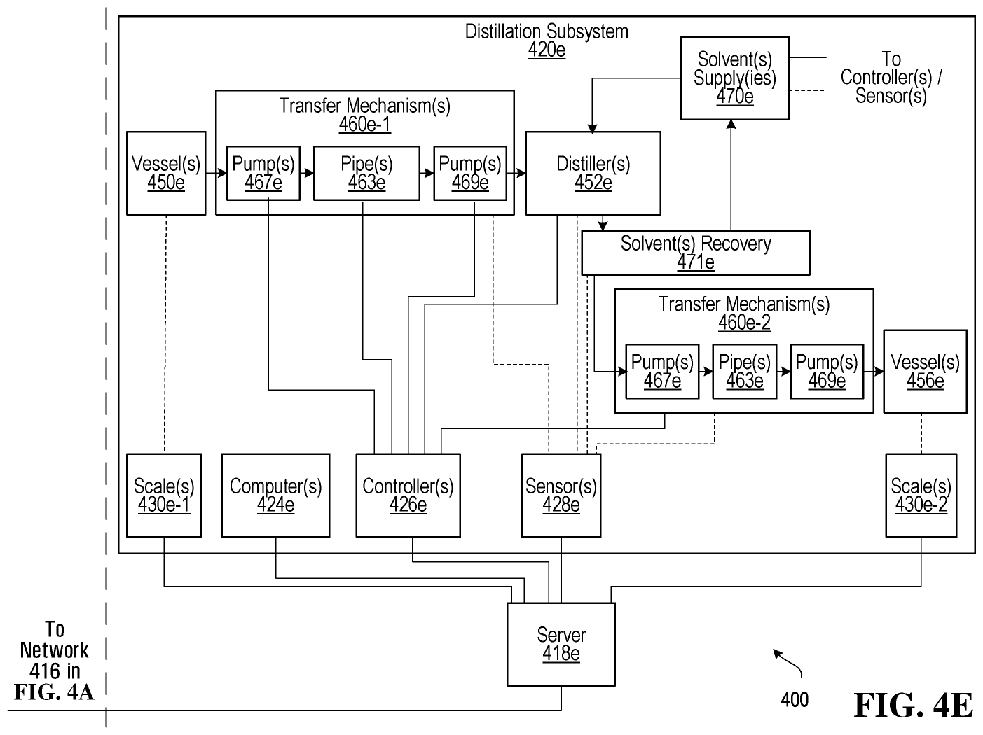

FIGS. 4A-4E are block diagrams illustrating an example automated cannabis material processing system. This example system 400 includes various constituent subsystems as example implementations of stations that are described above and/or elsewhere herein.

The system 400 includes, as shown in FIGS. 4A-4E, respectively, a milling subsystem 420a, a decarboxylation subsystem 420b, an extraction subsystem 420c, a winterization subsystem 420d, and a distillation subsystem 420e. Each of these example subsystems is described in detail herein. A processing system could be implemented in conjunction with other systems or subsystems, such as any one or more of: a cultivation and harvest system, a plant part separation system, a waste destruction system, a fresh plant material processing system, a drying system, an oil formulation system, a packaging system, a sterilization system, a testing system, and a shipping system as disclosed in Canadian Patent Application No. 3,033,404, filed on Feb. 11, 2019, for example. Canadian Patent Application No. 3,033,404 is incorporated in its entirety herein by reference. A drying system is an example of a pre-treatment systems, and more generally one or more pre-treatment systems are provided in some embodiments. Examples include not only drying systems, but also freezing systems, dewaxing systems, and digestion systems.

The system 400 also includes a server 402. The server 402 includes a memory 404 storing a database 414, a processor 406, a network interface 408, a display 410, and one or more input/output (I/O) devices 412. In some embodiments, these server components are interconnected to each other by an internal bus and/or other type(s) of connection(s).

The memory 404 could be or include one or more memory devices, such as one or more solid state memory devices, and/or one or more memory devices that use movable or even removable storage media. The database 414 could be formatted or otherwise provided in the memory 404 to store information that is related to processing of cannabis material and/or cannabis product(s) produced by such processing. For example, in some embodiments the database 414 stores records, parameters, measurements and/or other information for such purposes as processing/production history recording, auditing, and/or tracking by an inventory control system (ICS).

The processor 406 is implemented in some embodiments by one or more processors that execute instructions stored in the memory 404. Example implementations of the processor 406 include implementations, in whole or in part, using dedicated circuitry, such as an application specific integrated circuit (ASIC), a central processing unit (CPU), and/or a programmed field programmable gate array (FPGA) for performing any of various operations of the processor, for example.

The network interface 408 is an example of an input-output device, and enables communications between the server 402 and other devices or systems over a network 416. The particular structure of the network interface 408 is implementation- dependent, and may vary between embodiments that support different types of connections and/or communication protocols, for example. The network interface 408 could enable communications over wired and/or wireless connections. In general, a network interface includes a physical interface such as a port, connector, or other component to interface with a communication medium, and a receiver and/or transmitter to process received signals and/or process transmit signals for transmission. A transceiver is an example of a component that includes both a receiver and a transmitter, and could be implemented in the network interface 408.

The display 410 is another example of an input-output device, to allow users such as system operators to view any or all information stored in the database 414 and/or to otherwise interact with the server 402 and/or other components of the system 400. For example, in some embodiments the display 410 enables a user to access and show a record for a cannabis product and/or process. The display 410 could also or instead allow a user to view the current status of any or all systems within the system 400, including information regarding which systems or devices are currently in use, the processes these systems or devices are performing, and/or the operator(s) using the systems or devices, for example. Any of various types of displays could be implemented at 410, including touchscreen displays that also enable user input.

Other I/O devices 412 could also or instead be provided. For example, one or more user input devices that allow a user to manually input information, actions and/or requests could be provided. Examples of user input devices include keyboards, computer mice, touchscreens, buttons, dials and switches. The I/O devices 412 could also or instead include one or more output devices, such as output ports for exporting data stored in the database 414. Other types of I/O devices are also contemplated. An access card scanner, for example, could provide security and access control for the server 402.

In some embodiments, the server 402 itself does not include user I/O devices such as a display 410 or user input devices for receiving inputs from a user. User interaction with the server 402 could be through one or more separate components such as one or more workstations that communicate with the server 402 through local connections with the server and/or network connections through the network 416. Such workstations could be identical to or similar in structure to the server 402, but might not locally store the database 414, for example.

The network 416 could be or include any of various types of network equipment implementing any of various type(s) of network(s). In some embodiments, the network 416 includes a corporate network of a cannabis processor or cannabis producer. The network 416 could also or instead include the internet. The particular type(s) of networks(s) in a system such as 400 could be implementation-dependent. In some embodiments, the server 402 is located at a corporate office, and at least some of the subsystems 420a-420e are located remotely from the server 402. At least the remotely-located subsystems could connect or otherwise communicate with the server 402 through the internet, whereas co-located subsystems that are at the same location as the server 402 could connect or otherwise communicate with the server through a local area network (LAN) or other type(s) of local network(s).

The network 416 is connected to or otherwise in communication with multiple servers 418a, 418b, 418c, 418d, 418e, shown in FIGS. 4A-4E, respectively. Network/server communications could be provided, for example, using physical connections such as cables and/or wires, and/or using wireless connections or channels, such as WIFI™ connections, Bluetooth™ connection, and/or longer-range wireless communications.

The servers 418a-418e could be generally similar in structure to the sever 402, but there could be at least operational, and/or possibly structural, differences between servers. For example, the servers 418a-418e could be involved in maintaining the database 414 at the server 402 by sending system-related information through the network 416 to the server 402, but the servers 418a-418e might not locally store a complete copy of the database 414.

In some embodiments, the servers 418a-418e relay information from other devices to the server 402. Information could also or instead be stored on the servers 418a-418e. Although the servers 418a-418e could be distributed throughout the system 400 as shown, this might not always be the case. Two or more of the servers 418a- 418e, for example, could be co-located. Although illustrated separately in FIGS. 4A-4E, in some embodiments two or more of the servers 418a-418e are implemented using a single server. At least some of the subsystems 420a-420e could be connected to or otherwise in communication with the network 416 without an intervening server 418a- 418e. One or more components of a system 420a-420e could communicate with the network 416 without necessarily traversing a server in connecting to the network. A subsystem component could also or instead communicate with the network 416 through some other type of communication equipment or device that does not necessarily implement a server.

The milling subsystem 420a includes one or more computers 424a, one or more controllers 426a, one or more sensors 428a, and one or more scales 430a-1, 430a-2. These components are each connected to the server 418a in the example shown. Connections between these components and the server 418a could include wired and/or wireless connections, through any of various types of interfaces. Each component that is connected to or otherwise in communication with the server 418a includes an interface compatible with an interface that is provided at the server. The particular type(s) of interface(s) provided at the subsystem components and the server 418a would be dependent upon the type(s) of connection(s) and/or communication protocol(s) to be supported.

In some embodiments, one or more computers such as 424a are implemented for such purposes as enabling users or operators to manually enter data, otherwise interact with the system 400, and/or control system devices. For example, a computer 424a could store entered data and/or transmit entered data to the server 418a, which could store and/or forward the data to the server 402. A computer 424a could also or instead enable an operator to access data, in the database 414 for example, and output an indication of that data on a display screen or other output device. Examples of computers include desktop computers, laptop computers, tablet computers and other electronic devices. In general, a computer 424a could be similar in structure to a server 402 and/or 418a, but need not necessarily store the database 414. Depending on implementation, a computer 424a might or might not include a network interface. In a server-based implementation as shown in FIG. 4A, for example, a computer 424a could include an interface that might or might not be a network interface but is compatible with an interface provided at the server 418a.

In some embodiments, one or more controllers such as 426a are implemented to control any or all of various types of devices or equipment. A controller could be integrated within a controlled device or equipment, or be separate from the controlled device or equipment as shown in FIG. 4A. Controllers could be implemented, for example, using hardware, firmware, one or more components that execute software stored in one or more non-transitory memory devices. Microprocessors, ASICs, FPGAs, and Programmable Logic Devices (PLDs) are examples of processing devices that could be used to execute software. In some embodiments, a controller 426a includes a processor and computer readable storage in the form of one or more memory devices.

A controller 426a could store, receive, and/or otherwise obtain control settings, and control one or more devices or equipment to run according to those settings. For example, a controller 426a could be programmable by operators, through a computer 424a and/or through a user interface of the controller for example. A programmable controller 426a could access, download, or otherwise determine, or be programmed with, control settings from the database 414. In some embodiments, a controller 426a records control settings and/or other information in the database 414. Information that is used by and/or obtained by a controller 426a could be locally stored, by the controller and/or another component of the milling subsystem 420a for example, and/or transmitted to the server 418a for local storage and/or transmission to the server 402.

In some embodiments, one or more sensors 428a are implemented to measure or otherwise determine any or a variety of parameters involved in processing cannabis material. These parameters, and possibly other information such as the time at which measurements were taken, could be recorded in the database 414. Examples of sensors, any one or more of which could be implemented at 428a, include the following: flow sensors to measure or otherwise determine flow rate(s) of cannabis material(s) or cannabis product(s) through the milling subsystem(s), and milling parameter sensors to measure or otherwise determine any of various milling parameters such as milling machine operating conditions and/or current size of milled cannabis plant material.

Sensor readings or measurements could be locally stored, by a sensor 428a and/or another component of the milling subsystem 420a for example, and/or transmitted to the server 418a for local storage at the server 418a and/or transmission to the server 402.

In some embodiments, one or more scales such as 430a-1, 430a-2 are implemented to weigh cannabis materials, cannabis products, and/or vessels such as holding containers, for example. Although two sets of scale(s) are shown at 430a-1, 430a-2, in some embodiments a milling subsystem includes only one set of one or more scales. Different vessels could be transferred to the same weighing station with one set of scales, for example. One or more sets of scales are inline in a processing system in some embodiments, not only in a milling subsystem but also or instead in other subsystems.

Scales could include, for example, electronic scales that are in communication with or otherwise able to access the database 414. When an electronic scale measures the weight of a cannabis material, a cannabis product, and/or a vessel, for example, the scale could automatically transmit this weight to the server 418a, where it could be recorded and/or transmitted to the server 402. Non-electronic scales could also or instead be implemented, and the weights measured by these scales could be manually entered into the system 400 using a computer 424a, l for example.

A description of a weight measured by a scale 430a-1, 430a-2 could also be recorded in the system 400. The description could include information regarding the current stage of production of a cannabis material, cannabis product and/or vessel when the cannabis material, cannabis product, or vessel was weighed. An operator could manually enter this description into an electronic scale 430a-1, 430a-2 or a computer 424a, for example, which could then transmit the description to the server 418a and/or server 402. A description of a measured weight could also or instead be inferred by a computer 424a and/or a server 402, 418a. For example, an electronic scale 430a-1, 430a-2 could be associated with a specific step or operation during cannabis processing or a specific device or equipment in a cannabis processing system, and a description of the weights measured by that scale could therefore be predefined in a computer 424a and/or a server 402, 418a. For example, in some embodiments a certain scale 430a-1 is only used to measure the weight of vessels containing cannabis plant material before milling, and a computer 424a and/or a server 402, 418a automatically associates any or all weights measured by that scale with the pre-milling stage of processing or production.

A scale 430a-1, 430a-2 receives control information and/or other information in some embodiments. For example, a scale 430a-1, 430a-2 could be controlled to record a weight only when a vessel is in proper position for weighing. In some embodiments, a controller sends a control signal to a scale 430a-1, 430a-2 to trigger a measurement. Such a controller could be integrated with a scale 430a-1, 430a- 2, or be separate from the scale as shown by way of example at 426a. Measurement could also or instead be manually initiated or triggered by an operator, through a user interface of a scale 430a-1, 430a-2 or another component that is connected to or otherwise in communication with the scale.

Weight measurements, and/or possibly other information that is determined or otherwise obtained by or from a scale 430a-1, 430a-2, could be locally stored by the scale and/or another component of the milling subsystem 420a for example, and/or transmitted to the server 418a for local storage at the server and/or transmission to the server 402.

The milling subsystem 420a further includes one or more cannabis plant material vessels 450a, one or more milling machines 452a, one or more sifters 454a and one or more reduced size plant material vessels 456a. The vessels 450a, 456a could include any of various types of container, and different container types could be used for cannabis plant material at 450a and reduced size milled cannabis plant material at 456a. The vessel(s) 450a could contain cannabis flower and/or trim from plant part separation, and/or dried cannabis plant material from a drying process, for example.

Transfer mechanism(s) 460a-1, 460a-2 are also shown in FIG. 4A, and as an example the transfer mechanism 460a-1 is shown as including valve(s) 462a, 466a and one or more conveyors 464a. The valve(s) 462a, 466a are examples of flow control devices that control flow of cannabis plant material from the vessel(s) 450a to the milling machine(s) 452a. Such flow control is provided in other ways in some embodiments, by controlling a speed of the conveyor(s) 464a for example.

Examples of conveyors, and other types of transfer mechanisms such as transfer mechanisms that include one or more vessels, are provided elsewhere herein. Valve(s) such as those shown at 462a, 466a and in other drawings are intended to represent devices that are controllable to at least open and close to permit (when open) and block (when closed) flow of material in a processing system. The valve(s) 462a, 466a are provided to enable flow control for cannabis material. One or more valves could also or instead be provided to enable flow control for other materials, such as solvents. Examples of valves include ball valves, gate valves, butterfly valves, and check valves. Some valves are open-close controllable, and others enable more granular or graduated control to open or restrict passage of materials to different degrees. Different types of valves could be implemented, for example, depending on the type(s) of material(s) for which flow control is to be provided. In some embodiments, different types of valves are provided to enable flow control for solid materials and materials that include liquids, such as solutions and/or suspensions.

In some embodiments, one or more solvents are added to a milling machine 452a. For example, one or more solvents are added during milling of cannabis plant material in some embodiments to aid in cleaning of the milling machine(s) 452a, during or after milling to aid in transferring milled cannabis plant material out of the milling machine(s), and/or after milling to aid in cleaning the milling machine(s). In the milling subsystem 420a, such solvent(s) are added to the milling machine(s) 452a from one or more solvent supplies 470a. A solvent supply 470a could be coupled to or otherwise in communication with one or more controllers 426a and/or one or more sensors 428a to control and/or monitor supply of solvent(s) to the milling machine(s) 452a. Solvent transfer between the solvent supply(ies) 470a and the milling machine(s) 452a, and/or other components of the milling subsystem 420a, could be through one or more transfer mechanism(s) that have not been shown in FIG. 4A to avoid congestion in the drawing. Such transfer mechanism(s) could be active or passive, and include one or more flow control devices such as valves in some embodiments.

Some interconnections in FIG. 4A are in solid lines and others are in dashed lines. In FIGS. 4A-4E, solid lines are intended to represent wired or wireless connections for communications between components. Dashed lines are intended to indicate that components interact with each other or are related or associated in some way, but are not necessarily in communication with or coupled to each other. By way of example, the scale(s) 430a-1 could weigh the vessel(s) 450a, but this does not necessarily mean that scale(s) would be in communication with the vessel(s), or that the scale(s) would necessarily remain physically coupled to the vessel(s) after weighing.

In an embodiment, a vessel (or each vessel) 450a is weighed using the scale(s) 430a-1, to quantify inputs to the milling subsystem 420a. The cannabis plant material in the vessel(s) 450a is then transferred to the milling machine(s) 452a through the transfer mechanism(s) 460a-1. Examples of a milling machine 452a are provided elsewhere herein, and also include milling equipment to mill the cannabis plant material and/or one or more grinders to grind the cannabis plant material. One or more of the controller(s) 426a could be connected to or otherwise in communication with the milling machine(s) 452a to control the milling machine(s). One or more sensor(s) 428a could similarly be connected to or otherwise in communication with the milling machine(s) 452a, to measure one or more parameters and/or otherwise monitor one or more properties of a milling process or equipment.

Milled, reduced size cannabis plant material is then transferred to one or more vessel(s) 456a through the transfer mechanism(s) 460a-2.

In some embodiments, milled plant material is also or instead transferred to one or more sifters 454a. The sifter(s) 454a include one or more filters or screens to sift the milled cannabis plant material and separate it based on particle size. In some embodiments, the sifter(s) 454a are used for mechanical extraction, to produce kief and/or other extract(s) for example. Output(s) from the sifter(s) 454a are transferred to one or more vessel(s) 456a, and/or possibly returned to the milling machine(s) 452a for further milling. Cannabis plant material transfer between the sifter(s) 454a and either or both of the milling machine(s) 452a and the vessel(s) 456a could be through one or more transfer mechanism(s), which have not been shown in FIG. 4A to avoid congestion in the drawing. Such transfer mechanism(s) could be active or passive, and include one or more flow control devices such as valves in some embodiments. In some embodiments, a sifter 454a is coupled to or otherwise communicates with one or more controller 426a and/or one or more sensors 428a.

In the milling subsystem 420a, the scale(s) 430a-2 weigh the vessel(s) 456a. Weights as measured by the scale(s) 430a-2 could be used, for example, to reconcile input cannabis plant material with total output milled cannabis plant material, and/or otherwise to maintain desired and/or required records of cannabis material during processing.

Other devices or equipment such as barcode readers and/or other types of scanners could be implemented to obtain information for record-keeping and/or reporting. As an example, in some embodiments information is read from barcode labels on vessels by one or more barcode readers and recorded. Other types of information instead of or in addition to barcodes and other types of readers or scanners instead of or in addition to barcode readers are possible. Such features relating to information collection, recording, and/or reporting apply not only to a milling subsystem but also or instead to other subsystems.

Referring now to FIG. 4B, an example decarboxylation subsystem 420b includes one or more computers 424b, one or more controllers 426b, one or more sensors 428b, and one or more scales at 430b-1 and/or 430b-2. These components are connected to or otherwise in communication with the server 418b. Implementation options for all of these components are described herein, at least above with reference to FIG. 4A. Although two sets of scale(s) are shown in at 430b-1, 430b-2, in some embodiments a decarboxylation system includes only one set of one or more scales.

The decarboxylation subsystem 420b further includes one or more source material holding vessels 450b, one or more decarboxylation devices 452b and one or more decarboxylated material vessels 456b. The vessels 450b, 456b could include any of various types of container, and different container types could be used for source cannabis material and decarboxylated cannabis material. The source cannabis material vessel(s) 450b could contain cannabis flower and/or trim from plant part separation, dried cannabis plant material from a drying process, and/or milled cannabis plant material from a milling process, for example. In some embodiments, the source cannabis material vessel(s) 450b are or at least include the same vessel(s) as shown at 456a in FIG. 4A.

Transfer mechanism(s) 460b-1, 460b-2 are also shown in FIG. 4B. As examples, the transfer mechanism 460b-1 is shown as including valve(s) 462b, 466b and a conveyor 464b, and the transfer mechanism 460b-2 is shown as including valve(s) 461b, 465b and one or more pipes 463b as another example of a component to transfer or carry cannabis material between processing equipment, components, or subsystems. The valve(s) 462b, 466b and 461b, 465b are examples of flow control devices that control flow of cannabis plant material to and from the decarboxylation device(s) 452b. Such flow control is provided in other ways in some embodiments, by controlling a speed of the conveyor(s) 464b for example. Examples of valves, conveyors and pipes, and other types of transfer mechanisms such as transfer mechanisms that include one or more vessels, are provided elsewhere herein. The valves in the transfer mechanisms 460b-1, 460b-2 carry different reference numbers to illustrate that different transfer mechanisms possibly include different types of valves.

In some embodiments, one or more solvents are added to a decarboxylation device 452b. For example, one or more extraction solvents added to a decarboxylation device 452b after decarboxylation could aid in transferring decarboxylated cannabis material to an extraction subsystem. In some embodiments, one or more solvents are added after decarboxylated cannabis material has been transferred from a decarboxylation device 452b to clean the decarboxylation device. In the decarboxylation subsystem 420b, the solvent(s) are added to the decarboxylation device(s) 452b from one or more solvent supplies 470b. A solvent supply 470b could be coupled to or otherwise in communication with one or more controllers 426b and/or one or more sensors 428b to control and/or monitor supply of solvent(s) to the decarboxylation device(s) 452b. Solvent transfer between the solvent supply(ies) 470b and the decarboxylation device(s) 452b, and/or other components of the decarboxylation subsystem 420b, could be through one or more transfer mechanism(s) that have not been shown in FIG. 4B to avoid congestion in the drawing. Such transfer mechanism(s) could be active or passive, and include one or more flow control devices such as valves in some embodiments.

In an embodiment, a vessel (or each vessel) 450b is weighed using the scale(s) 430b-1, to quantify inputs to the decarboxylation subsystem 420b. The cannabis material in the vessel(s) 450b is then transferred to the decarboxylation device(s) 452b, to heat the cannabis material as described elsewhere herein. One or more of the controller(s) 426b could be connected to or otherwise in communication with the decarboxylation device(s) 452b, to control the decarboxylation device(s). One or more sensor(s) 428b could similarly be connected to or otherwise in communication with the decarboxylation device(s) 452b, to measure one or more parameters and/or otherwise monitor one or more properties of a decarboxylation process or equipment. Temperature sensors to measure temperature of cannabis plant material during decarboxylation and weight sensors to measure weight of cannabis plant material during decarboxylation are examples.

Decarboxylated cannabis material is then transferred to the vessel(s) 456b. The decarboxylated cannabis material holding vessel(s) 456b are weighed by the scale(s) 430b-2. Weights as measured by the scale(s) 430b-2 could be used, for example, to reconcile input source product with total output extracted product, and/or otherwise to maintain desired and/or required records of cannabis material during processing.

An example extraction subsystem 420c is shown in FIG. 4C, and includes one or more computers 424c, one or more controllers 426c, one or more sensors 428c, and one or more scales at 430c-1 and/or 430c-2. These components are connected to or otherwise in communication with the server 418c. Implementation options for all of these components are described herein, at least above with reference to FIG. 4A. Although two sets of scale(s) are shown in at 430c-1, 430c-2, in some embodiments an extraction subsystem includes only one set of one or more scales.

The extraction subsystem 420c further includes one or more source material holding vessels 450c, one or more extractors 452c and one or more extract holding vessels 456c. The vessels 450c, 456c could include any of various types of container, and different container types could be used for source cannabis material and extract. The source cannabis material holding vessel(s) 450c could contain decarboxylated cannabis material, for example. In some embodiments, the vessel(s) 450c are or include the same vessel(s) as shown at 456a in FIG. 4A and/or 456b in FIG. 4B.

Transfer mechanism(s) 460c-1, 460c-2 are also shown in FIG. 4C. As examples, the transfer mechanism 460c-1 is shown as including valve(s) 461c, 465c and one or more pipes 463c, and the transfer mechanism 460c-2 is shown as including pump(s) 467c, 469c and one or more pipes 463c. In some embodiments, a transfer mechanism includes both valves and pumps. The valve(s) 461c, 465c and the pump(s) 467c, 469c are examples of flow control devices that control flow of cannabis plant material to and from the extractor(s) 452c. Examples of valves, pumps and pipes, and other types of transfer mechanisms, are provided elsewhere herein.

In some embodiments, one or more solvents are added to an extractor 452c. One or more extraction solvents could also or instead be added to cannabis material upstream of the extractor(s) 452c. For example, one or more extraction solvents added during or after milling and/or decarboxylation to aid in transferring cannabis material between processing stations or subsystems. In the example shown in FIG. 4C, one or more solvent(s) are added to the extractor(s) 452c from one or more solvent supplies 470c. A solvent supply 470c could be coupled to or otherwise in communication with one or more controllers 426c and/or one or more sensors 428c to control and/or monitor supply of solvent(s) to the extractor(s) 452c. Solvent transfer between the solvent supply(ies) 470c and the extractor(s) 452c, and/or other components of the extraction subsystem 420c, could be through one or more transfer mechanism(s) that have not been shown in FIG. 4C to avoid congestion in the drawing. Such transfer mechanism(s) could be active or passive, and include one or more flow control devices such as valves in some embodiments.

Extraction solvent need not be supplied in all embodiments. For example, in some embodiments non-solvent extraction such as mechanical extraction is implemented at 452c.

In an embodiment, a vessel (or each vessel) 450c is weighed using the scale(s) 430c-1, to quantify inputs to the extraction subsystem 420c. The cannabis material in the vessel(s) 450c is then transferred to the extractor(s) 452c, which implements any of various extraction processes to produce one or more extracts from the input cannabis material. Examples of extraction processes and extracts are disclosed elsewhere herein.

One or more of the controller(s) 426c could be connected to or otherwise in communication with the extractor(s) 452c, to control the extractor(s). One or more sensor(s) 428c could similarly be connected to or otherwise in communication with the extractor(s) 452c, to measure one or more parameters and/or otherwise monitor one or more properties of an extraction process or equipment.

The produced extract(s) are transferred to one or more vessels 456c. The vessel(s) 456c are weighed by the scale(s) 430c-2. Weights as measured by the scale(s) 430c-2 could be used, for example, to reconcile input cannabis material with total output extract(s), and/or otherwise to maintain desired and/or required records of cannabis material during processing.

An extract is further processed in some embodiments, through winterization and/or distillation for example.

FIG. 4D illustrates an example winterization subsystem 420d, which includes one or more computers 424d, one or more controllers 426d, one or more sensors 428d, and one or more scales at 430d-1 and/or 430d-2. These components are connected to or otherwise in communication with the server 418d. Implementation options for all of these components are described herein, at least above with reference to FIG. 4A. Although two sets of scale(s) are shown in at 430d-1, 430d-2, in some embodiments a winterization subsystem includes only one set of one or more scales.

The winterization subsystem 420d further includes one or more source material holding vessels 450d, one or more winterization chillers 452d, and one or more winterized cannabis material holding vessels 456d. The vessels 450d, 456d could include any of various types of container, and different container types could be used for input cannabis material at 450d and output cannabis material at 456d. The vessel(s) 450d hold cannabis extract-based material in some embodiments, and could be or include one or more of the vessel(s) 456c in FIG. 4C for example.

Transfer mechanism(s) 460d-1, 460d-2 are also shown in FIG. 4D. As an example, the transfer mechanisms 460d-1, 460d-2 are shown as including pump(s) 467d, 469d and one or more pipes 463d. In some embodiments, a transfer mechanism includes valves instead of or in addition to pumps. The pump(s) 467d, 469d are examples of flow control devices that control flow of cannabis plant material to and from the winterization chiller(s) 452d. Examples of valves, pumps and pipes, and other types of transfer mechanisms, are provided elsewhere herein.

In some embodiments, one or more solvents are added to a winterization chiller 452d. One or more winterization solvents could also or instead be added to cannabis material upstream of the winterization chiller(s) 452d. For example, one or more winterization solvents could be added during or after extraction to also aid in transferring cannabis material between an extraction subsystem and the winterization subsystem 420d. In the example shown in FIG. 4D, one or more solvent(s) are added to the winterization chiller(s) 452d from one or more solvent supplies 470d. A solvent supply 470d could be coupled to or otherwise in communication with one or more controllers 426d and/or one or more sensors 428d to control and/or monitor supply of solvent(s) to the winterization chiller(s) 452d.

Solvent recovery is provided at 471d in some embodiments. As noted elsewhere herein, solvent(s) may be recovered and reused. Solvent(s) recovered at 471d are returned to the solvent(s) supply(ies) 470d in the example shown, although recovered solvent(s) could also or instead be provided directly to the winterization chiller(s) 452d for reuse. A distillation apparatus is one example of a solvent recovery device or system that is implemented at 471d in some embodiments. Examples of distillation apparatus that can be used at 471d includes a rotary evaporator or a falling film evaporator (such as the AutoVap™ from TruSteel, Grass Valley, USA).

Solvent transfer between the solvent supply(ies) 470d and the winterization chiller(s) 452d, and/or other components of the winterization subsystem 420d, could be through one or more transfer mechanism(s) that have not been shown in FIG. 4D to avoid congestion in the drawing. Such transfer mechanism(s) could be active or passive, and include one or more flow control devices such as valves in some embodiments.

In an embodiment, a vessel (or each vessel) 450d is weighed using the scale(s) 430d-1, to quantify inputs to the winterization subsystem 420d. The cannabis material in the vessel(s) 450d is then transferred to the winterization chiller(s) 452d. An example of a winterization chiller 452d is a refrigerator, and other examples are disclosed herein. In some embodiments, the winterization chiller(s) 452d are provided to cool a mixture of extract and winterization/polar solvent(s) to a temperature at which waxes and/or lipids separate from the extract. The winterization chiller(s) 452d also include one or more devices or elements to remove one or more undesirable components from a crude cannabis extract. Examples are disclosed elsewhere herein. These devices or elements are not separately shown in FIG. 4D to avoid further congestion in the drawing.

One or more of the controller(s) 426d could be connected to or otherwise in communication with the winterization chiller(s) 452d, to control the winterization chiller(s). One or more sensor(s) 428d could similarly be connected to or otherwise in communication with the winterization chiller(s) 452d, to measure one or more parameters and/or otherwise monitor one or more properties of a winterization process or equipment.

One or more outputs of the winterization chiller(s) 452d are transferred to one or more vessel(s) 456d. The vessel(s) 456d are weighed by the scale(s) 430d-2. Weights as measured by the scale(s) 430d-2 could be used, for example, to reconcile input cannabis material with total output winterized extract(s), and/or otherwise to maintain desired and/or required records of cannabis material during processing.

Another purification process that is provided in some embodiments is distillation. FIG. 4E illustrates an example distillation subsystem 420e, which includes one or more computers 424e, one or more controllers 426e, one or more sensors 428e, and one or more scales at 430e-1 and/or 430e-2. These components are connected to or otherwise in communication with the server 418e. Implementation options for all of these components are described herein, at least above with reference to FIG. 4A. Although two sets of scale(s) are shown in at 430e-1, 430e-2, in some embodiments a distillation subsystem includes only one set of one or more scales.

The distillation subsystem 420e further includes one or more source material holding vessels 450e, one or more distillers 452e, and one or more distillate holding vessels 456e. The vessels 450e, 456e could include any of various types of container, and different container types could be used for input cannabis material at 450e and output cannabis distillate at 456e. The vessel(s) 450e hold cannabis extract-based material, which has been winterized in some embodiments, and could be or include one or more of the vessel(s) 456c in FIG. 4C and/or 456d in FIG. 4D for example.

Transfer mechanism(s) 460e-1, 460e-2 are also shown in FIG. 4E. As an example, the transfer mechanisms 460e-1, 460e-2 are shown as including pump(s) 467e, 469e and one or more pipes 463e. In some embodiments, a transfer mechanism includes valves instead of or in addition to pumps. The pump(s) 467e, 469e are examples of flow control devices that control flow of cannabis plant material to and from the distiller(s) 452e. Examples of valves, pumps and pipes, and other types of transfer mechanisms, are provided elsewhere herein.

In some embodiments, one or more solvents are added to a distiller 452e. One or more solvents could also or instead be added to cannabis material upstream of the distiller(s) 452e. For example, one or more solvents could be added during or after extraction and/or winterization to also aid in transferring cannabis material from an extraction subsystem and/or a winterization subsystem. In the example shown in FIG. 4E, one or more solvent(s) are added to the distiller(s) 452e from one or more solvent supplies 470e. A solvent supply 470e could be coupled to or otherwise in communication with one or more controllers 426e and/or one or more sensors 428e to control and/or monitor supply of solvent(s) to the distiller(s) 452e. Solvent recovery is provided at 471e in some embodiments, and examples of solvent recovery are provided elsewhere herein. Solvent(s) are recovered during distillation and are collected as one or more outputs of the distiller(s) 452e in some embodiments. Solvent(s) recovered at 471e are returned to the solvent(s) supply(ies) 470e in the example shown, although recovered solvent(s) could also or instead be provided directly to the distiller(s) 452d for reuse.

Solvent transfer between the solvent supply(ies) 470e and the distiller(s) 452e, and/or other components of the distillation subsystem 420e, could be through one or more transfer mechanism(s) that have not been shown in FIG. 4E to avoid congestion in the drawing. Such transfer mechanism(s) could be active or passive, and include one or more flow control devices such as valves in some embodiments.

In an embodiment, a vessel (or each vessel) 450e is weighed using the scale(s) 430e-1, to quantify inputs to the distillation subsystem 420e. The cannabis material in the vessel(s) 450e is then transferred to the distiller(s) 452e. An example of a distiller 452e is a distillation column, to separate one or more cannabinoids and/or terpenes from extract(s).

One or more of the controller(s) 426e could be connected to or otherwise in communication with the distiller(s) 452e, to control the distiller(s). The sensor(s) 428e could similarly be connected to or otherwise in communication with the distiller(s) 452e, to measure one or more parameters and/or otherwise monitor one or more properties of a distillation process or equipment.

One or more outputs of the distiller(s) 452e are transferred to the vessel(s) 456e. The vessel(s) 456e are weighed by the scale(s) 430e-2. Weights as measured by the scale(s) 430e-2 could be used, for example, to reconcile input cannabis material with total output distillate, and/or otherwise to maintain desired and/or required records of cannabis material during processing.

The example system 400 shown in FIGS. 4A-4E and described in detail herein represents one illustrative embodiment. Other embodiments are also possible. For example, although various components are shown separately in these drawings, multiple components could be implemented in a single component. In some embodiments, any two or more of the computer(s), controller(s), sensor(s), and scale(s) in a subsystem, or possibly such components of multiple subsystems, are implemented using a single device.

In other embodiments, other types of subsystems are also or instead provided. For example, one or more pre-treatment subsystems are implemented in a similar manner as other subsystems in some embodiments. One embodiment of a pre-treatment subsystem is the drying system disclosed in Canadian Patent Application No. 3,033,404, filed on Feb. 11, 2019. Other pre-treatment subsystems are implemented in substantially the same way in some embodiments, with different sets of one or more pre-treatment devices. Referring to FIG. 4B as an example, in some embodiments a pre-treatment subsystem includes the same components, with the exception that one or more pre-treatment devices are provided at 452b instead of a device to perform decarboxylation. The solvent(s) supply(ies) 470b might not necessarily be provided in a pre-treatment subsystem, depending on the pre-treatment(s) to be performed. The numbers and/or type(s) of other components, such as vessel(s), transfer mechanism(s), scale(s), controller(s), and/or sensor(s), in a pre-treatment subsystem may also vary depending on the pre-treatment(s) to be performed.

In general, in some embodiments of a pre-treatment subsystem one or more computers, one or more controllers, one or more sensors, and one or more sets of scales are connected to or otherwise in communication with a server, and implementation options for all of these components are described elsewhere herein. One or more source material holding vessels are coupled to one or more pre-treatment devices through one or more transfer mechanisms, and the one or more pre- treatment devices are coupled to one or more pre-treated material holding vessels through one or more transfer mechanisms. Examples of vessels and transfer mechanisms are also provided elsewhere herein.

For solvent-based embodiments, one or more solvents are added to a vessel and/or a pre-treatment device, from one or more solvent supplies, for example. Solvent(s) are also recovered and reused in some embodiments, and examples of solvent recovery options are provided elsewhere herein. Pre-treatment could, but need not necessarily, involve a solvent.

One or more controllers in a pre-treatment subsystem are connected to or otherwise in communication with the pre-treatment device(s), to control the pre-treatment device(s). One or more sensors are connected to or otherwise in communication with the pre-treatment device(s), to measure one or more parameters and/or otherwise monitor one or more properties of a pre-treatment process or equipment.

One or more source material vessels and/or one or more pre-treated material vessels are weighed by one or more scales in some embodiments. Weights as measured by the scale(s) could be used, for example, to reconcile input cannabis material with total output cannabis material, and/or otherwise to maintain desired and/or required records of cannabis material during processing.

As noted herein, distillation is one example of a purification process. Some embodiments also or instead include a separation subsystem, to purify a cannabis extract or further purify a distilled cannabis extract for example. A separation subsystem is implemented in substantially the same way as the distillation subsystem 420e in FIG. 4E in some embodiments, with the exception that one or more separators, such as membrane filtration or separation systems, are provided at 452e instead of the distiller(s) 452e. The solvent(s) supply(ies) 470e might not necessarily be provided in a separation subsystem, depending on the type(s) of separation to be performed. The numbers and/or type(s) of other components, such as vessel(s), transfer mechanism(s), scale(s), controller(s), and/or sensor(s), in a separation subsystem may also vary depending on the type(s) of separation to be performed.

In general, in some embodiments of a separation subsystem one or more computers, one or more controllers, one or more sensors, and one or more sets of scales are connected to or otherwise in communication with a server, and implementation options for all of these components are described elsewhere herein. One or more source material holding vessels are coupled to one or more separators through one or more transfer mechanisms, and the one or more separators are coupled to one or more separated material holding vessels through one or more transfer mechanisms. Examples of vessels and transfer mechanisms are also provided elsewhere herein.

For solvent-based embodiments, one or more solvents are added to a vessel and/or a separator, from one or more solvent supplies, for example. Solvent(s) are also recovered and reused in some embodiments, and examples of solvent recovery options are provided elsewhere herein. Separation could, but need not necessarily, involve a solvent. A membrane filtration or separation system is an example of an separator to separate one or more cannabinoids and/or terpenes, from extract(s) and/or distillate(s) for example.

One or more controllers are connected to or otherwise in communication with the separator(s), to control the separator(s). One or more sensor(s) are connected to or otherwise in communication with the separator(s) in some embodiments, to measure one or more parameters and/or otherwise monitor one or more properties of a separation process or equipment.

One or more source material vessels and/or one or more separated material vessels are weighed by one or more scales in some embodiments. Weights as measured by the scale(s) could be used, for example, to reconcile input cannabis material with total output cannabis material, and/or otherwise to maintain desired and/or required records of cannabis material during processing.

Pre-treatment subsystems and separation subsystems are examples of other subsystems that are not explicitly shown in the system 40 but are provided in some embodiments. Other subsystems are also possible.

A system such as the system 400 enables either or both of distributed control and centralized control. Referring again to FIGS. 4A and 4B, for example, in some embodiments the controller(s) 426a, 426b provide local control of at least some components of the milling subsystem 420a and the decarboxylation subsystem 420b, respectively. Such local, per-subsystem or intra-subsystem control is an example of distributed control. Centralized control is also possible, with at least some control functions centralized and/or coordinated at a server 402, 418a, and/or 418b, for example. Local controllers 426a, 426b could still be provided to actually carry out control actions in each subsystem, but at least some aspects of system control and operation involve at least information that is associated with multiple subsystems. For example, in some embodiments a server-based central controller monitors processing system operating conditions, determines control actions based on those conditions, and controls system components either directly or through local subsystem controllers such as 426a, 426b.

In a milling subsystem such as 420a, milling of cannabis plant material by the milling machine(s) 452a is likely a processing bottleneck. Transfer of cannabis plant material to and from the milling machine(s) 452a by the transfer mechanism(s) 460a-1, 460a-2 does not involve actually processing that plant material, and therefore physically moving the plant material by means of the transfer mechanisms is not expected to introduce significant delay. Transfer mechanism control, however, can still be an important part of overall processing system control. For example, in some embodiments monitoring of the milling machine(s) 452a and controlling the transfer mechanisms 460a-1, 460a-2 based on such monitoring helps reduce or avoid backup of excess input material to the milling machine(s), shortage of input material to the milling machine(s), backup of output milled material from the milling machine(s), and/or shortage output milled material from the milling machine(s).

In an embodiment, measurements by the scale(s) 430a-1, 430a-2 are used to calculate or otherwise determine rates at which plant material is currently being input to and output from the milling machine(s) 452. A mismatch between these rates is used in some embodiments to determine an adjustment to a flow rate through one or both of the transfer mechanisms 460a-1, 460a-2, that should at least reduce the mismatch and bring input and output flow rates closer to a match. Flow rate adjustments are made in the milling subsystem 420a by controlling one or more valves such as 462a, 466a and/or speed of one or more conveyors such as 464a for example. An inability to reduce a rate mismatch, after a certain number of adjustment and/or monitoring cycles for example, is treated as an error condition in some embodiments and is indicative of such undesirable operating conditions such as spillage of material at milling machine input(s) and/or output(s), full or partial blockage of a processing path or line, and/or malfunction of one or more milling machine(s) 452a.

For control based on weight monitoring, the scale(s) 430a-1, 430a-2 collect weight measurements. In some embodiments, the weight measurements are provided by the scale(s) 430a-1, 430a-2 to the server 418a, and the server provides the weight measurements to one or more local controllers 426a or to a central controller at the server itself and/or at the server 402. The controller(s) receive the weight measurements, and then determine any adjustments and/or error conditions, as well as appropriate control actions based on the weight measurements. Control actions are then implemented or performed by controlling one or more subsystem components. Examples of control actions associated with the milling subsystem 420a include increasing or decreasing an input material transfer rate, increasing or decreasing an output material transfer rate, increasing or decreasing milling machine speed, and distributing material to more or fewer milling machines by bringing more milling machines online or shutting down one or more milling machines that are currently online for example.

It should be noted that processing control need not necessarily always be geared toward increasing processing speed. Under certain conditions, such as a downstream bottleneck in a processing system, it is possible that processing speed should be reduced. In that event, one or more system components are controlled accordingly, to reduce a rate of transfer of cannabis material through at least part of a processing system.

Rate mismatch is one example of a control parameter or condition based upon which one or more system components are controlled. Control is also or instead based on any of various other conditions in further embodiments, and additional examples are disclosed herein.

Even in the milling subsystem 420a, other sensors 428a are also or instead used in some embodiments, to measure or otherwise determine and report operating conditions or parameters that are taken into account in processing system control. Examples of sensors 428a include valve position sensors, conveyor speed sensors, milling machine motor speed sensors, milling machine temperature sensors, and milling machine power consumption sensors. Based on weight measurements from the scale(s) 430a-2 and a speed measurement from a sensor 428a that measures speed of a conveyor of a transfer mechanism 460a-2 for example, a controller 426a and/or a server-based controller at the server 418a and/or the server 402 is able to determine whether a conveyor speed increase (or decrease) had an intended effect on actual flow rate of milled cannabis material from the milling machine(s) 452a. If not, then this indicates to the controller(s) that further adjustment of flow rate should target one or more other system components rather than the conveyor in a transfer mechanism 460a-2 in this example.

In some embodiment, one milling machine 452a is operated continuously to process a continuous stream of cannabis plant material and provide a continuous stream of milled cannabis plant material. In other embodiments, operation of multiple milling machines 452a is staged so that at least one milling machine is always available to receive input cannabis material and/or at least one milling machine is always providing output milled cannabis plant material. Such staged operation is controlled based on any of one or more sensor readings indicative of milling progress at the milling machines 452a in some embodiments. Each individual milling machine might operate in a batch or non-continuous mode, but overall continuous processing can still be provided.

One or more solvents are used in some embodiments, for example to aid in cleaning the milling machine(s) 452a and/or to aid in transferring reduced size cannabis plant material from the milling machine(s). In FIG. 4A, any such solvent(s) are added from the solvent supply(ies) 470a. One or more transfer mechanism(s) are provided in some embodiments to control flow of solvent(s) from the solvent supply(ies) 470a to the milling machine(s) 452a and/or other subsystem components. For example, in an embodiment the sensor(s) 428a include one or more flow sensors to measure solvent flow from one or more of the solvent supply(ies) 470a, and one or more one or more transfer mechanism components such as valves and/or pumps coupled to the solvent supply(ies) are controllable to control a flow rate and/or amount of any solvent(s) dispensed from the solvent supply(ies). In embodiments, solvent dispensing from the solvent supply(ies) 470a is controlled based on any one or more of: an amount of cannabis plant material milled by a milling machine 452a; a rate of milling; an input flow rate of cannabis plant material into a milling machine; an output flow rate of milled cannabis plant material out of a milling machine; type(s) of cannabis plant material milled in a milling machine; whether solvent is to be added before, during, or after milling; an amount of time since a milling machine was previously cleaned; and an amount of milling residue detected in a milling machine by a sensor 428a.

Turning to the decarboxylation subsystem 420b in FIG. 4B, in some embodiments measurements from one or more scale(s) 430b-1, 430b-2 and/or sensors 428b, and/or possibly other information about the subsystem, the type(s) of cannabis material being processed, and/or the decarboxylation process(es) to be performed are taken into account for the purpose of system control. For example, flow control as described with reference to FIG. 4A is an example of system control that could also or instead be implemented in the decarboxylation subsystem 420b based on measurements from one or more scale(s) 430b-1, 430b-2 and/or sensors 428b. Although only one control connection from the controller(s) 426b to the transfer mechanism(s) 460b-2 and only one connection from the sensor(s) 428b to each of the transfer mechanism(s) 460b-1, 460b-2 are shown in FIG. 4B, this is solely to avoid congestion in the drawings. Any of various types of controller(s) and/or sensor(s) could be provided to control and/or monitor multiple system conditions and/or operating conditions associated with the transfer mechanism(s) 460b-1, 460b-2.

In the example decarboxylation subsystem 420b, control of material flow through the transfer mechanism(s) 460b-1 is by adjustment of one or both of the valve(s) 462b, 466b and/or speed of the conveyor 464b. For the transfer mechanism(s) 460b-2, the valve(s) 461b, 465b are controllable to control material flow through the pipe(s) 463b. In other embodiments, similar, different, and/or additional components are controllable to adjust material flow during processing. For example, in a flow control embodiment, input flow rate(s) to the decarboxylation device(s) 452b and/or output flow rate(s) from the decarboxylation device(s) are controlled to match or be within a certain range of decarboxylation rate(s).

Regarding the decarboxylation device(s) 452b, as noted elsewhere herein decarboxylation of cannabinoid acids is a function of time and temperature in some embodiments. Time and temperature are therefore examples of control parameters for the decarboxylation device(s) 452b. In some embodiments, temperature of cannabis material in a decarboxylation device 452b is measured by a sensor 428b such as a temperature probe or thermometer, and a heater in the decarboxylation device is controlled based on one or more temperature measurements, and possibly other conditions or parameters such as reducing or minimizing thermal degradation of desirable pharmacological cannabinoids into undesirable degradation products. Weight of cannabis plant material is also or instead monitored in other embodiments and used in controlling the decarboxylation device 452b and/or other components.

In some embodiments, control of material transfer and decarboxylation is coordinated. For example, cannabis plant material might not be transferred to a decarboxylation device 452b until a temperature probe or thermometer for internal heater temperature measures a minimum temperature, such as 120° C., at which time the transfer mechanism(s) 460b-1 is controlled to transfer cannabis plant material from the vessel(s) 450b to the decarboxylation device. Multiple decarboxylation devices 452b and/or one or more decarboxylation device(s) that are monitored and controlled to maintain a target decarboxylation temperature are used in some embodiments to enable substantially continuous transfer of cannabis plant material for decarboxylation and substantially continuous decarboxylation. For example, operation of multiple decarboxylation devices 452b is staged in some embodiments so that at least one decarboxylation device is always available to receive input cannabis material and/or at least one decarboxylation device is always providing output decarboxylated cannabis material. Such staged operation is controlled based on any of one or more sensor readings indicative of decarboxylation progress at the decarboxylation devices in some embodiments.

One or more solvents are used in some embodiments, for example to aid in cleaning the decarboxylation device(s) 452b and/or to aid in transferring decarboxylated cannabis plant material from the decarboxylation device(s). In FIG. 4B, any such solvent(s) are added from the solvent supply(ies) 470b. One or more transfer mechanism(s) are provided in some embodiments to control flow of solvent(s) from the solvent supply(ies) 470b to the decarboxylation device(s) 452b and/or other subsystem components. For example, in an embodiment the sensor(s) 428b include one or more flow sensors to measure solvent flow from one or more of the solvent supply(ies) 470b, and one or more transfer mechanism components such as valves and/or pumps coupled to the solvent supply(ies) are controllable to control a flow rate and/or amount of any solvent(s) dispensed from the solvent supply(ies). In embodiments, solvent dispensing from the solvent supply(ies) 470b is controlled based on any one or more of: an amount of cannabis plant material decarboxylated by a decarboxylation device 452b; a rate of decarboxylation; an input flow rate of cannabis plant material into a decarboxylation device; an output flow rate of cannabis plant material out of a decarboxylation device; type(s) of cannabis plant material decarboxylated in a decarboxylation device; whether solvent is to be added before, during, or after decarboxylation; an amount of time since a decarboxylation devicewas previously cleaned; and an amount of cannabis plant residue detected in a decarboxylation device by a sensor 428b.

In the extraction subsystem 420c in FIG. 4C, cannabis material flow rate(s) into and/or out of the extractor(s) 452c are controllable in some embodiments in much the same manner as other flow rate examples discussed herein. For example, in some embodiments measurements from one or more scale(s) 430c-1, 430c-2 and/or sensors 428c, and/or possibly other information about the subsystem, the type(s) of cannabis material being processed, and/or the extraction process(es) to be performed are taken into account for the purposes of system control. Flow control as described with reference to FIG. 4A and/or FIG. 4B is an example of system control that could also or instead be implemented in the extraction subsystem 420c based on measurements from one or more scale(s) 430c-1, 430c-2 and/or sensors 428c. Although only one control connection from the controller(s) 426c to the transfer mechanism(s) 460c-2 and only one connection from the sensor(s) 428c to each of the transfer mechanism(s) 460c-1, 460c-2 are shown in FIG. 4C, this is solely to avoid congestion in the drawings. Any of various types of controller(s) and/or sensor(s) could be provided to control and/or monitor multiple system conditions and/or operating conditions associated with the transfer mechanism(s) 460c-1, 460c-2.

In the example extraction subsystem 420c, control of material flow through the transfer mechanism(s) 460c-1 is by adjustment of one or both of the valve(s) 461c, 465c. For the transfer mechanism(s) 460c-2, the pump(s) 467c, 469c are controllable to control a rate of material flow through the pipe(s) 463c. In other embodiments, similar, different, and/or additional components are controllable to adjust material flow during processing. For example, in a flow control embodiment, input flow rate(s) to the extractor(s) 452c and/or output flow rate(s) from the extractor(s) are controlled to match or be within a certain range of extraction rate(s) in one embodiment.

Regarding the extractor(s) 452c, as noted elsewhere herein extraction involves an extraction solvent in some embodiments, and features provided by an extractor in some embodiments include pressure control, temperature control, extraction fluid flow rate control and/or control of other parameters of an extraction process. These are illustrative of parameters or conditions for which one or more components such as the extractor(s) 452c are controlled based on measurements from one or more scales(s) 430c-1, 430c-2 and/or one or more sensors 428c in some embodiments.

Consider supercritical fluid extraction with CO₂ or extraction with water as an example. In an embodiment, a transfer mechanism 460c-1 is controlled to transfer an amount of cannabis material into an extraction chamber of an extractor 452c, and the extractor is controlled to seal the extraction chamber, and the extraction chamber is then allowed to fill up, with CO₂ for CO₂ extraction or water for water extraction, by controlling inlet and outlet regulating valves on the extractor for example. The sensor(s) 428c include one or more CO₂ or fluid/water level or volume monitors in an embodiment, to monitor the amount of CO₂ or fluid/water in the extraction chamber(s) of the extractor(s) 452c. After an extraction chamber is filled to a target CO₂ or fluid/water level or concentration and has reached a stable pressure, as monitored by one or more pressure sensor(s) at 428c, a heater of the extractor is controlled to start heating the chamber. In some embodiments, the extraction chamber is left for a predefined time, such as 30 minutes, to allow the chamber to reach a stable temperature, as monitored by one or more temperature sensors at 428c for example. In some embodiments, temperature of cannabis material in an extractor 452c is also or instead measured by a sensor 428c such as a temperature probe or thermometer, and a heater in the extractor is controlled based on one or more cannabis material temperature measurements, and possibly other conditions or parameters.

After an extraction run is complete, in some embodiments the extraction chamber is purged with CO₂, by controlling inlet and outlet regulating valves on the extractor for example, to collect the produced cannabis extract, and the cannabis extract is transferred from the extractor(s) 452c by controlling one or more components of the transfer mechanism(s) 460c-2, such as one or more pumps 467c, 469c.

Even though the extraction process in this example is a batch process in which an amount of cannabis material is sealed inside an extraction chamber during extraction, multiple extractors 452c and/or one or more extractor(s) that implement a continuous extraction are used in some embodiments to enable substantially continuous transfer of cannabis material for extraction and substantially continuous extraction. For example, operation of multiple extractors 452c is staged in some embodiments so that at least one extractor is always available to receive input cannabis material and/or at least one extractor is always providing output cannabis extract. Such staged operation is controlled based on any of one or more sensor readings indicative of extraction progress at the extractors in some embodiments.

One or more extraction solvents are used in some embodiments. In FIG. 4C, the solvent(s) are added from the solvent supply(ies) 470c. One or more transfer mechanism(s) are provided in some embodiments to control flow of solvent(s) from the solvent supply(ies) 470c to the extractor(s) 452c, and/or possibly to other subsystem components. For example, in an embodiment the sensor(s) 428c include one or more flow sensors to measure solvent flow from one or more of the solvent supply(ies) 470c, and one or more transfer mechanism components such as valves and/or pumps coupled to the solvent supply(ies) are controllable to control a flow rate and/or amount of any solvent(s) dispensed from the solvent supply(ies). In embodiments, solvent dispensing from the solvent supply(ies) 470c is controlled based on any one or more of: an amount of cannabis plant material processed by an extractor 452c; an amount of solvent (if any) added to cannabis material upstream of an extractor; a rate of extraction; an input flow rate of cannabis plant material into an extractor; an output flow rate of cannabis extract out of an extractor; type(s) of cannabis plant material processed by an extractor; whether solvent is to be added before, during, or after extraction; an amount of time since an extractor was previously cleaned; and an amount of cannabis plant residue detected in an extractor by a sensor 428c.

Turning now to winterization and the example winterization subsystem 420d in FIG. 4D, cannabis material flow rate(s) into and/or out of the winterization chiller(s) 452d are controllable in some embodiments in much the same manner as other flow rate examples discussed herein. For example, in some embodiments measurements from one or more scale(s) 430d-1, 430d-2 and/or sensors 428d, and/or possibly other information about the subsystem, the type(s) of cannabis material being processed, and/or the winterization process(es) to be performed are taken into account for the purposes of system control. Flow control as described with reference to one or more of FIGS. 4A to 4C is an example of system control that could also or instead be implemented in the winterization subsystem 420d based on measurements from one or more scale(s) 430d-1, 430d-2 and/or sensors 428d. Although only one control connection from the controller(s) 426d to the transfer mechanism(s) 460d-2 and only one connection from the sensor(s) 428d to each of the transfer mechanism(s) 460d-1, 460d-2 are shown in FIG. 4D, this is solely to avoid congestion in the drawings. Any of various types of controller(s) and/or sensor(s) could be provided to control and/or monitor multiple system conditions and/or operating conditions associated with the transfer mechanism(s) 460d-1, 460d-2.

In the example extraction subsystem 420d, control of material flow through either or both of the transfer mechanism(s) 460d-1, 460d-2 is by adjustment of one or more pump(s) 467d, 469d. In other embodiments, similar, different, and/or additional components are controllable to adjust material flow during processing. For example, in a flow control embodiment, input flow rate(s) to the winterization chiller(s) 452d and/or output flow rate(s) from the extractor(s) are controlled to match or be within a certain range of extraction rate(s) in one embodiment.

Regarding the winterization chiller(s) 452d, as noted elsewhere herein winterization involves reducing temperature of a cannabis extract, which is mixed with a winterization solvent in some embodiments, to induce precipitation of undesirable components and thereby purify the extract. Flow rate through the winterization chiller(s) 452d, temperature in the winterization chiller(s) or parts thereof, and/or flow rate of winterization solvent are illustrative of parameters or conditions for which one or more components of the winterization chiller(s) 452d and/or other components of the winterization subsystem 420d are controlled based on measurements from one or more scales(s) 430d-1, 430d-2 and/or one or more sensors 428d in some embodiments.

According to illustrative examples provided above, removing waxy ballast from cannabis extract includes chilling a mixture of cannabis extract and winterization solvent to a temperature less than or equal to about 0° C., alternatively less than or equal to about −10° C., alternatively less than or equal to about −20° C., for a time period of at least 1 hour, alternatively at least about 24 hours, alternatively at least about 48 hours, alternatively at least about 50 hours, alternatively at least about 72 hours. Temperature at one or more locations or devices in the winterization chiller(s) 452d is controlled based on these temperature and/or time parameters and measurements by one or more of the sensor(s) 428d. Material flow rate(s) through one or more parts of the winterization chiller(s) 452d are also or instead controlled in some embodiments, by controlling one or more of pump(s) 467d, 469d in the transfer mechanism(s) 460d-1, 460d-2 and/or flow control components such as pumps and/or valves in the winterization chiller(s) 452d.

Temperature and/or flow rate control is based not only on target or setpoint parameters such as the above temperature and time parameters noted above, but also or instead on other information in some embodiments. Examples of such other information that is used in winterization temperature and/or flow rate control in some embodiments include any one or more of: measurements of extract temperature by one or more of the sensor(s) 428d such as one or more thermometers or temperature probes; measurements of extract flow rate by one or more of the sensor(s) 428d such as flow sensors; measurements of extract viscosity by one or more of the sensor(s) 428d such as viscosity sensors; measurements of extract density by one or more of the sensor(s) 428d such as density sensors; pressure measurements by one or more of the sensor(s) 428d such as pressure sensors, potentially indicating flow restriction with the winterization chiller(s) 452d; and measurements of precipitate buildup in the winterization chiller(s) or components therein such as filters, by one or more of the sensor(s) 428d.

Winterization is a continuous process in some embodiments, and input flow rate(s) and output flow rate(s) through the transfer mechanisms 460d-1, 460d-2, respectively, are controlled to match or substantially match a rate of flow of extract through the winterization chiller(s) 452d. In some embodiments, operation of multiple winterization chiller(s) 452d is staged so that at least one winterization chiller is always available to receive input cannabis material and/or at least one winterization chiller is always providing output winterized cannabis extract. Such staged operation is controlled based on any of one or more sensor readings indicative of winterization progress at the winterization chillers in some embodiments.

One or more winterization solvents are added from the solvent supply(ies) 470d in some embodiments, and one or more transfer mechanism(s) are provided in some embodiments to control flow of solvent(s) from the solvent supply(ies) to the winterization chiller(s) 452d, and/or possibly to other subsystem components. For example, in an embodiment the sensor(s) 428d include one or more flow sensors to measure solvent flow from one or more of the solvent supply(ies) 470d, and one or more transfer mechanism components such as valves and/or pumps coupled to the solvent supply(ies) are controllable to control a flow rate and/or amount of any solvent(s) dispensed from the solvent supply(ies). In embodiments, solvent dispensing from the solvent supply(ies) 470d is controlled based on any one or more of: an amount of cannabis extract processed by a winterization chiller 452d; a rate of winterization; an input flow rate of cannabis extract into a winterization chiller; an output flow rate of cannabis extract out of a winterization chiller; type(s) of cannabis extract processed by a winterization chiller; and whether solvent is to be added before or during winterization. In some embodiments, one or more solvents are also or instead used to aid in cleaning a winterization chiller 452d, and in such embodiments solvent dispensing from the solvent supply(ies) 470d could be controlled based on any one or more of: an amount of time since an extractor was previously cleaned; and an amount of precipitate residue detected in an extractor by a sensor 428d, for example.

As in other subsystems, cannabis material flow rate(s) into and/or out of the distiller(s) 452e in the distillation subsystem 420e in FIG. 4E are controllable in some embodiments. For example, in some embodiments measurements from one or more scale(s) 430e-1, 430e-2 and/or sensors 428e, and/or possibly other information about the subsystem, the type(s) of cannabis material being processed, and/or the distillation process(es) to be performed are taken into account for the purposes of system control. Flow control as described with reference to one or more of FIGS. 4A to 4D is an example of system control that could also or instead be implemented in the distillation subsystem 420e based on measurements from one or more scale(s) 430e-1, 430e-2 and/or sensors 428e. Although only one control connection from the controller(s) 426e to the transfer mechanism(s) 460e-2 and only one connection from the sensor(s) 428e to each of the transfer mechanism(s) 460e-1, 460e-2 are shown in FIG. 4E, this is solely to avoid congestion in the drawings. Any of various types of controller(s) and/or sensor(s) could be provided to control and/or monitor multiple system conditions and/or operating conditions associated with the transfer mechanism(s) 460e-1, 460e-2.

In the example distillation subsystem 420e, the pump(s) 467e, 469e are controllable to control a rate of material flow through the pipe(s) 463e in the transfer mechanism(s) 460e-1, 460e-2. In other embodiments, similar, different, and/or additional components are controllable to adjust material flow during processing. For example, in a flow control embodiment, input flow rate(s) to the distiller(s) 452e and/or output flow rate(s) from the distiller(s) are controlled to match or be within a certain range of distillation rate(s) in one embodiment.

Regarding the distiller(s) 452e, as noted elsewhere herein distillation involves purifying, isolating and/or crystallizing at least one cannabinoid from a cannabis extract. Features provided by a distiller in some embodiments include pressure control, temperature control, cannabis extract flow rate control, vapor flow control and/or control of other parameters of a distillation process. These are illustrative of parameters or conditions for which one or more components such as the distiller(s) 452e are controlled based on measurements from one or more scales(s) 430e-1, 430e-2 and/or one or more sensors 428e in some embodiments.

According to examples described above, a distiller 452e includes one or more flasks, heating elements, pumps, and cooling channels, and in some embodiments cannabis extract that is received for distillation is held in an input flask of a distiller and heated to evaporate at least a portion of the extract. A rate of flow of cannabis extract into a distiller 452e is controlled by controlling a transfer mechanism 460e-1 or such components as one or more of the pump(s) 467e, 469e in the example shown in FIG. 4E. The flow rate control is based on such parameters as capacity of the input flask, fill level of the input flask as measured by a sensor 428e such as a level sensor, and a rate of distillation as measured by a sensor 428e such as a vapor sensor in some embodiments. Vaporized cannabinoids and terpenes flow into one or more cooling channels in the distiller(s) 452e, and in some embodiments flow control components such as vacuum pumps in the distiller(s) are controlled based on vapor detection or measurement by one or more vapor sensors at 428e.

Cannabinoids and terpenes that condense at different points in cooling channels of the distiller(s) based on their respective condensation temperatures are separated into different collection flasks or containers. Flow rate through the distiller(s) 452e and temperature in the distiller(s) or parts thereof are illustrative of parameters or conditions for which one or more components of the distiller(s) 452e and/or other components of the distillation subsystem 420e are controlled based on any of various measurements in some embodiments. For example, in some embodiments vapor flow rate is increased (or decreased) in response to vapor temperature measurements, by one or more sensors 428e such as thermometers or temperature probes, that are below (or above) a target temperature at one or more particular locations in a distillation column. One or more heaters and/or coolers in the distiller(s) 452e are also or instead controlled in some embodiments to maintain target vapor temperature(s). These flow and temperature control examples are illustrative of control actions that could be taken to help control the location or position in a distiller 452e at which different components of vapor condense for collection.

In some embodiments, operation of multiple distiller(s) 452e is staged so that at least one distiller is always available to receive input cannabis extract and/or at least one distiller is always providing output distillate. Such staged operation is controlled based on any of one or more sensor readings indicative of distillation progress at the distillers in some embodiments.

One or more solvents are used in some embodiments, to aid in cleaning the distiller(s) 452e for example. In FIG. 4E, the solvent(s) are added from the solvent supply(ies) 470e. One or more transfer mechanism(s) are provided in some embodiments to control flow of solvent(s) from the solvent supply(ies) 470e to the distiller(s) 452e, and/or possibly to other subsystem components. For example, in an embodiment the sensor(s) 428e include one or more flow sensors to measure solvent flow from one or more of the solvent supply(ies) 470e, and one or more transfer mechanism components such as valves and/or pumps coupled to the solvent supply(ies) are controllable to control a flow rate and/or amount of any solvent(s) dispensed from the solvent supply(ies). In embodiments, solvent dispensing from the solvent supply(ies) 470e is controlled based on any one or more of: an amount of cannabis extract processed by a distiller 452e; a rate of distillation; an input flow rate of cannabis extract into a distiller; an output flow rate of cannabis distillate out of a distiller; type(s) of cannabis extract processed by a distiller; an amount of time since a distiller was previously cleaned; and an amount of residue detected in a distiller by a sensor 428e.

The examples above refer to the subsystems shown in FIGS. 4A to 4E, and are intended to illustrate how such subsystems are controlled in some embodiments. Devices or equipment within such subsystems, other than the devices or equipment explicitly referenced in the examples, are also or instead controlled based on one or more measured parameters and/or control parameters or setpoints in other embodiments. One or more mixture vessels and one or more centrifuges in a winterization chiller are examples of such other devices or equipment.

In some embodiments other subsystems, such as pre-treatment subsystems and/or separation subsystems, are also or instead controlled based on one or more measured parameters and/or control parameters or setpoints

These control examples for the subsystems shown in FIGS. 4A to 4E are illustrative of per-subsystem control embodiments. In some embodiments, coordinated inter-subsystem control is also possible.

For example, in some embodiments a solvent that is added to aid in cleaning a milling machine, cleaning a decarboxylation device, and/or moving cannabis material between processing stations or subsystems is also effective as an extraction solvent. In such embodiments, a possible application of coordinated control is in optimizing or at least improving solvent usage efficiency.

Consider an embodiment in which an extraction solvent is added to a milling machine 452a. The amount of extraction solvent added in the milling subsystem 420a is measured and reported to the server 418a by a sensor 428a, and stored by the server 418a and/or in the database 414 by the server 402. Addition of extraction solvent at the extraction subsystem 420c is then based not only on a total amount of the extraction solvent that is needed for extraction, which could be stored as part of an operating program or parameters for extraction, but also on the amount of the extraction solvent that was already added at the milling subsystem 420a. In an embodiment, the amount of extraction solvent that is to be added for extraction is calculated by subtracting the amount of extraction solvent that was added at the milling subsystem 420a from the total amount of the extraction solvent that is needed for extraction.

This calculation, and control of solvent addition for extraction, can be automatic with this type of coordinated control or interaction between subsystems. In an embodiment, a controller 426c in the extraction subsystem 420c accesses or is otherwise provided with information that is stored in the database 414 and/or elsewhere to indicate how much extraction solvent was added at the milling subsystem 420a, calculates how much more extraction solvent is required for extraction, and controls one or more components such as a valve and/or a pump to dispense the calculated amount of extraction solvent from the solvent supply(ies) 470c.

Such coordination between subsystems and automated control can more accurately and reliably control usage of extraction solvent(s) relative to batch processing systems in which each processing operation is separate and distinct and/or processing systems that involve a higher degree of human for operation and/or control.

Solvent that is also or instead added at other times or locations before extraction, such as before, during, and/or after decarboxylation, are similarly taken into account in controlling addition of extraction solvent for extraction in other embodiments.

Another possible application of inter-subsystem coordinated control is in managing processing across multiple subsystems, or even end-to-end such as from milling through distillation in the example system 400 illustrated in FIGS. 4A to 4E. Some processes inevitably take longer than others in a processing system, and coordinated control can be particularly useful in optimizing or at least streamlining processing to improve such characteristics as system efficiency, equipment usage, and/or processing throughput, for example.

Any of various types of sensors implemented at any of various locations in a processing system enable collection of state information based upon which control actions are determined. Per-subsystem flow rate control and temperature control as described herein are examples of control actions. Inter-subsystem coordinated control adds a further level of control to potentially enable more effective processing management.

With per-subsystem monitoring and control, operations such as cannabis material flow rates and processing rates can be matched in an effort to avoid backup of input and/or output cannabis material within each subsystem. In some embodiments, control of one subsystem is further dependent upon conditions in one or more other subsystems.

As an example, consider a processing system in which multiple extractors are implemented in order to support continuous processing. In the event of an extractor failing or otherwise becoming inoperable, it might not be possible to maintain a current overall extraction rate. Although a lower rate of extraction might eventually be detected in an upstream subsystem such as a milling subsystem when cannabis material backs up in the processing system as a result of lower extraction throughput, in an embodiment with coordinated inter-subsystem control a change in state or condition of one subsystem is signaled to one or more other subsystems.

In this example of an extractor failure, in one embodiment a controller 426c and/or a sensor 428c in the extraction subsystem 420c in FIG. 4C detects the failure and signals the server 418c to report the failure. The server 418c determines one or more appropriate control actions and/or reports the failure to another server, such as the milling subsystem server 418a and/or the central server 402. Such reporting enables control actions to be determined by one or more server-based controllers or one or more controllers in a different subsystem, to reduce processing throughput. Processing control can thereby adapt more quickly as operating conditions change, to backpressure cannabis material processing and/or supply from upstream stations in this example.

In the above example of an extractor failure, the failure is also or instead reported to, and/or used to control one or more components of, one or more downstream subsystems in some embodiments. Reducing downstream processing throughput by executing control actions that are based on a state or condition affecting an upstream processing subsystem potentially avoids a cannabis material supply shortage at the downstream subsystem(s).

An extractor failure is described above as an illustrative example. Similar recovery procedures are also or instead provided for failure of other processing system devices or equipment in other embodiments.

Control is also or instead responsive to less dramatic changes operating conditions in other embodiments. Suppose, for example, that a buildup of milled cannabis material is detected by a controller 426a or the server 418a in the milling subsystem 420a based on weight measurements taken by a scale 430a-2. Examples of per-subsystem or intra-subsystem control actions include reducing the speed of a milling machine 452a and reducing flow rate through the transfer mechanism(s) 460a-1 and/or 460a-2 to reduce, slow, or eliminate the buildup. In another embodiment, one or more control actions for other subsystems are also or instead determined and applied. In this example of buildup of milled cannabis material, examples of such control actions include increasing a flow rate for transfer of milled cannabis material out of the vessel(s) 456a, increasing a speed of a next downstream processing subsystem such as a decarboxylation subsystem or an extraction subsystem, increasing a speed of further downstream cannabis material transfer from that next downstream processing subsystem, and so on along at least part of a processing system. Additional examples include decreasing a flow rate for transfer of cannabis material from a next upstream processing subsystem, decreasing a processing speed of the next upstream processing subsystem, decreasing a speed of input cannabis material transfer to that next upstream processing subsystem, and so on along at least another part of a processing system.

In some embodiments, such coordinated control between subsystems is applied to other processing management tasks such as determining when to bring, or not bring, additional processing capacity online. In the above example of an inoperable extractor, suppose that the extractor is replaced, repaired, or otherwise ready to resume operation. A controller 426c or a sensor 428c in the extraction system 420c detects and reports the availability of the extractor to the server 418c in an embodiment, and one or more control actions to increase supply of input cannabis material from upstream processing are determined by one or more server-based controllers and/or one or more other subsystem controllers, such as controllers 426a, 426b in milling subsystem 420a and decarboxylation subsystem 420b. The additional extraction capacity can then be brought online when additional input cannabis material is available for extraction, or held offline to avoid cannabis material shortage or under-run as a result of insufficient upstream processing capacity to supply cannabis material at a higher flow rate.

Similar control coordination is applied in other embodiments to bring processing equipment or components offline, to reduce processing capacity or throughput, and/or to determine that additional processing capacity should not be brought online. For example, in an embodiment, a controller 426a, one or more server-based controllers, and/or one or more controllers in another subsystem determines that additional milling capacity (an additional milling machine for example) should not be brought online because there is not sufficient downstream processing capacity (extraction capacity for example)to avoid downstream backup of cannabis material.

Many of the illustrative embodiments described above relate to operation of a processing system, stations, and substations. Various control embodiments are also contemplated. Control embodiments may involve one or more controllers, examples of which are provided elsewhere herein. Although the example control embodiments described below by way of example refer to controllers, it should be appreciated that a controller could be implemented, for example, using hardware, firmware, one or more components that execute software stored in one or more non-transitory memory devices, including one or more computers or other electronic devices. Control embodiments may involve not only one or more controllers, but also other components such as one or more sensors, one or more scales, one or more storage devices, one or more input devices, and/or one or more servers, for such purposes as collecting or providing measurements and/or other data that may be used in controlling an operation, processing, system, station, or substation, for example.

In one embodiment, a system includes one or more controllers to control operation of a first station to reduce size of a cannabis plant material, and to control operation of a second station that is coupled to receive a continuous supply of reduced size cannabis plant material from the first station and to obtain from the reduced size cannabis plant material a cannabis extract including at least one cannabinoid and/or terpene. A single controller or multiple different controllers may be involved.

The one or more controllers may be configured to coordinate operation of the first station and operation of the second station with the continuous supply, to streamline processing and avoid overflow and/or underflow of input and/or output cannabis material at or between the first station and the second station, for example.

The one or more controllers may include a controller to coordinate, with operation of the first station and operation of the second station, operation of a transfer mechanism to transfer the reduced size cannabis plant material from the first station to the second station. The transfer mechanism controller may be a separate controller, or a controller that controls the first station and/or the second station may also control the transfer mechanism.

The one or more controllers may include a controller, which may be a controller of the first station, a controller of the second station, a controller of the transfer mechanism, or another controller, to coordinate operation of one or more further stations with each other and/or with operation of either or both of the first station and the second station. Examples of coordinating operation of multiple components, stations, or substations are provided elsewhere herein.

The one or more controllers may also or instead include a controller, which again may be a separate controller or a controller of another component, station, or substation for example, to coordinate operation of one or more transfer mechanisms to transfer cannabis material to or from the one or more further stations with operation of the one or more further stations and/or with operation of either or both of the first station and the second station. Examples of coordinating transfer mechanisms or transfer of cannabis material with operation of components, stations, or substations are provided elsewhere herein.

In this example of a first station to reduce size of cannabis plant material and a second station to obtain a cannabis extract, the one or more further stations may include any one or more of: a decarboxylation station; a winterization station; a distillation station; a separation station; and a pre-treatment station, for example. Embodiments may also or instead include other components, stations, or substations.

In another embodiment, a system includes one or more controllers to control operation of a first station to process cannabis plant material to obtain a cannabis extract including at least one cannabinoid and/or terpene, and to control operation of a second station that is coupled to receive a continuous transfer of the cannabis extract from the first station and to purify the cannabis extract.

The one or more controllers may be configured to coordinate operation of the first station and operation of the second station with continuous transfer of the cannabis extract.

The first station in this example may include an extraction vessel to hold the cannabis extract in an extraction solvent, and the one or more controllers may include a controller to control continuous withdrawal of a portion of the extraction solvent containing the cannabis extract from the extraction vessel so as to substantially maintain at least a minimum volume of plant material and extraction solvent in the extraction vessel.

According to another embodiment, a system includes one or more controllers to: control continuous supply of cannabis plant extract to a precipitation separator that comprises a cooling path to cool the cannabis plant extract, as the cannabis plant extract passes through the cooling path at a flow rate, to induce precipitation of an undesirable component from the cannabis plant extract; and to control a rate of heat extraction from the cooling path. The cannabis plant extract includes an extraction solvent, with one or more cannabinoids and the undesirable component in solution in the extraction solvent, and the undesirable component has a precipitation temperature at which the one or more cannabinoids remain in solution in the extraction solvent. The one or more controllers are configured to control the rate of heat extraction from the cooling path in relation to the flow rate to bring the cannabis plant extract passing through the cooling path to a temperature that is below the precipitation temperature.

The precipitation separator may be or be part of a winterization station, and the one or more controllers may include a controller to control a rate of transfer of the cannabis plant extract to the precipitation separator to substantially match a rate of winterization.

The one or more controllers may include a controller to control the flow rate. The controller to control the flow rate may be configured to control the flow rate using one or more valves at one or both of an inlet of the cooling path and an outlet of the cooling path, and/or using one or more pumps, for example.

The one or more controllers may include a controller to coordinate processing of cannabis material at one or more further stations with each other and/or with processing of the cannabis plant extract at a winterization station that includes the precipitation separator.

The one or more controllers may include a controller to coordinate transfer of cannabis material to or from the one or more further stations with the processing at the one or more further stations and/or with the processing of the cannabis plant extract at the winterization station.

In this example embodiment of control in conjunction with a precipitation separator, the one or more further stations may include any one or more of: a pre- treatment station; a milling station; an extraction station; a decarboxylation station; a distillation station; and a separation station, for example.

These and other examples of processing and processing equipment control are intended solely for illustrative purposes. Other control embodiments are also possible.

Various apparatus or system embodiments are described above. Features that are described primarily with reference to stations may also or instead be applicable to substations, and similarly features that are described primarily with reference to substations may also or instead be applicable to stations. Features that are disclosed herein in the context of apparatus or systems are also or instead applicable to method embodiments. Similarly, features that are disclosed herein in the context of methods are also or instead applicable to apparatus or systems. Several method embodiments are described by way of example below.

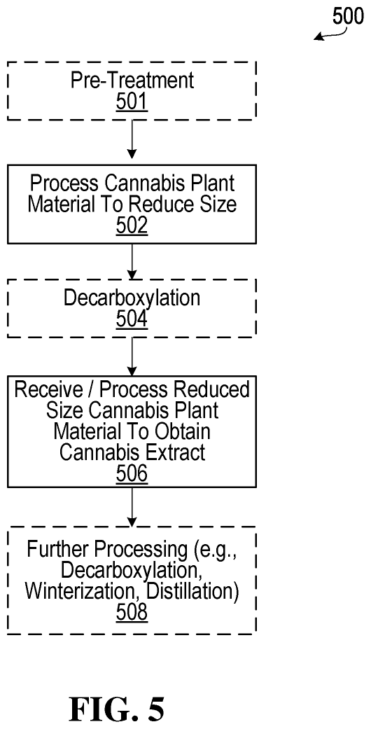

FIG. 5 is a flow diagram illustrating a method 500 according to another embodiment. Some embodiments involve pre-treatment of cannabis plant material at pre-treatment station as shown at 501, and examples of pre-treatment are provided elsewhere herein. As shown at 502, the example method 500 involves processing a cannabis plant material, at a first station for example, to reduce size of the cannabis plant material and produce reduced size cannabis plant material. The processing at 502 involves processing pre-treated cannabis plant material from a pre-treatment station in some embodiments. Decarboxylation at 504 is optional, and therefore is shown in FIG. 5 in a dashed line box. At 506, the reduced size cannabis plant material is received and processed, at a second station that is coupled to receive the reduced size cannabis plant material from the first station in some embodiments, to obtain from the reduced size cannabis plant material a cannabis extract including at least one cannabinoid. The second station may be coupled to receive a continuous supply of reduced size cannabis plant material, for example.

In one embodiment that includes a first station and a second station, the second station is in fluid communication with the first station, and is in this manner coupled to receive the reduced size cannabis plant material from the first station. Integration of the first and second stations together in a single device or piece of processing equipment is another option to couple the second station to receive the reduced size cannabis plant material from the first station. Integration examples are disclosed elsewhere herein.

Processing stations are coupled together through a transfer mechanism in some embodiments. In such an embodiment with a first station and a second station for example, a method also includes controlling the transfer mechanism to transfer the reduced size cannabis plant material from the first station to the second station.

An example of processing reduced size cannabis plant material at a second station at 506 is extracting the reduced size cannabis plant material with an extraction solvent. Solvent-based extraction involves contacting the reduced size cannabis plant material with the extraction solvent. Another example of processing the reduced size cannabis plant material at the second station at 506 is performing mechanical extraction on the reduced size cannabis plant material.

An extraction solvent need not necessarily only be used for extraction. For example, in some embodiments processing cannabis plant material at a first station at 502 involves contacting the cannabis plant material with the extraction solvent to transfer the reduced size cannabis plant material from the first station to the second station. In some embodiments, extraction involves a warm solvent extraction process that further causes decarboxylation of the at least one cannabinoid.

A processing method involves additional processing in some embodiments, as shown by way of example in FIG. 5 at 508.

In some embodiments, a method also involves processing the cannabis extract, at a winterization station that is coupled to receive the cannabis extract from the second station, to winterize the cannabis extract. The winterization station is in fluid communication with the second station in some embodiments. The winterization station may be coupled to receive a continuous supply of the cannabis extract, for example.

Transfer of cannabis extract through a transfer mechanism, as disclosed elsewhere herein, involves controlling the transfer mechanism to transfer the cannabis extract, in a continuous supply for example, from the second station to the winterization station. In some embodiments that implement solvent-based extraction with winterization, a method involves transferring the cannabis extract from the second station to a winterization station using the extraction solvent, and processing the cannabis extract, at the winterization station, to winterize the cannabis extract.

Processing cannabis extract at a winterization station involves contacting the cannabis extract with a winterization solvent in some embodiments.

Another example of optional further processing at 508 is distillation. In an embodiment, a method involves processing winterized cannabis extract, at a distillation station that is coupled to receive the winterized cannabis extract from the winterization station, to purify the at least one cannabinoid. The distillation station is in fluid communication with the winterization station in some embodiments. The distillation station may be coupled to receive a continuous supply of the winterized cannabis extract from the winterization station, for example.

Some methods involve controlling a transfer mechanism to transfer the winterized cannabis extract from the winterization station to the distillation station.

When a winterization solvent is used in winterization of cannabis extract, a method could involve transferring the winterized cannabis extract, in a continuous supply for example, from the winterization station to a distillation station using the winterization solvent and then processing the winterized cannabis extract at the distillation station to purify the at least one cannabinoid.

Distillation need not be implemented in combination with winterization. In some embodiments, a method involves processing the cannabis extract, at a distillation station that is coupled to receive the cannabis extract from the second station, to purify the at least one cannabinoid. The distillation station may be coupled to receive a continuous supply of the cannabis extract from the second station, for example. As in other embodiments noted above, the distillation station could be in fluid communication with the second station, and/or a method could involve controlling a transfer mechanism to transfer the cannabis extract, in a continuous supply for example, from the second station to the distillation station.

Isolation and/or separation, generally referred to herein as “separation”, are provided in some embodiments, to purify one or more cannabinoids in a crude extract and/or to further purify one or more cannabinoids in a distillate for example. In one such embodiment, a method involves processing cannabis extract, at a separation station that is coupled to receive the cannabis extract from another station, in a continuous supply for example, to separate at least one cannabinoid and/or terpene from the cannabis extract. Another embodiment involves processing winterized cannabis extract, at a separation station that is coupled to receive the winterized cannabis extract from a winterization station, in a continuous supply for example, to separate at least one cannabinoid and/or terpene from the winterized cannabis extract. In a further embodiment, both distillation and separation are provided, and a method involves processing a distillate, at a separation station that is coupled to receive the distillate from a distillation station, in a continuous supply for example, to further purify at least one cannabinoid and/or terpene.

An extraction solvent used in extraction at the second station is also used in transferring the cannabis extract from the second station to a distillation station in some embodiments. The cannabis extract, transferred to the distillation station in the extraction solvent, is then processed at the distillation station to purify the at least one cannabinoid.

Some embodiments relate to integrated processing stations, and integrated processing is applied in some method embodiments as well. Consider, for example, a method that involves processing a cannabis plant material at a first station to obtain a cannabis extract including at least one cannabinoid and/or terpene, and processing the cannabis extract, at a second station that is coupled to receive the cannabis extract from the first station, to purify the cannabis extract. The second station may be coupled to receive the cannabis extract that is continuously transferred from the first station, for example. A method may include continuously transferring at least a portion of the cannabis extract from the first station to the second station. The processing at the first station may involve processing the cannabis plant material with an extraction solvent, and continuously transferring may involve transferring at least the portion of the cannabis extract to the second station in at least a portion of the extraction solvent. In another embodiment, the processing at the first station involves performing mechanical extraction on the cannabis plant material.

In an embodiment, the processing at the first station involves integrated processing, including processing the cannabis plant material at a first substation of the first station to reduce size of the cannabis plant material (at 502 in FIG. 5 for example), and processing reduced size cannabis plant material from the first substation, at a second substation of the first station that is coupled to receive the reduced size cannabis plant material from the first substation, to obtain the cannabis extract from the reduced size cannabis plant material (at 506 in FIG. 5 for example).

In this example, the processing at the second station involves winterizing the cannabis extract to obtain a winterized extract in some embodiments, and possibly distilling the winterized extract to obtain the at least one cannabinoid.

In other embodiments, the processing at the second station involves distilling the cannabis extract to obtain the at least one cannabinoid.

Integrated processing also involves pre-treatment in some embodiments. For example, in an embodiment processing at the first station involves pre-treating cannabis plant material at a pre-treatment substation, and processing pre-treated cannabis plant material from the pre-treatment substation, at an extraction substation of the first station that is coupled to receive the pre-treated cannabis plant material from the pre-treatment substation, to obtain the cannabis extract from the pre-treated cannabis plant material. According to another embodiment, processing at the first station involves: pre-treating cannabis plant material at a pre-treatment substation; processing pre-treated cannabis plant material at a first substation that is coupled to receive the pre-treated cannabis plant material from the pre-treatment substation, to reduce size of the pre-treated cannabis plant material; and processing reduced size cannabis plant material from the first substation, at a second substation of the first station that is coupled to receive the reduced size cannabis plant material from the first substation, to obtain the cannabis extract from the reduced size cannabis plant material.

Distillation is described above as an example of second station processing. In other embodiments, processing at the second station involves performing separation to separate at least one cannabinoid and/or terpene in the cannabis extract and obtain the at least one cannabinoid and/or terpene. Another example of processing at the second station involves performing separation to separate at least one cannabinoid and/or terpene in a winterized extract and obtain the at least one cannabinoid and/or terpene. In further embodiments, processing at the second station involves performing separation to further purify at least one cannabinoid and/or terpene after distillation, by separating the at least one cannabinoid and/or terpene in a distillate that includes the at least one cannabinoid and/or terpene.

As in other embodiments, the first station may include an extraction vessel to hold the cannabis extract in an extraction solvent. A method may involve continuously withdrawing a portion of the extraction solvent containing the cannabis extract from the extraction vessel so as to substantially maintain at least a minimum volume of plant material and extraction solvent in the extraction vessel. Continuously withdrawing may involve continuously withdrawing the portion of the extraction solvent containing the cannabis extract from the extraction vessel so as to substantially maintain a constant volume of plant material and extraction solvent in the extraction vessel.

The second station may include a winterization substation in fluid communication with the extraction vessel, and a method may involve transferring the extract from the extraction vessel to the winterization substation. The withdrawn portion of the extraction solvent may transfer the extract from the extraction vessel to the winterization substation.

As in other embodiments, a method may involve incorporating a winterization solvent such that the extract is in contact with the winterization solvent in the winterization substation, and/or involve winterizing the extract.

The second station may include a distillation substation in fluid communication with the winterization substation. In some embodiments, a method may involve transferring winterized extract from the winterization substation to the distillation substation, and may also or instead involve distillation of the winterized extract to purify the at least one cannabinoid and/or terpene.

In an embodiment, the second station includes a distillation substation in fluid communication with the extraction vessel. The withdrawn portion of the extraction solvent may transfer the extract from the extraction vessel to the distillation substation. In some embodiments, a method involves distillation of the extract to purify the at least one cannabinoid and/or terpene.

A method may involve separation of the at least one cannabinoid and/or terpene in the cannabis plant extract withdrawn from the extraction vessel to obtain the at least one cannabinoid and/or terpene, separation of the at least one cannabinoid and/or terpene in winterized extract from the winterization substation, and/or separation of a distillate comprising the at least one cannabinoid and/or terpene, to further purify the at least one cannabinoid and/or terpene.

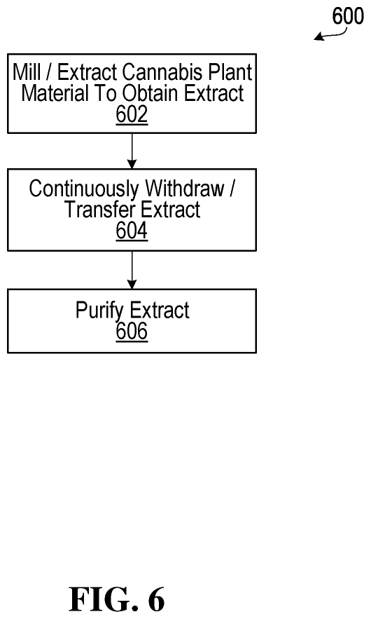

FIG. 6 is a flow diagram illustrating a method according to a further embodiment. The example method 600 in FIG. 6 involves processing a cannabis plant material, at an extraction station for example, to obtain a cannabis extract including at least one cannabinoid. This is shown by way of example at 602, which also illustrates that other operations such as milling are performed in some embodiments.

Some embodiments also involve continuously transferring at least a portion of the cannabis extract to a purification station that is coupled to receive the cannabis extract from the extraction station. This is shown at 604.

The processing at an extraction station involves processing the cannabis plant material with an extraction solvent in some embodiments, in which case the transferring could involve transferring at least the portion of the cannabis extract in at least a portion of the extraction solvent. Processing at an extraction station also or instead includes performing mechanical extraction on the cannabis plant material in some embodiments.

Examples of a purification station include a winterization station and a distillation station. In the case of a winterization station, purifying an extract as shown at 606 involves winterizing the cannabis extract in presence of a winterization solvent to obtain a winterized extract in some embodiments. If a purification station also includes comprises a distillation station, then a method also includes distillation of the winterized extract to obtain the at least one cannabinoid in some embodiments.

A purification station need not necessarily include both a winterization station and a distillation station. A method involving purification at a distillation station without a winterization station includes, in an embodiment, distillation of the cannabis extract to obtain the at least one cannabinoid.

In some embodiments, a purification station also or instead includes a separation station, to purify one or more cannabinoids in a crude extract and/or to further purify one or more cannabinoids in a distillate for example. In an embodiment, the purification station includes a separation station and a method involves separation of the at least one cannabinoid and/or terpene in the cannabis extract to obtain the at least one cannabinoid and/or terpene from the cannabis extract. In another embodiment, the purification station includes a winterization station and a separation station, and a method involves separation of the at least one cannabinoid and/or terpene in the winterized cannabis extract to obtain the at least one cannabinoid and/or terpene from the winterized cannabis extract. According to a still further embodiment, the purification station includes a distillation station and a separation station, and a method involves separation of the at least one cannabinoid and/or terpene in a distillate, to further purify the at least one cannabinoid and/or terpene.

As noted herein, features disclosed in the context of method embodiments are also applicable to system or apparatus embodiments. Considering the foregoing description of a method with reference to FIG. 6, a system embodiment includes an extraction station to obtain from a cannabis plant material a cannabis extract including at least one cannabinoid, a purification station to purify the cannabis extract, and a transfer mechanism, coupled to the extraction station and to the purification station, to continuously transfer at least a portion of the cannabis extract from the extraction station to the purification station.

In an embodiment, the extraction station is configured to obtain the cannabis extract by processing the cannabis plant material with an extraction solvent, and the transfer mechanism is configured to transfer at least the portion of the cannabis extract to the purification station in at least a portion of the extraction solvent.

The purification station in some embodiments includes a winterization station to winterize the cannabis extract in presence of a winterization solvent to obtain a winterized extract.

The purification station includes, in other embodiments, a distillation station, coupled to receive the cannabis extract from the extraction station, to distill the cannabis extract to obtain the at least one cannabinoid; or a distillation station, coupled to receive the winterized extract from the winterization station, to distill the winterized extract to obtain the at least one cannabinoid.

A purification station includes a separation station in some embodiments, instead of or in addition to a winterization station and/or a distillation station.

The example method 600 is also illustrative of other embodiments, including a process that involves providing an extraction vessel containing a cannabis plant extract in an extraction solvent and incorporating a cannabis plant material and a volume of extraction solvent into the vessel. These operations are examples of operations performed in extraction at 602 in some embodiments.

Another example of an operation that is performed at 604 in some embodiments is continuously withdrawing a portion of the extraction solvent containing the cannabis plant extract from the vessel, so as to substantially maintain a constant volume of plant material and extraction solvent in the vessel in some embodiments. The cannabis plant extract includes at least one cannabinoid and/or terpene.

Continuously withdrawing a portion of the extraction solvent containing the cannabis plant extract from the extraction vessel at 604 may involve continuously withdrawing so as to substantially maintain at least a minimum volume of plant material and extraction solvent in the extraction vessel. A constant volume is substantially maintained in some embodiments, and in other embodiments continuous withdrawal at 604 is to substantially maintain at least a minimum volume, but not necessarily a constant volume, of the plant material and extraction solvent in the extraction vessel.

The minimum volume may be selected or determined to avoid underflow of the extraction vessel and/or insufficient extraction solvent in the extraction vessel, for example. A maximum volume may also or instead be selected or determined, to avoid a bottleneck or overflow of the extraction vessel, for example. Continuously withdrawing a portion of the extraction solvent containing the cannabis plant extract from the vessel may be such that at most the maximum volume is substantially maintained. In another embodiment, the continuous withdrawing is so as to substantially maintain a volume in the extraction vessel between the minimum volume and the maximum volume. The volume that is substantially maintained in the extraction vessel may or may not be constant.

The vessel is in fluid communication with a winterization station in some embodiments.

A method could involve transferring the extract from the vessel to the winterization station. In some embodiments, the withdrawn portion of the extraction solvent transfers the extract from the vessel to the winterization station.

For winterization, some method embodiments involve incorporating a winterization solvent such that the extract is in contact with the winterization solvent in the winterization station.

Winterizing the extract to obtain a winterized extract is one example of purification at 606.

Distillation could be provided in addition to winterization, and in some embodiments the winterization station is in fluid communication with a distillation station. A method implementing distillation with winterization include, in some embodiments, transferring the winterized extract to the distillation station, and distillation of the extract to purify the at least one cannabinoid and/or terpene.

For distillation without winterization, in some embodiments the vessel from which a portion of the extraction solvent containing the cannabis plant extract is withdrawn is in fluid communication with a distillation station. The withdrawn portion of the extraction solvent transfers the extract from the vessel to the distillation station in some embodiments. Distillation of the extract to purify the at least one cannabinoid and/or terpene is another example of purification at 606.

A process also includes separation in some embodiments, such as: separation of the at least one cannabinoid and/or terpene in the cannabis plant extract to obtain the at least one cannabinoid and/or terpene; separation of the at least one cannabinoid and/or terpene in the winterized extract to purify the at least one cannabinoid and/or terpene; and/or separation of a distillate comprising the at least one cannabinoid and/or terpene, to further purify the at least one cannabinoid and/or terpene.

A system embodiment to implement such methods or processes includes an extraction vessel containing a cannabis plant extract in an extraction solvent, and a transfer mechanism coupled to the extraction vessel and configured to continuously withdraw a portion of the extraction solvent containing the cannabis plant extract from the vessel so as to substantially maintain a constant volume of plant material and extraction solvent in the vessel. As noted elsewhere herein, a constant volume may or may not be substantially maintained. The transfer mechanism may be configured to continuously withdraw a portion of the extraction solvent containing the cannabis plant extract from the vessel so as to substantially maintain at least a minimum volume, at most a maximum volume, or a volume between a minimum volume and a maximum volume in the vessel.

A winterization station is coupled to the transfer mechanism in some embodiments, to receive the withdrawn portion of the extraction solvent containing the cannabis plant extract. The winterization station is configured to contact the extract with a winterization solvent in some embodiments.

Some system embodiments include a distillation station in fluid communication with the winterization station, and possibly a transfer mechanism, coupled to the winterization station and to the distillation station, to transfer the winterized extract to the distillation station.

In other embodiments a distillation station is coupled to the transfer mechanism that is coupled to the extraction vessel, to receive the withdrawn portion of the extraction solvent containing the cannabis plant extract.

A separation station is coupled to the transfer mechanism, to receive the withdrawn portion of the extraction solvent containing the cannabis plant extract in some embodiments.

In some embodiments that include a winterization station, a separation station in fluid communication with the winterization station. A transfer mechanism may be coupled to the winterization station and to the separation station, to transfer winterized extract to the separation station.

In some embodiments that include a distillation station, a separation station in fluid communication with the distillation station. A transfer mechanism may be coupled to the separation station and to the distillation station, to transfer a distillate from the distillation station to the separation station.

Other embodiments are also possible. Winterization at 508 in FIG. 5 and purification at 606 in FIG. 6 represent examples of a process for removing an undesirable component from a cannabis plant extract. A cannabis plant extract that is produced by solvent extraction includes an extraction solvent, with one or more cannabinoids and an undesirable component in solution in the extraction solvent. Winterization exploits a property of the undesirable component having a precipitation temperature at which the one or more cannabinoids remain in solution in the extraction solvent.

In some embodiments, a removal process or method involves continuously supplying cannabis plant extract to a precipitation separator that includes a cooling path to cool the cannabis plant extract, as the cannabis plant extract is passing through the cooling path at a flow rate, to induce precipitation of the undesirable component. A rate of heat extraction from the cooling path in relation to the flow rate is controlled to bring the cannabis plant extract passing through the cooling path to a temperature that is below the precipitation temperature to induce the precipitation of the undesirable component, and the precipitated undesirable component is removed from cooled cannabis plant extract. Examples of such a method or process, and system or apparatus embodiments to carry out such a method or process, are disclosed elsewhere herein and are also referenced by way of example below.

The precipitation separator may be or be part of a winterization station. A process may involve controlling a rate of transfer of the cannabis plant extract to the precipitation separator to substantially match a rate of winterization.

A process may involve controlling the flow rate through the cooling path. Options for controlling the flow rate include controlling the flow rate using one or more valves at one or both of an inlet of the cooling path and an outlet of the cooling path, involve controlling the flow rate using one or more pumps, and, if the cannabis plant extract is gravity fed through the cooling path for example, adjusting any one or more of: an angle of the cooling path with respect to vertical, shape of the cooling path, size of the cooling path, and drag exerted on the cannabis plant extract by the cooling path, to control the flow rate. Adjusting the drag exerted on the cannabis plant extract by the cooling path by changing a width or a cross-sectional area of the cooling path, for example.

Removing the undesirable component may involve repeatedly or continuously removing the undesirable component from the cooled cannabis extract as it flows through the cooling path, for example by filtering, using one or more filters, using one or more membranes, or a brush or filter periodically or continuously sweeping to catch or trap the undesirable component.

A process may involve depositing the undesirable component in a container.

In an embodiment, removal of an undesirable component is implemented or enabled by a system that includes a precipitation separator to receive a continuous supply of cannabis plant extract. The precipitation separator includes a cooling path to cool the cannabis plant extract, as the cannabis plant extract passes through the cooling path at a flow rate, to induce precipitation of the undesirable component. A system may also include a controller to control a rate of heat extraction from the cooling path in relation to the flow rate to bring the cannabis plant extract passing through the cooling path to a temperature that is below the precipitation temperature.

The precipitation separator may be or be part of a winterization station, as noted at least above.

The controller, or a further controller, may be configured to control a rate of transfer of the cannabis plant extract to the precipitation separator to substantially match a rate of winterization. Flow rate and rate of winterization may be sensed by one or more sensors, input by a user, and/or otherwise available or accessible for flow control.

The controller or a further controller may also or instead be configured to control the flow rate through the cooling path, for example by using valves at one or both of an inlet of the cooling path and an outlet of the cooling path, and/or using one or more pumps. In an embodiment in which the cannabis plant extract is gravity fed through the cooling path, for example, the controller or a further controller may be configured to control the flow rate by adjusting any one or more of: angle of the cooling path with respect to vertical, shape of the cooling path, size of the cooling path, and drag exerted on the cannabis plant extract by the cooling path, to control the flow rate. As noted at least above, adjusting the drag exerted on the cannabis plant extract by the cooling path may involve changing a width or a cross-sectional area of the cooling path.

In some embodiments, heat is extracted from the cooling path by a heat exchanger, and accordingly a system may include a heat exchanger to extract heat from the cooling path.

A system may include an element for removal of precipitated undesirable component from cooled cannabis plant extract as it flows through the cooling path. Examples of such an element include: one or more filters, one or more membranes, one or more centrifuges, and a brush. Multiple different types of elements may be used for removal of precipitated undesirable component.

A system may include a container, and a pipe to enable the undesirable component to be removed and to deposit the undesirable component in the container, as described by way of example at least above.

In an embodiment, a system includes an output coupled to an input of a heating element to allow winterized cannabis plant extract to enter the heating element. Such a system may include a filter and/or another element or device to prevent the undesirable component from flowing into the heating element. Winterized cannabis plant extract flows to the heating element in a continuous stream in some embodiments.

The removal process outlined above is illustrative of another example method, and other embodiments including control methods are also possible. For example, one such control method involves controlling processing of a cannabis plant material at a first station to reduce size of the cannabis plant material and produce reduced size cannabis plant material; and controlling processing of the reduced size cannabis plant material at a second station that is coupled to receive a continuous supply of the reduced size cannabis plant material from the first station and to obtain from the reduced size cannabis plant material a cannabis extract including at least one cannabinoid and/or terpene.

Controlling processing at the first station and controlling processing at the second station may involve coordinating the processing at the first station and the processing at the second station with the continuous supply.

Such a method may involve controlling transfer of the reduced size cannabis plant material from the first station to the second station.

In some embodiments, a method involves coordinating processing at one or more further stations with each other and/or with the processing at either or both of the first station and the second station.

Some embodiments may involve coordinating transfer of cannabis material to or from the one or more further stations with the processing at the one or more further stations and/or with the processing at either or both of the first station and the second station. As noted above, the one or more further stations may include any one or more of: a decarboxylation station; a winterization station; a distillation station; a separation station; and a pre-treatment station, for example.

Another control method involves: controlling operation of a first station to process cannabis plant material to obtain a cannabis extract including at least one cannabinoid and/or terpene; and controlling operation of a second station that is coupled to receive the cannabis extract continuously transferred from the first station and to purify the cannabis extract.

Controlling operation of the first station and controlling operation of the second station may involve coordinating operation of the first station and operation of the second station with continuous transfer of the cannabis extract.

The first station may include an extraction vessel to hold the cannabis extract in an extraction solvent, and such a method may involve controlling continuous withdrawal of a portion of the extraction solvent containing the cannabis extract from the extraction vessel so as to substantially maintain at least a minimum volume of plant material and extraction solvent in the extraction vessel.

In an embodiment, a further control method involves: controlling continuous supply of cannabis plant extract to a precipitation separator that comprises a cooling path to cool the cannabis plant extract, as the cannabis plant extract passes through the cooling path at a flow rate, to induce precipitation of an undesirable component from the cannabis plant extract, the cannabis plant extract including an extraction solvent, with one or more cannabinoids and the undesirable component in solution in the extraction solvent, the undesirable component having a precipitation temperature at which the one or more cannabinoids remain in solution in the extraction solvent; and controlling a rate of heat extraction from the cooling path in relation to the flow rate to bring the cannabis plant extract passing through the cooling path to a temperature that is below the precipitation temperature.

The precipitation separator may be or be part of a winterization station, and a method may involve controlling a rate of transfer of the cannabis plant extract to the precipitation separator to substantially match a rate of winterization.

A method may involve controlling the flow rate.

Controlling the flow rate may involve controlling the flow rate using one or more valves at one or both of an inlet of the cooling path and an outlet of the cooling path, and/or using one or more pumps.

A method may involve coordinating processing of cannabis material at one or more further stations with each other and/or with processing of the cannabis plant extract at a winterization station that includes the precipitation separator.

In an embodiment, a method involves coordinating transfer of cannabis material to or from the one or more further stations with the processing at the one or more further stations and/or with the processing of the cannabis plant extract at the winterization station.

The one or more further stations may include, for example, any one or more of: a pre-treatment station; a milling station; an extraction station; a decarboxylation station; a distillation station; and a separation station.

Although the foregoing has been described with reference to certain specific embodiments, various modifications thereof will be apparent to those skilled in the art without departing from the scope of the invention as defined by the claims.

For example, embodiments disclosed in the context of a system or apparatus or in the context of a method or process not exclusive to system/apparatus or method/process applications. Features of system/apparatus embodiments are potentially applicable to method/process embodiments, and vice versa.

Features are also not intended to be restricted to implementation in any particular combinations. A feature that is disclosed herein in the context of an embodiment that also includes other features are combinable with different disclosed features, and are not in any way limited to combinations that are explicitly disclosed.

It should also be noted that the present disclosure concentrates primarily on such aspects as control, monitoring, and operation of cannabis material processing and processing systems. Automated and/or integrated features may have other applications, instead of or in addition to those disclosed herein. For example, automated monitoring and/or control may enable production of reports on processing capacity and/or any of various other processing parameters, for such purposes as financial reporting and/or reporting to regulators. Processors of cannabis material and producers of cannabis products may have to report on inventory, for instance, and this has historically been a manual process. With automated processing/production, data on inventory can be live and potentially more accurate than with manual processes. These features could provide significant savings in terms of human resource cost and administration time. Thus, another potential advantage of using an automated process is report generation.

Any module, component, or device exemplified herein that executes instructions may include or otherwise have access to a non-transitory computer/processor readable storage medium or media for storage of information, such as computer/processor readable instructions, data structures, program modules, and/or other data. A non-exhaustive list of examples of non-transitory computer/processor readable storage media includes magnetic cassettes, magnetic tape, magnetic disk storage or other magnetic storage devices, optical disks such as compact disc read-only memory (CD-ROM), digital video discs or digital versatile disc (DVDs), Blu-ray Disc™, or other optical storage, volatile and non-volatile, removable and non-removable media implemented in any method or technology, random-access memory (RAM), read-only memory (ROM), electrically erasable programmable read-only memory (EEPROM), flash memory or other memory technology. Any such non-transitory computer/processor storage media may be part of a device or accessible or connectable thereto. Any application or module herein described may be implemented using computer/processor readable/executable instructions that may be stored or otherwise held by such non-transitory computer/processor readable storage media.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims.

1. A process for removing an undesirable component from a cannabis plant extract, the cannabis plant extract including an extraction solvent, with one or more cannabinoids and the undesirable component in solution in the extraction solvent, the undesirable component having a precipitation temperature at which the one or more cannabinoids remain in solution in the extraction solvent, the process comprising:
continuously supplying cannabis plant extract to a precipitation separator that comprises a cooling path to cool the cannabis plant extract, as the cannabis plant extract passes through the cooling path at a flow rate, to induce precipitation of the undesirable component;
controlling a rate of heat extraction from the cooling path in relation to the flow rate to bring the cannabis plant extract passing through the cooling path to a temperature that is below the precipitation temperature;
removing precipitated undesirable component from cooled cannabis plant extract.

2. The process of claim 1, wherein the precipitation separator comprises a winterization station, the process further comprising either or both of:
controlling a rate of transfer of the cannabis plant extract to the precipitation separator to substantially match a rate of winterization:
controlling the flow rate.

3. (canceled)

4. The process of claim 2, wherein controlling the flow rate comprises controlling the flow rate using either or both of:
one or more valves at one or both of an inlet of the cooling path and an outlet of the cooling path;
one or more pumps.

5. (canceled)

6. The process of claim 1, wherein the cannabis plant extract is gravity fed through the cooling path, the process further comprising:
adjusting any one or more of: an angle of the cooling path with respect to vertical, shape of the cooling path, size of the cooling path, and drag exerted on the cannabis plant extract by the cooling path, to control the flow rate.

7. (canceled)

8. The process of claim 6, wherein the adjusting comprises adjusting the drag exerted on the cannabis plant extract by the cooling path by changing a width or a cross-sectional area of the cooling path.

9. (canceled)

10. The process of claim 1, wherein the removing comprises any one or more of:
repeatedly or continuously removing the undesirable component from the cooled cannabis extract as it flows through the cooling path;
filtering;
using one or more filters;
using one or more membranes.

11-13. (canceled)

14. The process of claim 1, wherein the removing comprises a brush or filter periodically or continuously sweeping to catch or trap the undesirable component.

15. (canceled)

16. A system for removing an undesirable component from a cannabis plant extract, the cannabis plant extract including an extraction solvent, with one or more cannabinoids and the undesirable component in solution in the extraction solvent, the undesirable component having a precipitation temperature at which the one or more cannabinoids remain in solution in the extraction solvent, the system comprising:
a precipitation separator to receive a continuous supply of cannabis plant extract, the precipitation separator comprising a cooling path to cool the cannabis plant extract, as the cannabis plant extract passes through the cooling path at a flow rate, to induce precipitation of the undesirable component;
a controller to control a rate of heat extraction from the cooling path in relation to the flow rate to bring the cannabis plant extract passing through the cooling path to a temperature that is below the precipitation temperature.

17. The system of claim 16, wherein the precipitation separator comprises a winterization station, wherein the controller or a further controller is configured to control either or both of:
a rate of transfer of the cannabis plant extract to the precipitation separator to substantially match a rate of winterization;
the flow rate.

18. (canceled)

19. The system of claim 17, wherein the controller or the further controller is configured to control the flow rate using either or both of:
valves at one or both of an inlet of the cooling path and an outlet of the cooling path;
one or more pumps.

20-21. (canceled)

22. The system of claim 16, wherein the cannabis plant extract is gravity fed through the cooling path, wherein the controller or a further controller is configured to control the flow rate by adjusting any one or more of: angle of the cooling path with respect to vertical, shape of the cooling path, size of the cooling path, and drag exerted on the cannabis plant extract by the cooling path, to control the flow rate.

23. The system of claim 22, wherein the wherein the controller or the further controller is configured to control the flow rate by adjusting the drag exerted on the cannabis plant extract by the cooling path by changing a width or a cross-sectional area of the cooling path.

24. (canceled)

25. The system of claim 16, comprising an element for removal of precipitated undesirable component from cooled cannabis plant extract as it flows through the cooling path, the element comprising any one or more of:
one or more filters;
one or more membranes;
one or more centrifuges;
a brush.

26-33. (canceled)

34. A method comprising:
controlling continuous supply of cannabis plant extract to a precipitation separator that comprises a cooling path to cool the cannabis plant extract, as the cannabis plant extract passes through the cooling path at a flow rate, to induce precipitation of an undesirable component from the cannabis plant extract, the cannabis plant extract including an extraction solvent, with one or more cannabinoids and the undesirable component in solution in the extraction solvent, the undesirable component having a precipitation temperature at which the one or more cannabinoids remain in solution in the extraction solvent;
controlling a rate of heat extraction from the cooling path in relation to the flow rate to bring the cannabis plant extract passing through the cooling path to a temperature that is below the precipitation temperature.

35. The method of claim 34, wherein the precipitation separator comprises a winterization station, the method further comprising either or both of:
controlling a rate of transfer of the cannabis plant extract to the precipitation separator to substantially match a rate of winterization;
controlling the flow rate.

36. (canceled)

37. The method of claim 35, wherein controlling the flow rate comprises controlling the flow rate using one or more valves at one or both of an inlet of the cooling path and an outlet of the cooling path, or using one or more pumps.

38. The method of claim 34, wherein the precipitation separator comprises a winterization station, the method further comprising:
coordinating processing of cannabis material at one or more further stations with each other and/or with processing of the cannabis plant extract at the winterization station.

39. The method of claim 38, further comprising:
coordinating transfer of cannabis material to or from the one or more further stations with the processing at the one or more further stations and/or with the processing of the cannabis plant extract at the winterization station.

40. The method of claim 39, wherein the one or more further stations comprise any one or more of:
a pre-treatment station;
a milling station;
an extraction station;
a decarboxylation station;
a distillation station;
a separation station.

41-47. (canceled)
